(19) United States
(12) Patent Application Publication
(10) Pub. No.: US 2004/0009468 A1
(43) Pub. Date: Jan. 15, 2004

(54) Allelic variants of HERV-K18, method for analysis thereof and use in the determination of genetic predisposition for disorders involving the HERV-K18 provirus

(76) Inventors: Bernard Mach, Chambesy (CH); Bernard Conrad, Geneva (CH)

Correspondence Address: MINTZ, LEVIN, COHN, FERRIS,, GLOVSKY and POPEO, P.C., One Financial Center, Boston, MA, 02111, US

(21) Appl. No.: 10/233,958

(22) Filed: Sep. 3, 2002

Related U.S. Application Data
(60) Provisional application No. 60/316,513, filed on Aug. 31, 2001.
(60) Provisional application No. 60/316,522, filed on Aug. 31, 2001.

(51) Int. Cl. C12Q 1/70; C07H 21/04; C12N 15/86; C12N 5/06; C07K 14/005; C12N 7/00

(52) U.S. Cl. 435/5; 435/69.3; 435/320.1; 435/325; 530/350; 536/23.72; 435/235.1

(57) ABSTRACT

The invention relates to polymorphic forms of the endogenous human retrovirus HERV-K18 and to methods for determining the genotype of an individual at the HERV-K18 locus. The invention also relates to the use of the HERV-K18 genotype in the identification of predisposition of individuals to disorders involving the HERV-K 18 retrovirus, for example insulin-dependent diabetes mellitus (IDDM). The invention further relates to the combination of HERV-K18 genotyping with genotyping of additional genetic loci which are also linked to IDDM, thus providing a more effective detection method.

### RELATED APPLICATIONS

This application claims the benefit of priority under 35 U.S.C. 119(e) to copending U.S. Provisional Application No. 60/316,513, filed on Aug. 31, 2001, and No. 60/316,522, filed on Aug. 31, 2001; the entire contents of which are incorporated herein by reference. This application is also related to U.S. application Ser. No. 09/490,700, filed Jan. 24, 2000, the entire contents of which are incorporated herein by reference.

### FIELD OF THE INVENTION

The present invention relates to polymorphic forms of the endogenous human retrovirus HERV-K18, and to methods for determining the genotype of an individual at this locus. The invention also relates to the use of the HERV-K18 genotype in the identification of predisposition of individuals to disorders involving the HERV-K 18 retrovirus, for example insulin-dependent diabetes mellitus (IDDM). The invention further relates to the combination of HERV-K18 genotyping with genotyping of additional genetic loci which are also linked to IDDM, thus providing a more effective detection method.

### BACKGROUND OF THE INVENTION

Human endogenous retroviruses (HERVs) entered the human genome after fortuitous germ line integration of exogenous, retroviruses and were subsequently fixed in the general population. They may have been preserved to ensure genome plasticity and this can provide the host with new functions, such as protection from exogenous viruses and fusiogenic activity. The recently identified HERV IDDMK_{1.2}22, whose env gene has been identified as a candidate associated with aberrant activation of a subset of T cells found in the pancreas of individuals that succumbed to acute insulinitis (Conrad, 1997; Conrad, 1994), is a member of the HERV-K family. To date ten proviruses with distinct integration sites have been assigned to this family (Barbulescu et al., 1999). The provirus encoding IDDMK_{1.2}22 has not yet been characterized. However, proviruses similar to IDDMK_{1.2}22 have been described (Tönjes et al., 1999; Hasuike et al., 1999; Barbulescu et al., 1999). A sequence similar to both HERV-K18 and IDDMK_{1.2}22 has been preliminarily mapped to the CD48 gene on chromosome 1 using DNA from a single individual (Hasuike et al., 1999).

However, it has to date not been established whether a link between these homologous sequences exists, and if so, the significance of such a link.

### SUMMARY OF THE INVENTION

In the context of the present invention, IDDMK_{1.2}22 has been unambiguously assigned to the HERV-K18 locus, and it has been established that the defective HERV-K18 provirus on chromosome 1 has at least three alleles, one of which corresponds to IDDMK_{1.2}22. The integration site of the HERV-K18 provirus in the large first CD48 intron has been found to be preserved in all individuals tested. The provirus is inserted in the opposite transcriptional direction to CD48 (FIG. 1A). Allelic polymorphism has been demonstrated in the envelope gene and in the 5′ and 3′ LTRs.

The population frequency of the three HERV-K18 alleles of the envelope gene has been analyzed. The IDDMK_{1.2}22 ENV coding sequence was found in 46.6% of chromosomes and was designated allele K18.1 (FIG. 1B). Two envelope sequences similar to IDDMK_{1.2}22, but without its premature stop codon were obtained at frequencies of 42.5% (allele K18.2) and 10.80 (allele K18.3). K18.2 is identical to a published sequence (Tönjes et al., 1999). K18.3 has never previously been reported. Two additional variants K18.1′ and K18.2/3′, were found only once and based on their low frequency they may be either mutations or true alleles. These variants are described in detail in the Examples below.

The unambiguous assignment of IDDMK_{1.2}22 to HERV-K18 had not been made previously for a number of reasons. For example, the published HERV-K18 LTR sequence (Ono et al. 1986) turned out to be identical to K18.2, which is as distantly related to the IDDMK_{1.2}22/K18.1 and K18.3 LTRs as it is from the HERV-K10 LTRs (˜7%). This explains why the IDDMK_{1.2}22/K18.1 LTR sequence originally appeared as an independent entity, identical neither to K18 nor to K10.

IDDMK_{1.2}22 encodes superantigen (SAg) activity within the envelope gene. The present inventors have established that the truncated and full-length HERV-K18 envelope alleles all encode superantigens (SAgs) with identical specificity.

The present inventors have also devised techniques for analyzing the polymorphism of the HERV-K18 locus in individuals. This has in turn provided a means for assessing the predisposition to disorders linked to the HERV-K18 locus, for example, disorders associated with the expression of SAg activity.

One particularly important disease which has been linked to IDDMK_{1.2}22 is insulin-dependent diabetes mellitus (IDDM). IDDM is an autoimmune disease due to the aggression of the β islets of Langerhans cells by islet-reactive T cells [Caillat-Zucman, 2000]. The existence of genetic control has long been known, since the disease involves a strong hereditary component. The problem is complicated by the multiplicity of predisposing genes, by the existence of protector genes, and by the relative low penetrance of predisposition. Additionally, the disease is heterogenous with variable rapidity of progression, exemplified by the difference in age onset. There may even exist particular subsets of patients in whom pathophysiology (and consequently the genetics) are clearly different from the bulk of other patients.

The search for predisposition genes has identified HLA (IDDM1) and insulin (IDDM2) genes as the major candidates associated with IDDM onset. The potential association of IDDMK_{1.2}22/HERV-K18 with IDDM, and particularly the discovery of the existence of allelic variation within HERV-K18 provides a further avenue of investigation for determination of predisposition to the disease.

A further genetic locus which may also play a role in favoring IDDM onset is the T cell receptor (TCR) locus. Genetic polymorphisms involving a large deletion within this locus (TCRβV) have been reported.

The present invention describes a novel method for identifying genetic predisposition to type I diabetes (IDDM) by analyzing the genetic polymorphism (genotyping) at at least one of 4 different loci. Two of these loci have not yet been linked to IDDM (HERV-K18 and TCRβV), whereas two other loci have already been identified as IDDM predisposition genes (IDDM1, the HLA class II region, and IDDM2 or INS, the insulin gene region).

Genotyping of the HERV-K18 locus for IDDM genetic predisposition is novel. The HERV-K18 locus and protein products is genetically and structurally distinct from the other HERV loci of the K family, such as HERV-K10. Genotyping for the TCR deletion in relation to genetic predisposition to IDDM is also novel. In addition, it is proposed that the combined value of polymorphism at locus HERV-K18 with polymorphism at one or more of the three other loci (TCRβV, IDDM1, and IDDM2) represents a significant improvement of the genotyping methodology for IDDM predisposition.

In the context of the present invention, the following terms signify:

<<human endogenous retrovirus>> (HERV): a retrovirus which is present in the form of proviral DNA integrated into the genome of all normal cells and is transmitted by Mendelian inheritance patterns. Such proviruses are products of rare infection and integration events of the retrovirus under consideration into germ cells of the ancestors of the host. Most endogenous retroviruses are transcriptionally silent or defective, but may be activated under certain conditions. Expression of the HERV may range from transcription of selected viral genes to production of complete viral particles, which may be infectious or non-infectious. Indeed, variants of HERV viruses may arise which are capable of an exogenous viral replication cycle, although direct experimental evidence for an exogenous life cycle is still missing. Thus, in some cases, endogenous retroviruses may also be present as exogenous retroviruses. These variants are included in the term <<HERV>> for the purposes of the invention. In the context of the invention, <<human endogenous retrovirus>> includes proviral DNA corresponding to a full retrovirus comprising two LTR's, gag, pol and env, and further includes remnants or <<scars>> of such a full retrovirus which have arisen as a results of deletions in the retroviral DNA. Such remnants include fragments of the full retrovirus, and have a minimal size of one LTR. Typically, the HERVs have at least one LTR, preferably two, and all or part of gag, pol or env.

HERV-K18: a full-length defective human endogenous retrovirus localized in Intron 1 of the CD48 gene on chromosome 1. The integration site of the HERV-K18 provirus in the large first CD48 intron has been found to be preserved in all individuals tested. The provirus is inserted in the opposite transcriptional direction to CD48.

Superantigen: a substance, normally a protein, of microbial origin that binds to major histocompatibility complex (MHC) Class II molecules and stimulates T-cell, via interaction with the Vβ domain of the T-cell receptor (TCR). SAgs have the particular characteristic of being able to interact with a large proportion of the T-cell repertoire, i.e. all the members of a given Vβ subset or <<family>>, or even with more than one Vβ subset, rather than with single, molecular clones from distinct Vβ families as is the case with a conventional (MHC-restricted) antigen. The superantigen is said to have a mitogenic effect that is MHC Class II dependent but MHC-unrestricted. SAgs require cells that express MHC Class II for stimulation of T-cells to occur.

Superantigen activity: <<SAg activity>> signifies a capacity to stimulate T-cells in an MHC-dependent but MHC-unrestricted manner. In the context of the invention, SAg activity can be detected in a functional assay by measuring either IL-2 release by activated T-cells, or proliferation of activated T-cells. Assays for the assessment and measurement of SAg activity are described in international patent application WO 99/05527, the content of which is hereby incorporated by reference.

Primer: in the context of the present invention, the term “primer” signifies a nucleic acid molecule having a length of 15 to 100 nucleotides, preferably 30 to 100 or 20 to 60 or 20 to 40 nucleotides, capable of specifically hybridizing to a template nucleic acid. Elongation of the primer by a DNA polymerase constitutes DNA synthesis. The primer is said to “correspond” to a given region of the DNA template target nucleic acid when it is identical or complementary to the said region (depending upon whether the primer is for forward or reverse amplification), and can thus hybridize to the template in conditions generally used in a nucleic acid amplification reaction. Hybridization conditions used in the context of the present invention are generally of high stringency, allowing primer-target binding to occur only when the primer and target sequences are exactly complementary or very nearly so, for example having no more than 2, and preferably no more than one mismatch, over a length of 20 nucleotides. The primers may include at one of their extremities, additional sequences which facilitate cloning, such as restriction sites, tags etc.

a <<human autoimmune disease>>: a polygenic disease characterized by the selective destruction of defined tissues mediated by the immune system. Epidemiological and genetic evidence also suggests the involvement of environmental factors.

cells which <<functionally express>> SAg: cells which express SAg in a manner suitable for giving rise to MHC-dependent, MHC-unrestricted T-cell stimulation in vitro or in vivo. This requires that the cell be MHC II⁺ or that it has been made MHC II⁺ by induction by agents such as IFN-γ.

### BRIEF DESCRIPTION OF THE FIGURES

FIG. 1: Genomic organization and polymorphism of the HERV-K18 alleles.

FIG. 2: Allelic variants of the HERV-K18 ENV protein (SEQ ID NO: 1). Alleles actually found in analyzed populations, as well as further potential alleles based on all possible nucleotide variations at the polymorphic sites, are shown.

| | |
|---|---|
| Xaa97: | Tyr, Cys, Phe, Ser |
| Xaa154: | Trp, Leu, Ser, Stop |
| Xaa272: | Val, Ile, Leu |
| Xaa348: | Val, Ile, Leu, Phe |
| Xaa534: | Val, Ile, Leu, Phe |

FIG. 3: Superantigen activity of the HERV-K18 alleles. The env protein encoded by the HERV-K18 alleles display superantigen activity and specifically stimulate T cells expressing the Vβ7 and Vβ13.1 T cell receptors. A20 cells expressing HERV-K18.1 and -K18.3 specifically stimulated proliferation of T cells expressing the Vβ7 T cell receptor (FIG. 3A). A20 cells expressing HERV-K18.2 also stimulated proliferation of T cells expressing the Vβ7 T cell receptor (data not shown). In addition, A20 cells expressing HERV-K18.1 specifically stimulated IL-2 release from T cells expressing the Vβ13.1 T cell receptor, but not T cells expressing control T cell receptors the Vβ8 T cell receptor (FIG. 3B).

FIG. 4: Nucleotide sequences of K18-1 ENV ((SEQ ID NO: 2; FIG. 4A), K18-2 ENV (SEQ ID NO: 3; FIG. 4B) and K18-3 ENV (SEQ ID NO: 4; FIG. 4C). The start codon ATG, and the stop codons TGA and TAG are shown in bold type.

FIG. 5: 5′ Untranslated region (UTR) of HERV-K18 ENV (SEQ ID NO: 6). This sequence is unique to Herv K-18 and is common to all alleles. It is particularly suitable as a primer for amplification of the ENV region.

FIG. 6: Amino acid sequences of the HERV-K18 ENV alleles: K18.1 (SEQ ID NO: 7; FIG. 6A), K18.2 (SEQ ID NO: 8; FIG. 6B), K18.3 (SEQ ID NO: 9; FIG. 6C), K18.2/3′ (SEQ ID NO: 10; FIG. 6D). Amino acid variations arising from SNP polymorphism are boxed.

FIG. 7: Amino acid sequence alignment of the HERV-K18 ENV alleles (SEQ ID NO: 7-9).

FIG. 8: Nucleotide sequence alignment of the HERV-K18 alleles of the ENV coding region (SEQ ID NOS: 11-14).

FIG. 9: Nucleotide sequences of LTR regions of HERV-K18 (3LTR K18-1; SEQ ID NO: 15; FIG. 9A; 3LTR K18-2; SEQ ID NO: 16; FIG. 9B; 3LTR K18-3; SEQ ID NO: 17; FIG. 9C; 5LTR K18-1; SEQ ID NO: 18; FIG. 9D; 5LTR K18-2; SEQ ID NO: 19; FIG. 9E; 5LTR K18-3; SEQ ID NO: 20; FIG. 9F: 3LTR K18-1 I insert; SEQ ID NO:21; FIG. 9G)

FIG. 10: LTR alignments. % identities are the following:

3 K18-1 against 3 K18-2: Identities=971/975 (99%)

3 K18-1 against 3 K18-3: Identities=968/975 (99%)

3 K18-1 against 5 K18-1: Identities=930/969 (95%)

3 K18-1 against 5 K18-2: Identities=930/970 (95%)

3 K18-1 against 5 K18-3: Identities=933/970 (96%)

FIG. 11: Polymorphism analysis of the TCRβV deletion locus. These primers (SEQ ID NOS: 22-25) are listed as examples. “X” corresponds to deletion region.

FIG. 12: Duplex PCR with 200 ng genomic DNA as template. Cycling conditions were touchdown annealing temperatures from 68 to 60° C. during the first 10 cycles, followed by 30 cycles at 60° C. The molar ratio of external to internal primers was critical: 30 pmol external primers (5′- and 3′-TCR) and 10 pmol internal primers (5′- and 3′-V7.2). E=positive control of the deletion allele (size 1400 bp). I=positive control of the wt allele (size 710 bp). EI=duplex PCR performed on a human sample. The gels show that both samples tested were wt since the size of the fragment is 710 bp.

FIG. 13: Sequence of Intron I of CD48 in the regions flanking the integration site of HERV-K18 (3′ LTR) (SEQ ID NO: 26). The numbering is that used in GenBank accession no. AL 121985. HERV-K18, integrated in an inverse orientation. The sequence of FIG. 13 is shown in the direction of transcription of CD48.

Specific regions are positioned as follows:

10001-11781: CD48 intron (5′ portion);

11782: boundary between CD48 intron (5′ portion) and HERV-K18 (3′end of 3′ LTR);

11782 to 12755: HERV-K18 3′0LTR;

12793-12795: STOP codon of HERV-K18 ENV;

14473-14475: START codon of HERV-K18 ENV;

14537-14556: primer FPYRO (reversed)

14647-14649: STOP codon of HERV-K18 POL.

FIG. 14: Schematic representation of the genomic organization of the HERV-K18 locus with indications of examples of suitable primers for the genotyping of ENV and/or LTR regions. Double arrows ( [symbol] , [symbol] ) indicate direction of transcription of the CD48 gene and of the HERV-K provirus.

FIG. 15: Sequence of the HERV-K18 locus (SEQ ID NO: 42), extending from position 13982, situated within the CD48 intron (3′ portion) through the full HERV-K18 insert to the 5′ portion of the CD48 intron (5′ end). The sequence of FIG. 15 (SEQ ID NO: 42) is shown in the direction of transcription of HERV-K18 (CD48 is therefore inverted). The illustrated sequence of HERV-K18 is allele K18.3, but the illustrated genomic organization is identical for all alleles.

Specific regions are positioned as follows:

Positions 13982 to 14744: CD48 intron (3′ portion);

Positions 14745 to approximately 15715: 5′ LTR of HERV-K18;

Positions 21121 to 21287 (bold type): untranslated region (UTR) of HERV-K18 ENV;

Positions 21207 to 21226 (boxed and shaded): primer FPYRO;

Position 21288 to 21290 (boxed): initiation codon of HERV-K18 ENV;

Position 21288 to 22970: coding sequence of HERV-K18 ENV;

Position 21747 to 21749 (boxed): TGG codon which is replaced by TAG STOP codon in HERV-K18.1 ENV

Positions 22946 to 22962 (boxed): sequence contained within primer K18LTR;

Position 22968 to 22970 (boxed): stop codon of HERV-K18 ENV;

Positions 23008 to 23982 (bold type): 3′LTR of HERV-K18;

Positions 23975 to 24000 (boxed and shaded): primer K18FLR1 (reversed);

Positions 23983 to 24549: CD48 intron (5′ end, junction with HERV-K18 3′LTR).

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

A first aspect of the invention relates to the previously unknown polymorphic variants of the HERV-K18 provirus, both at the nucleic acid level and at the protein level.

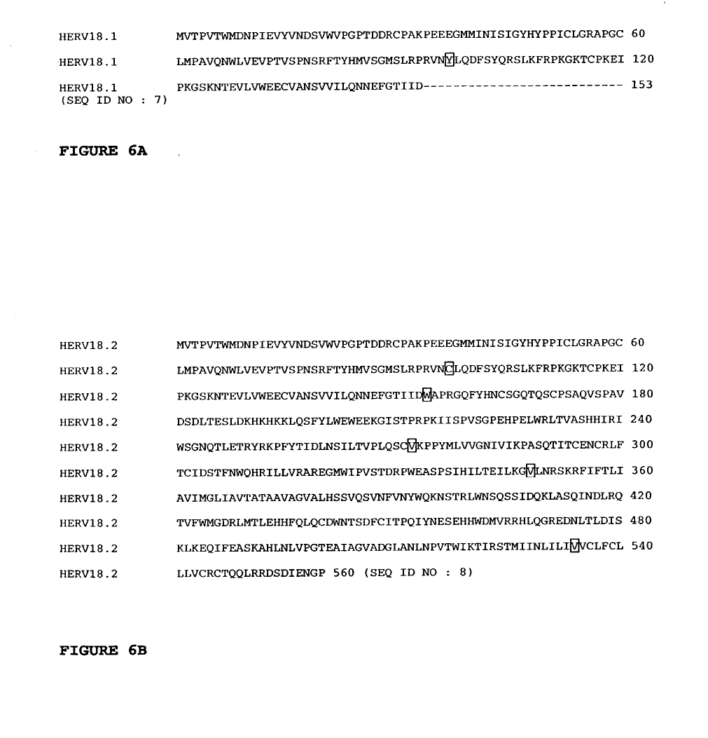

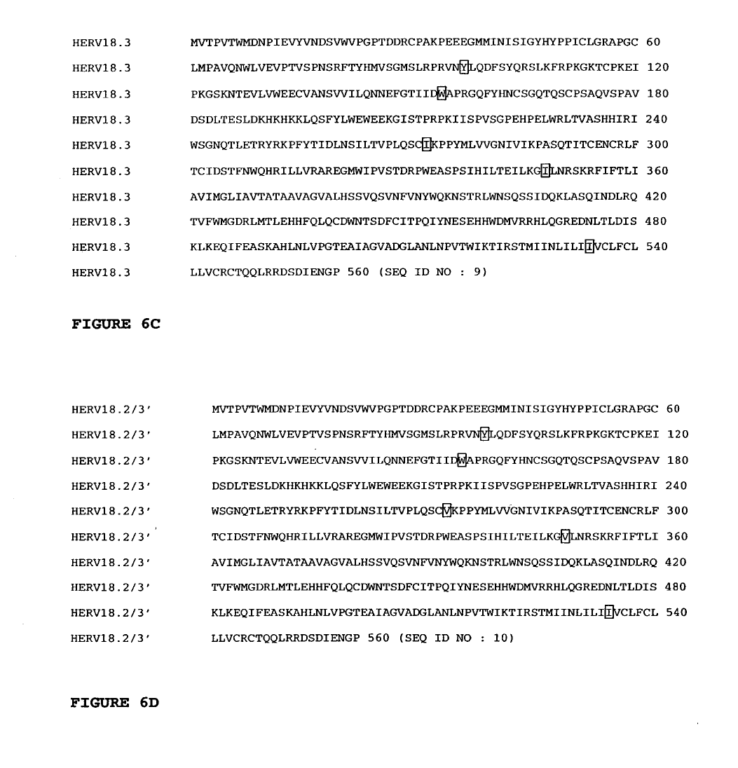

More specifically, this aspect of the present invention relates to the expression products of the different HERV-K18 ENV alleles, and to fragments of these expression products. Generally speaking, the variants have from 98.5 to 99.9% identity, preferably 99.0% to 99.9% identity with HERV-K18.2 ENV (SEQ ID NO: 8; shown in FIG. 6B), and preferably, but not necessarily, have a length of 560 amino acids. The % identity is expressed with respect to the full length 560 K18.2 sequence (SEQ ID NO: 8).

Also included in the invention are variants of the truncated HERV ENV K18.1 protein (SEQ ID NO: 7), having from 98.0% to 99.9% identity with the protein illustrated in FIG. 6A. The % identity is expressed with respect to the truncated 153 K18.1 sequence of FIG. 6A (SEQ ID NO: 7). Such variants preferably, but not necessarily, have a length of 153 amino acids.

Particularly preferred are the expression products of the alleles HERV-K18 env gene having at least 99.5% identity, for example 99.6, 99.7, 99.8 or 99.9% identity with the proteins illustrated in FIGS. 6A and 6B (SEQ ID NO: 7-8).

Variants may have for example one, two, three, four or five amino acid substitutions with respect to the sequences shown in FIGS. 6A and B. More particularly, the invention includes the following variants:

variants of K18-1: having one, two or three single amino acid substitutions/deletions/insertions with respect to the sequence shown in FIG. 6A (SEQ ID NO: 7);

variants of K18-2: having from one to five single amino acid substitutions/deletions/insertions with respect to the sequence shown in FIG. 6B (SEQ ID NO: 8). A preferred length is 560 amino acids. Particularly preferred are variants having one, two or three amino acid substitutions compared to K18-2.

According to a preferred embodiment, at least one of the amino acid substitutions, deletions and/or insertions with respect to the K18.1 and K18.2 alleles occurs at a position chosen from at least one of positions 97, 154, 272, 348, and 534 as illustrated in FIGS. 6A and 6B (SEQ ID NO: 7-8).

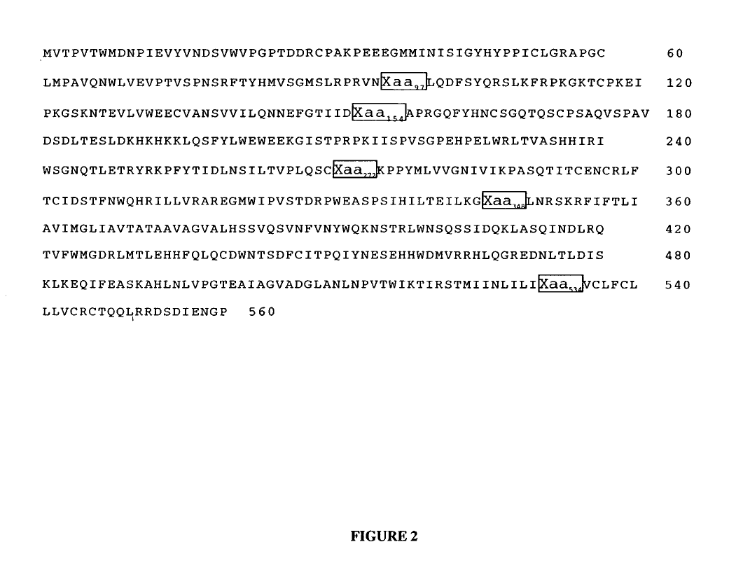

According to a further preferred embodiment, the protein comprises or consists of the amino acid sequence illustrated in FIG. 2 (SEQ ID NO: 1), wherein Xaa₉₇, Xaa₁₅₄, Xaa₂₇₂, Xaa₃₄₈, Xaa₅₃₄ are chosen from the following amino acids:

| | |
|---|---|
| Xaa₉₇: | Tyr, Cys, Phe or Ser |
| Xaa₁₅₄: | Trp, Leu, Ser or, Stop |
| Xaa₂₇₂: | Val, Ile or Leu |
| Xaa₃₄₈: | Val, Ile, Leu or Phe |
| Xaa₅₃₄: | Val, Ile, Leu or Phe |

with the proviso that Xaa₉₇ is not Tyr when Xaa₁₅₄ is STOP, and that Xaa₉₇ is not Cys when Xaa₁₅₄ represents Trp and each of Xaa₂₇₂, Xaa₃₄₈, Xaa₅₃₄ represent Val.

Table I below summarizes the different amino acids which may occur at positions 97, 154, 272, 348, and 534 of the FIG. 2 sequence (SEQ ID NO: 1; using the single letter amino acid code). The invention includes proteins having any of the possible combinations arising from these different possibilities, except the known HERV-K18.1 and HERV-K18.2 proteins. For example, an allele having a Cys at position 97 and a Stop at position 154, or an allele having Cys at position 97, Trp at position 154, Ile at positions 272 and 348, and Val at position 534, are included.

TABLE I
HERV K18 Polymorphic sites: ENV

| | 97 | | 154 | | 272 | | 348 | | 534 | |
|---|---|---|---|---|---|---|---|---|---|---|
| | | Amino | | Amino | | Amino | | Amino | | Amino |
| | Nt | acid | Nt | acid | Nt | acid | Nt | acid | Nt | acid |
| K18.1 | TAT | Y | TAG | STOP | CTA | — | GTT | — | GTT | — |
| K18.2 | TGT | C | TGG | W | GTA | V | GTT | V | GTT | V |
| K18.3 | TAT | Y | TGG | W | ATA | I | ATT | I | ATT | I |
| K18.2/3 | | Y | | W | | V | | V | | I |
| Potential | TTT | F | TTG | L | TTA | L | CTT | L | CTT | L |
| further alleles | TCT | S | TCG | S | | | TTT | F | TTT | F |

Examples of particular variants are proteins comprising or consisting of HERV ENV K18.3 and HERV-ENV K18.2/3′ illustrated in FIGS. 6C and 6D respectively (SEQ ID NOs: 8 and 9).

According to a particular embodiment, the proteins of the invention exhibit superantigen (SAg) activity. Assays for the assessment and measurement of such activity are described in international patent application WO 99/05527, the content of which is hereby incorporated by reference. For example, the capacity of a protein of the invention to exhibit SAg activity can be detected by carrying out a functional assay in which MHC class II+ cells expressing the protein (either a biological fluid sample containing MHC class II+ cells, or MHC Class II+ transfectants) are contacted with cells bearing one or more variable β-T-receptor chains and detecting preferential proliferation of a Vβ subset.

If a biological sample is used, it is typically blood and necessarily contains MHC class II+ cells such as B-lymphocytes, monocytes, macrophages or dendritic cells which have the capacity to bind the superantigen and enable it to elicit its superantigen activity. MHC class II content of the biological sample may be boosted by addition of agents such as IFN-gamma.

The biological fluid sample or transfectants are contacted with cells bearing the Vβ-T receptors belonging to a variety of different families or subsets in order to detect which of the Vβ subsets is stimulated by the putative SAg, for example V-β2, 3, 7, 8, 9, 13 and 17. Within any one V-β family it is advantageous to use V-β chains having junctional diversity in order to confirm superantigen activity rather than nominal antigen activity.

T-cell hybridoma bearing defined T-cell receptor may also be used in the functional or cell-based assay for SAg activity. An example of commercially available cells of this type are given in B. Fleischer et al. Infect. Immun. 64, 987-994, 1996. Such cell-lines are available from Immunotech, Marseille, France. According to this variant, activation of a particular family of V-β hybridoma leads to release of IL-2. IL2 release is therefore measured as read-out using conventional techniques.

According to the present invention, the different allelic variants of the ENV protein have SAg activity specific for Vβ7 and/or Vβ13 chains. Preferably, both Vβ7 and Vβ13 activity is present, particularly Vβ13.2

The invention also relates to peptide fragments of the allelic variants of HERV-K18 ENV described above.

Preferably, such a protein fragment or peptide comprises or consists of a fragment of the protein illustrated in FIG. 2, said fragment having a length of 6 to 556 amino acids, and includes the portion spanning at least one of positions 154, 272, 348, 534 of the sequence illustrated in FIG. 2, wherein Xaa₉₇, Xaa₁₅₄, Xaa₂₇₂, Xaa₃₄₈, Xaa₅₃₄ are chosen from the following amino acids:

| | |
|---|---|
| Xaa₉₇: | Tyr, Cys, Phe, Ser |
| Xaa₁₅₄: | Trp, Leu, Ser, Stop |
| Xaa₂₇₂: | Val, Ile, Leu |
| Xaa₃₄₈: | Val, Ile, Leu, Phe |
| Xaa₅₃₄: | Val, Ile, Leu, Phe |

with the proviso that Xaa₉₇ is not Tyr when Xaa₁₅₄ is STOP, and that Xaa₉₇ is not Cys when Xaa₁₅₄ represents Trp and each of Xaa₂₇₂, Xaa₃₄₈, Xaa₅₃₄ represent Val.

Further examples of protein fragments of the invention comprise or consist of a fragment of the protein illustrated in FIG. 6C or FIG. 6D. Such fragments have a length of 6 to 556 amino acids, including the portion spanning at least one of positions 154, 272, 348, 534 of the sequence illustrated in FIG. 6C (SEQ ID NO: 9) or FIG. 6D (SEQ ID NO: 10).

Preferably, the protein fragment or peptide has a length of 10 to 300 amino acids, for example 12 to 200 amino acids, such as 15 to 150, or 20 to 100, or 15 to 25 amino acids.

Examples of preferred peptides comprise or consist of amino acids 96-155, 90-300, 100-200, 150-560, 200-400, 300-540 of HERVK-18.3 (SEQ ID NO: 9; FIG. 6C) or HERVK-18.2/3′ (SEQ ID NO: 10; FIG. 6D).

According to a particularly preferred embodiment, the protein fragment or peptide derived from the ENV allelic variant described above may or may not have Superantigen (SAg) activity. Indeed, depending upon the length and the composition of the fragment, the SAg activity of the parent ENV molecule may be either lost or conserved. Preferably, the fragments exhibit superantigen activity specific for Vβ7 and/or Vβ13 chains. This may be the case for fragments having a length of at least 50, and preferably at least 100 amino acids. Shorter peptides, or those derived from the C-terminal end of ENV (i.e. beyond position 154, for example beyond position 300) may be devoid of superantigen activity, for example those having a length of between 10 to 40 amino acids. Generally speaking, such peptides have no substantial Vβ7 and/or Vβ13 SAg activity.

The invention also relates to the nucleic acid molecule encoding the proteins and peptides of the invention.

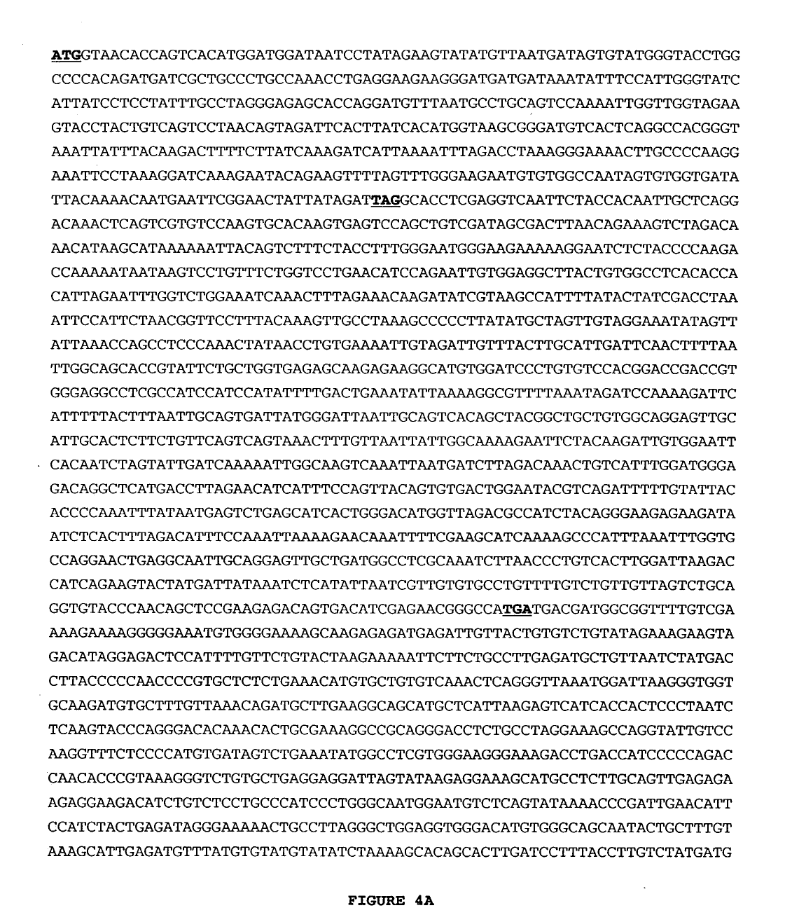

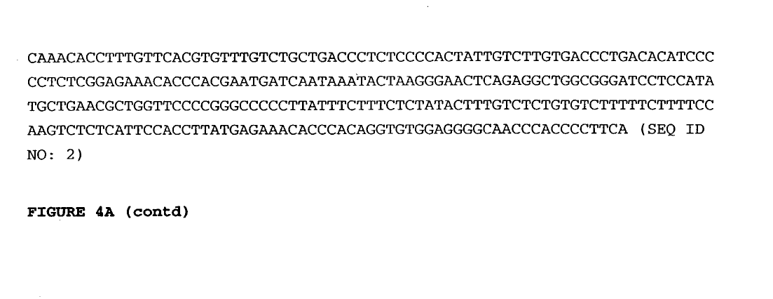

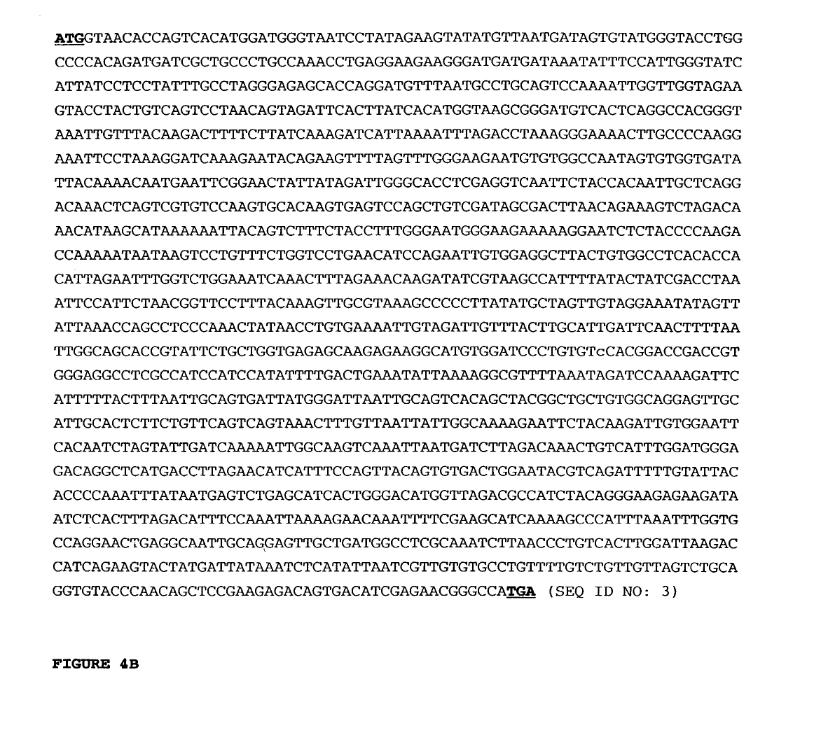

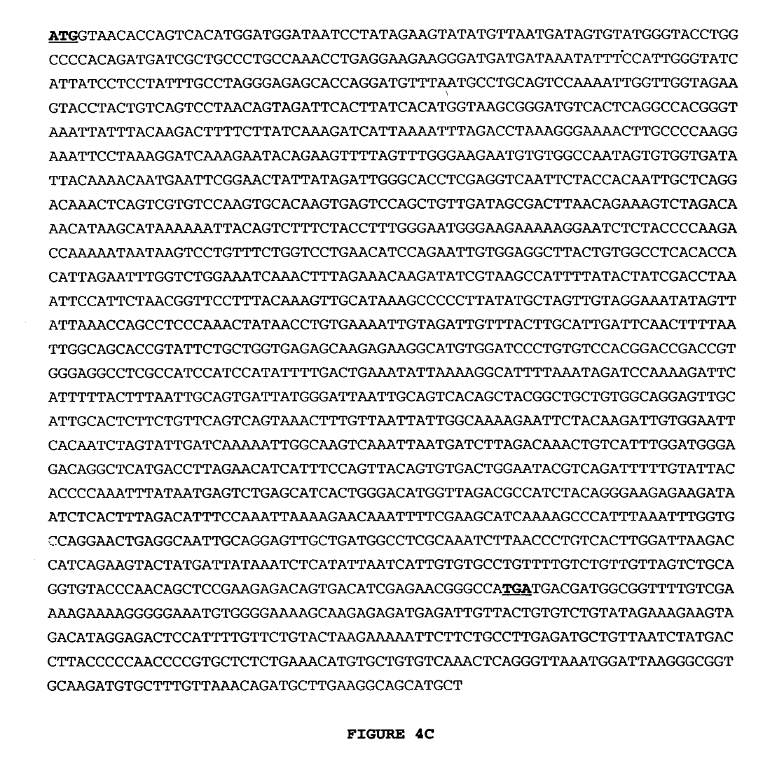

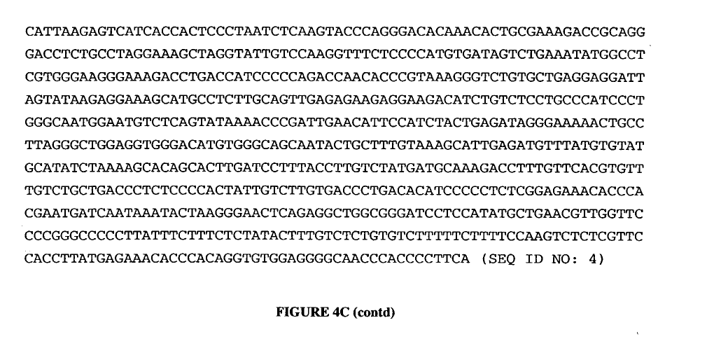

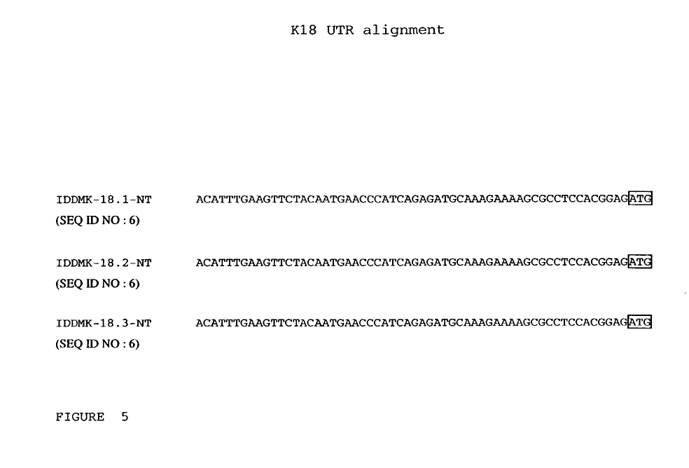

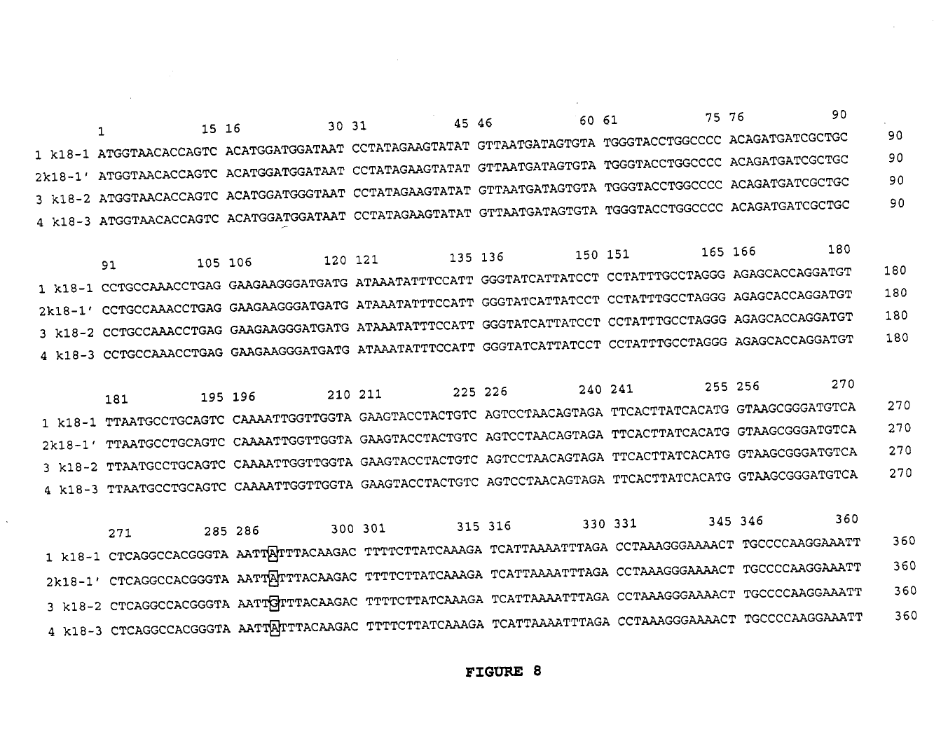

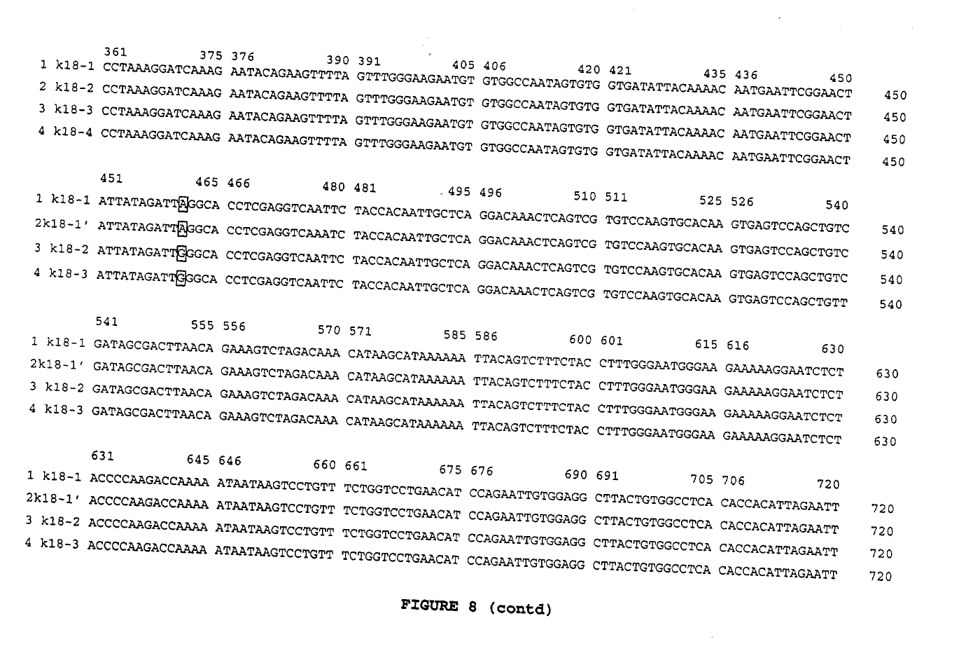

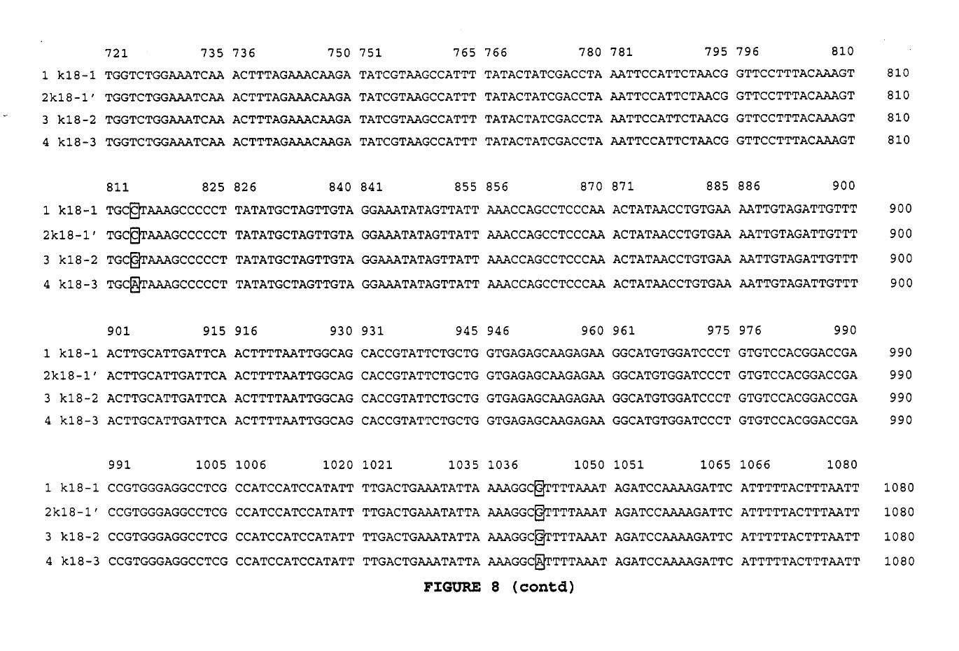

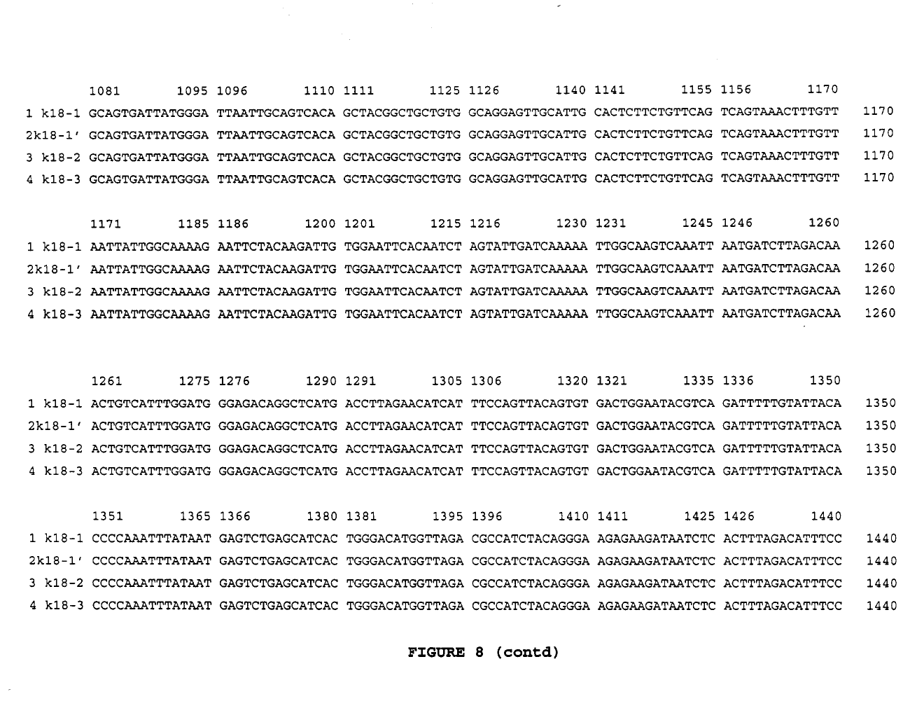

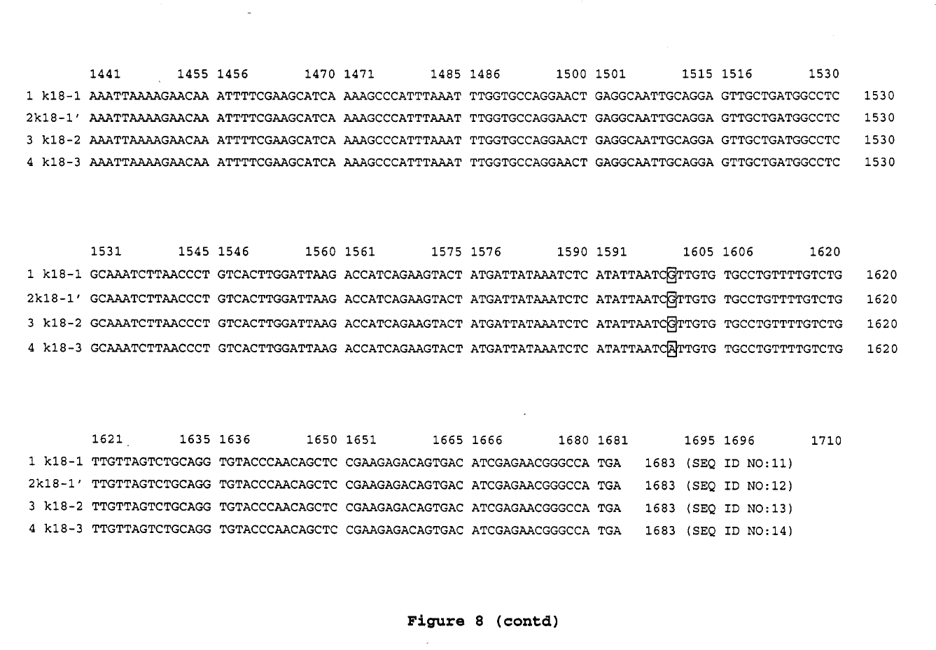

Particularly preferred nucleic acid molecules comprise or consist of a sequence having from 1 to 15 nucleotide insertions, substitutions or deletions with respect to the K18.2 nucleic acid sequence illustrated in FIG. 8 and FIG. 4B, for example 1 to 9 insertions, substitutions or deletions. Preferably the nucleotide changes are single nucleotide substitutions.

Preferred examples are nucleic acid molecules comprising or consisting of the K18.3 nucleic acid sequence illustrated in FIG. 8 (SEQ ID NO: 13), and nucleic acid molecules comprising or consisting of a sequence encoding HERV ENV K18.2/3′ illustrated in FIG. 6D (SEQ ID NO: 10).

Fragments of the nucleic acids encoding the ENV alleles also form part of the invention. Such fragments generally have from 16 to 1668 nucleotides and include the nucleotides encoding the amino acids at positions 97, 154, 272, 348, 534 of the sequence illustrated in FIG. 6C or FIG. 6D. Preferably, these nucleic acid fragments have a length of from 20 or 30 to 900 nucleotides, for example 60 to 500 nucleotides, such as 75 to 300 nucleotides.

The invention also includes nucleic acid molecules having a sequence complementary to the ENV-encoding sequences and their fragments. Such complementary sequences are useful as antisense oligonucleotides, or as primers in amplification reactions, or as probes.

The nucleic acid molecules of the invention may be DNA or RNA.

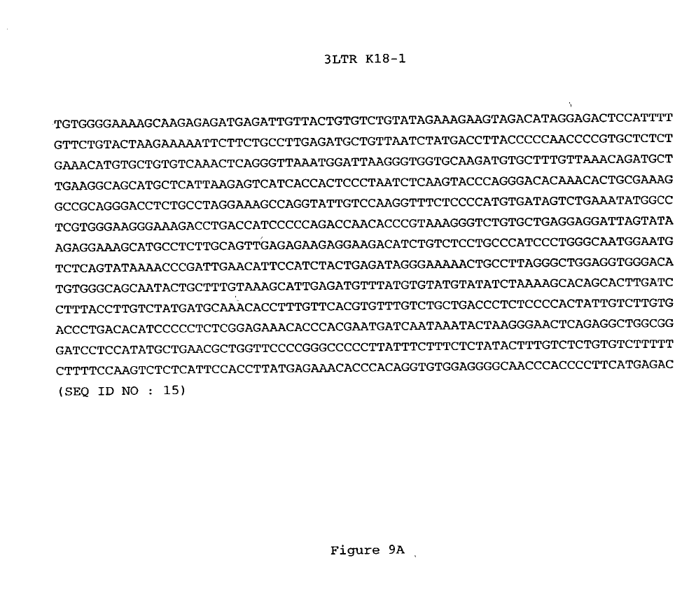

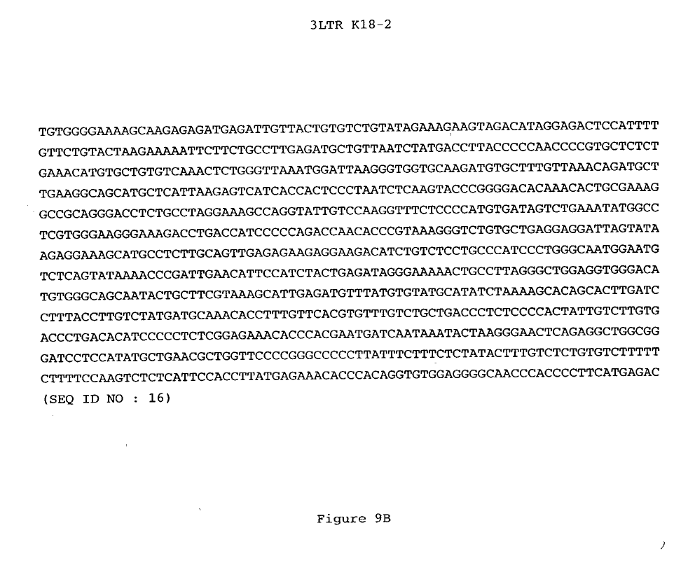

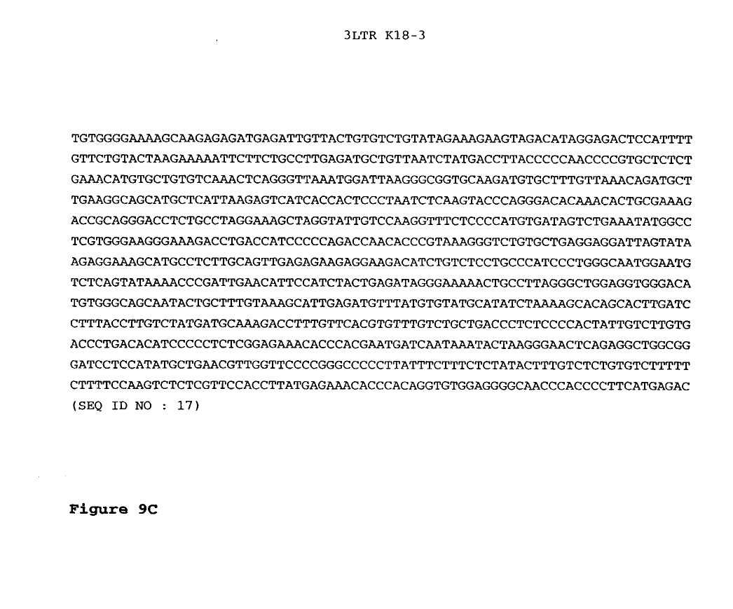

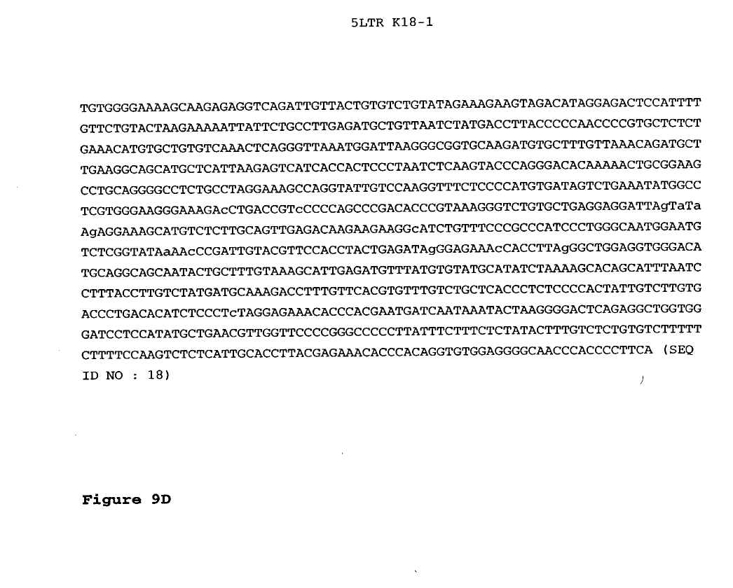

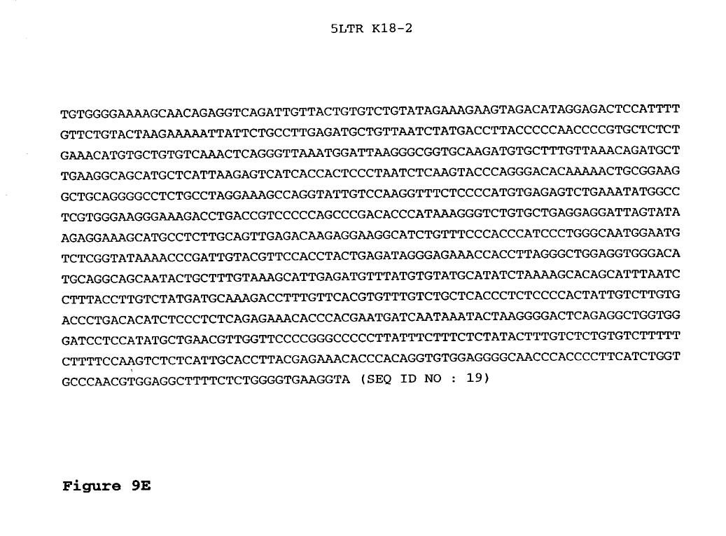

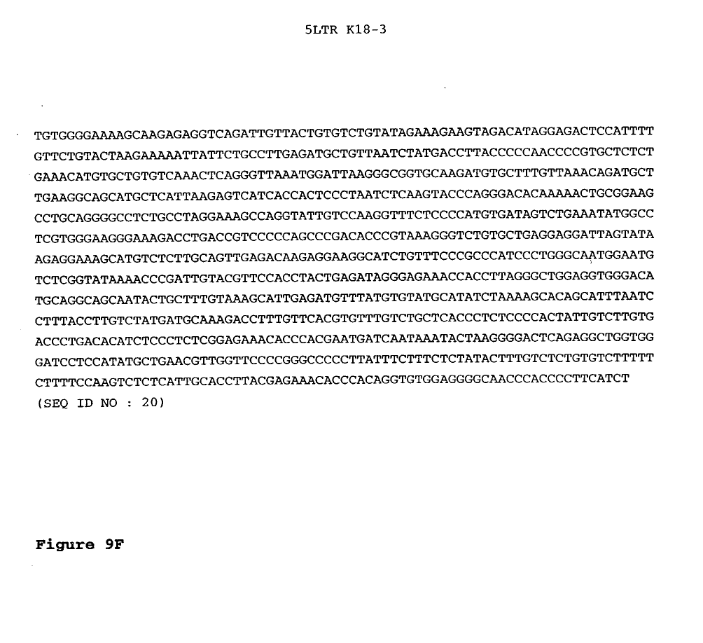

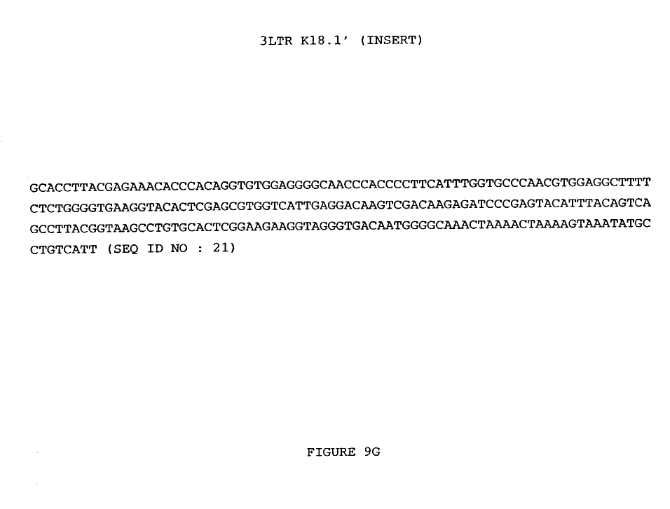

According to a particularly preferred embodiment, the invention relates to the 5′ and 3′ LTR regions of the HERV-K18 provirus alleles. These regions have been found to be polymorphic. Particularly preferred examples are nucleic acid molecules comprising or consisting of the sequence illustrated in FIG. 9A (SEQ ID NO: 15; 3′ LTR K18.1), or FIG. 9C (SEQ ID NO: 17; 3′ LTR K18.3). A further example are nucleic acid molecules comprising or consisting of the sequence illustrated in FIG. 9D (SEQ ID NO: 18; 5′ LTR K18.1) or FIG. 9F (SEQ ID NO: 20; 5′ LTR K18.3).

Further examples are variants of the 3′LTR K18.2 (SEQ ID NO: 16) and 5′LTR K18.2 (SEQ ID NO: 19) illustrated in FIGS. 9B and 9E respectively. Such variants exhibit between 99.0 and 99.9% identity, for example between 99.5 and 99.85% with the illustrated sequences, with respect to the full length K18.2 LTR sequences.

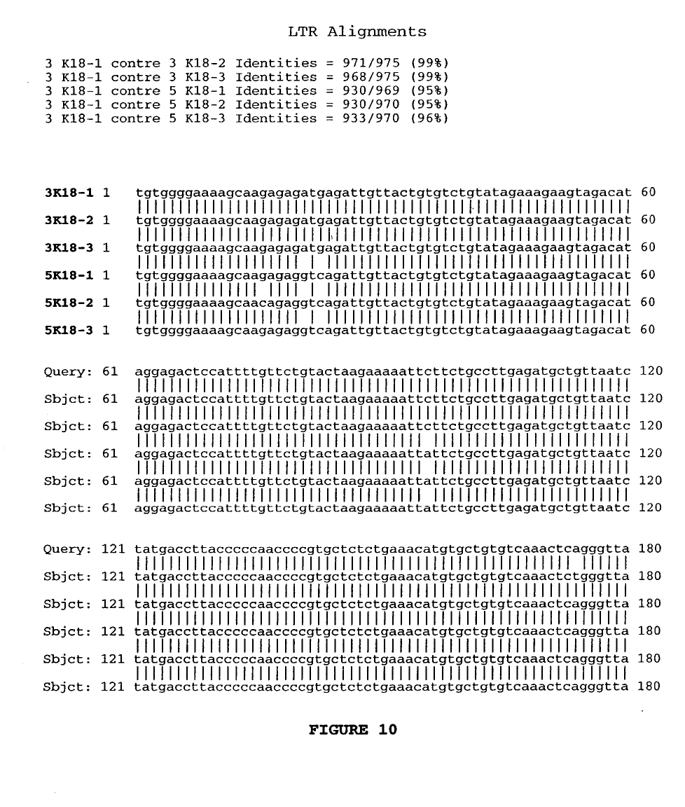

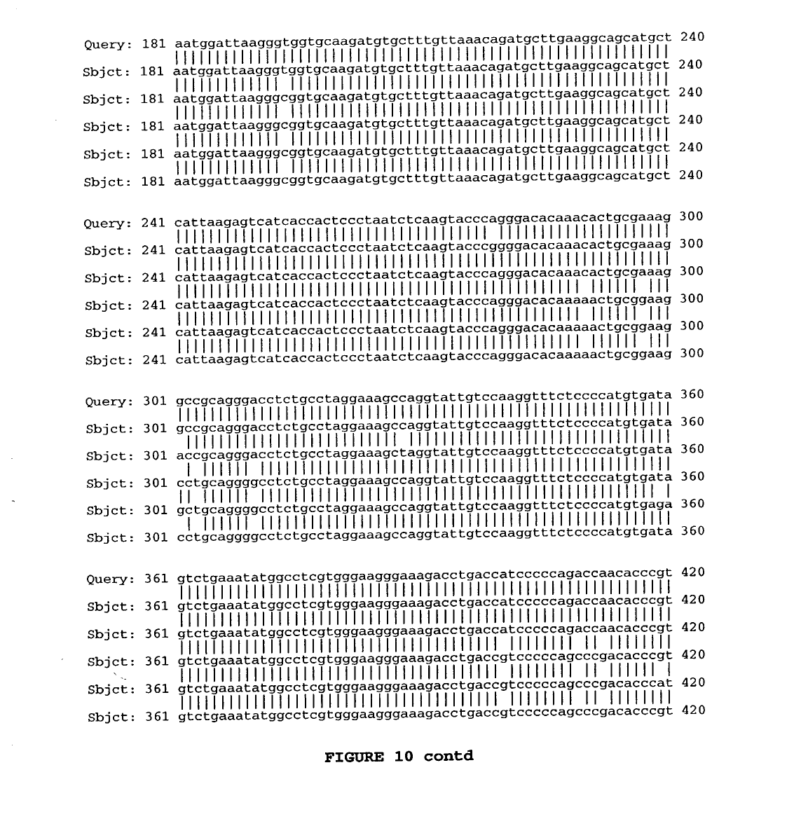

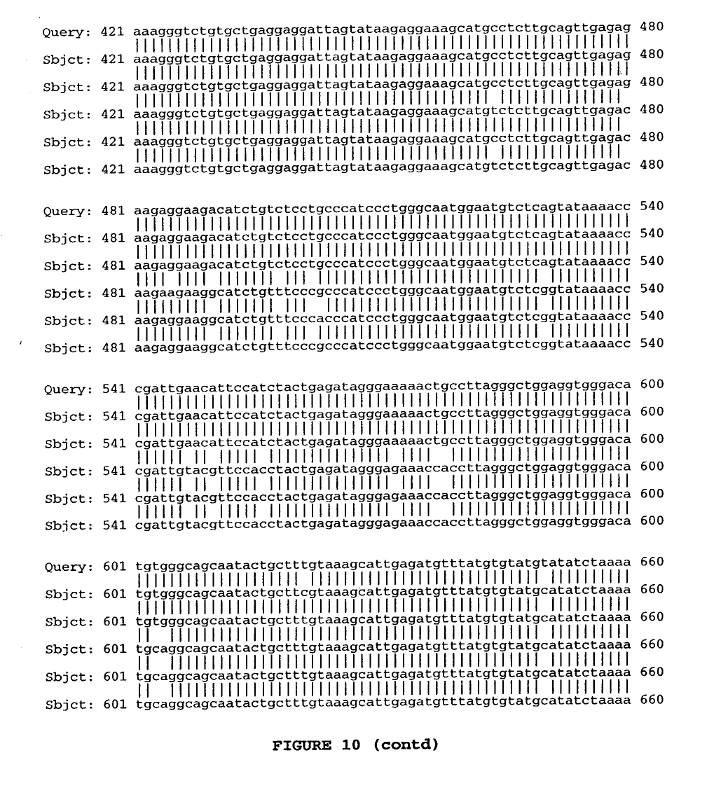

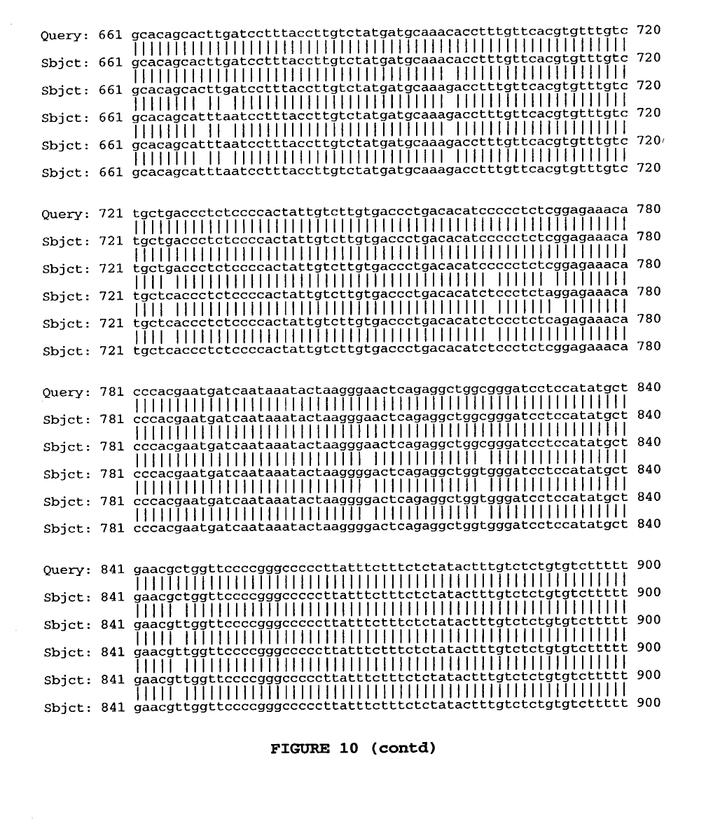

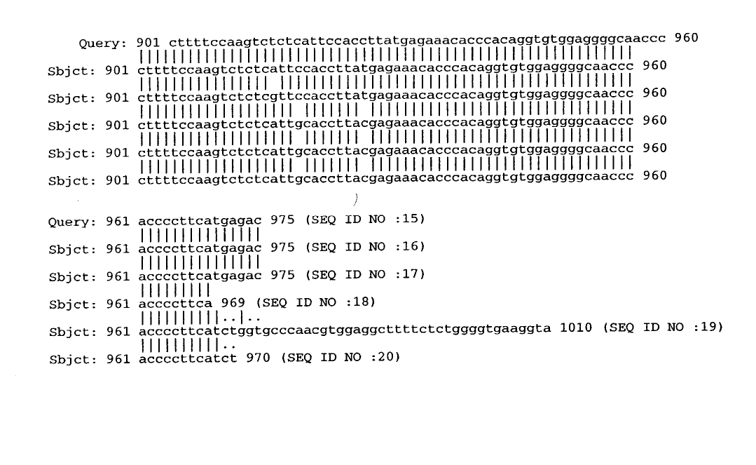

The invention also relates to nucleic acid molecules derived from the LTRs which are suitable for use as a primer in a nucleic acid amplification reaction, preferably for amplifying a portion of the LTR. Such molecules have a length of approximately 30 to 300 nucleotides, for example 30 to 100, and have a sequence common to all sequences aligned in FIG. 10, or a sequence complementary thereto. Preferably, the primers have a sequence identical to, or complementary to, the 3′ LTR sequences illustrated in FIG. 10 between positions 1-173, 195-278, 329-620, 651-698, 700-845. Also preferred are primers having a sequence identical to, or complementary to, the 5′ LTR sequences illustrated in FIG. 10 between positions 20-300, 305-460, 505-770.

The invention also relates to antibodies specifically recognizing a protein or peptide of the invention. The antibodies may be polyclonal or monoclonal. Particularly preferred are antibodies capable of distinguishing between the different alleles of HERV-K18 ENV. Such antibodies are raised for example to peptides having from 10 to 100 amino acids, or more, characteristic of the different alleles, for example:

HERV K18.1 ENV: C-terminus (e.g. amino acids 140-153 of SEQ ID NO: 7)

HERV K18.2 ENV: amino acids 270-280, 340-350 of SEQ ID NO: 8

HERV K18.3 ENV: amino acids 528-538 of SEQ ID NO: 9

Such differential antibodies can also be used in the determination of genotypes of individuals expressing the ENV protein.

A major aspect of the present invention concerns a method for the identification of HERV K-18 alleles in human individuals. The method comprises i) a first step of analysis of at least one of the polymorphic regions of HERK-K18 in both chromosomes of an individual, particularly ENV and/or the 5′ or 3′ LTRs to determine the sequence of said region, ii) followed by assignment of a genotype on the basis of the sequence identified in the polymorphic region.

The step of analysis of the genomic DNA of an individual can be carried out by any suitable method. Particularly preferred is specific amplification of the ENV or LTR region, for example using PCR techniques, followed by analysis of the sequence of the amplified region or part thereof, to determine the polymorphic form of the individual under examination. The sequence analysis can be implemented by direct sequencing, restriction length polymorphism (RFLP), single mismatch PCR, primer extension techniques, hybridization of specific probes etc.

A preferred method of the invention thus comprises 3 steps: A) PCR amplification of human DNA, B) analysis of single nucleotide polymorphisms, and C) recording of the genotype corresponding to the HERV-K18 alleles.

Amplification of genomic DNA is carried out using suitable primers chosen to allow amplification of at least a portion of the env region of HERV-K18, or at least a portion of the 5′ or 3′ LTR. The minimum portion of the ENV region which should be amplified is the portion encoding amino acids 97 to 154 of ENV as illustrated in FIGS. 6A, 6B, 6C, or 6D of SEQ ID NO: 7-10). A preferred portion for amplification comprises both ENV and the adjacent 3′LTR.

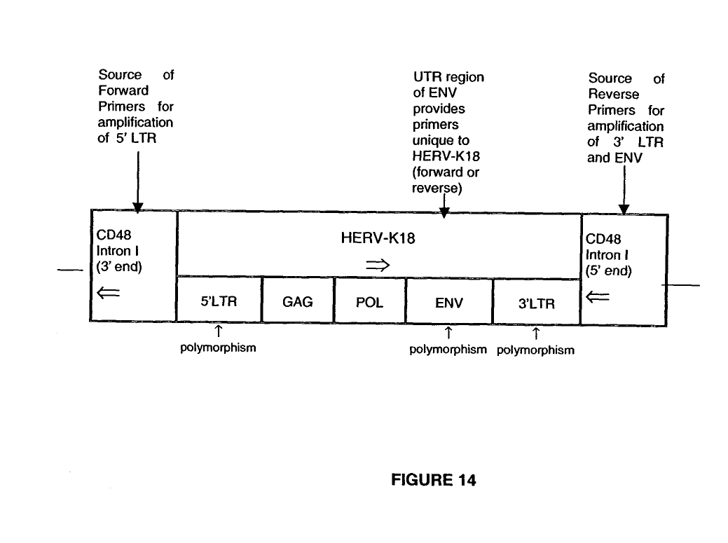

Preferably, at least one of the two primers used for amplification of the polymorphic regions is unique to the HERV-K18 locus. The HERV-K18 provirus is integrated into the human genome in the first intron of the CD48 gene, in an inverted orientation. The sequences of Intron I of the CD48 gene, and also Exons 1 and 2, are unique to this locus and therefore provide a source of suitable sequences for use as primers. The sequence of the CD48 gene (SEQ ID NO: 26; See GenBank accession no. AL 121985). Intron I extends from nucleotides 122 to 26613 of the CD48 gene (numbering starting at the initiation codon). FIG. 14 provides a schematic representation of the genomic organization of the locus with indications of examples of suitable primers.

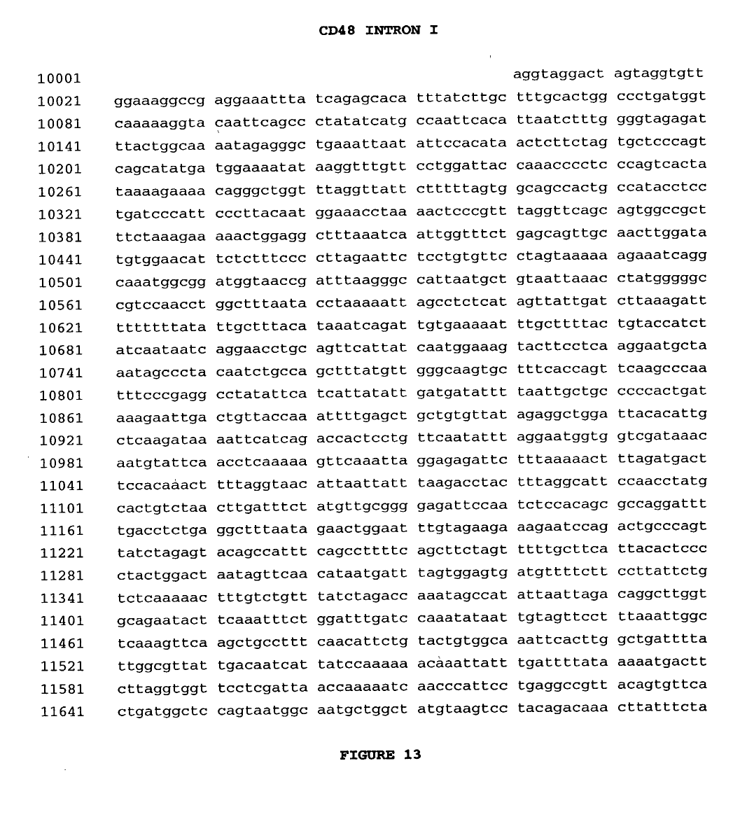

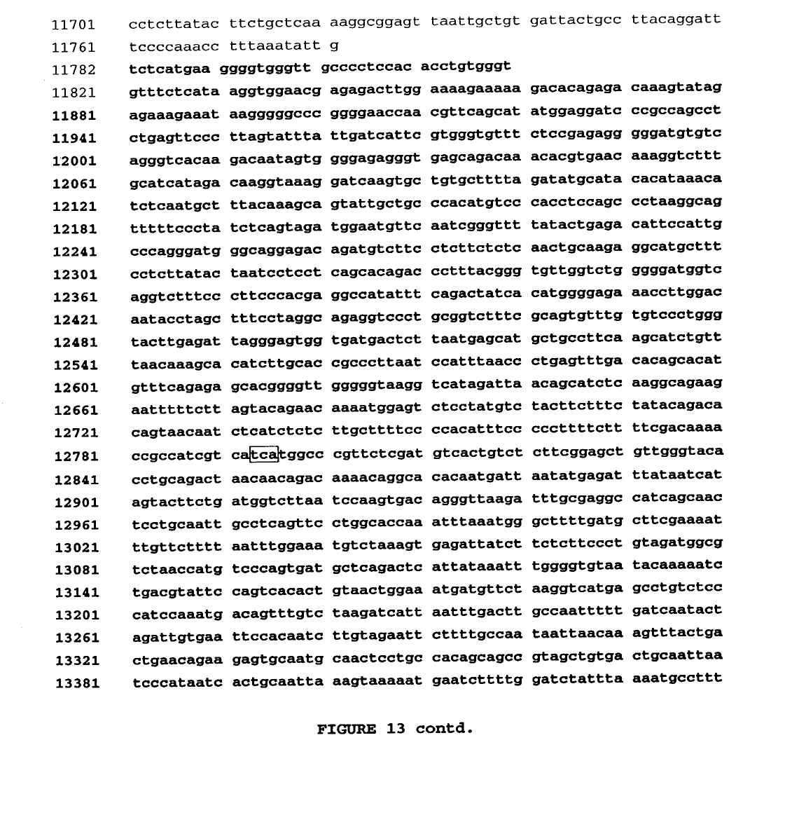

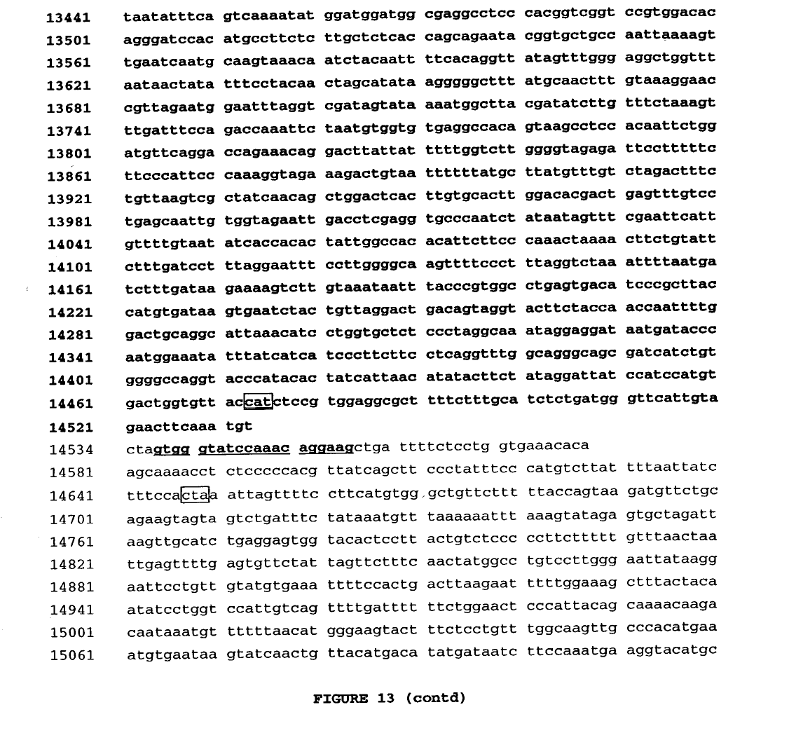

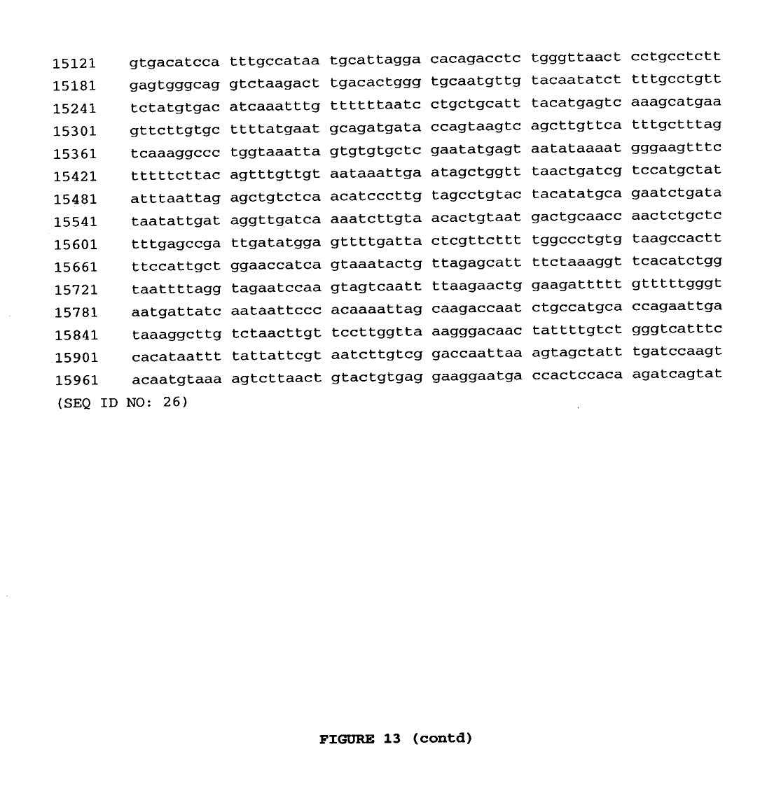

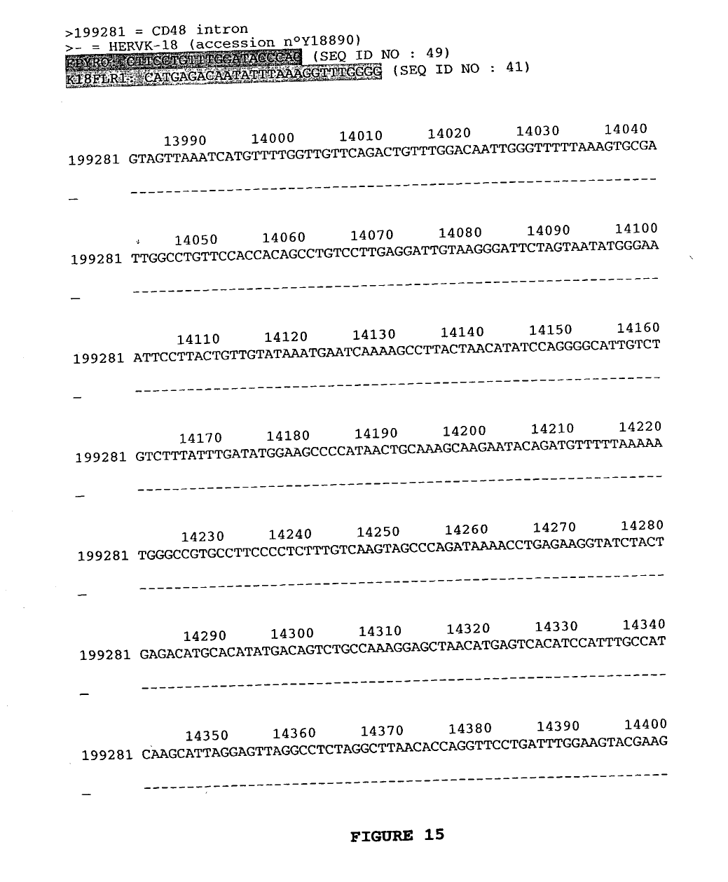

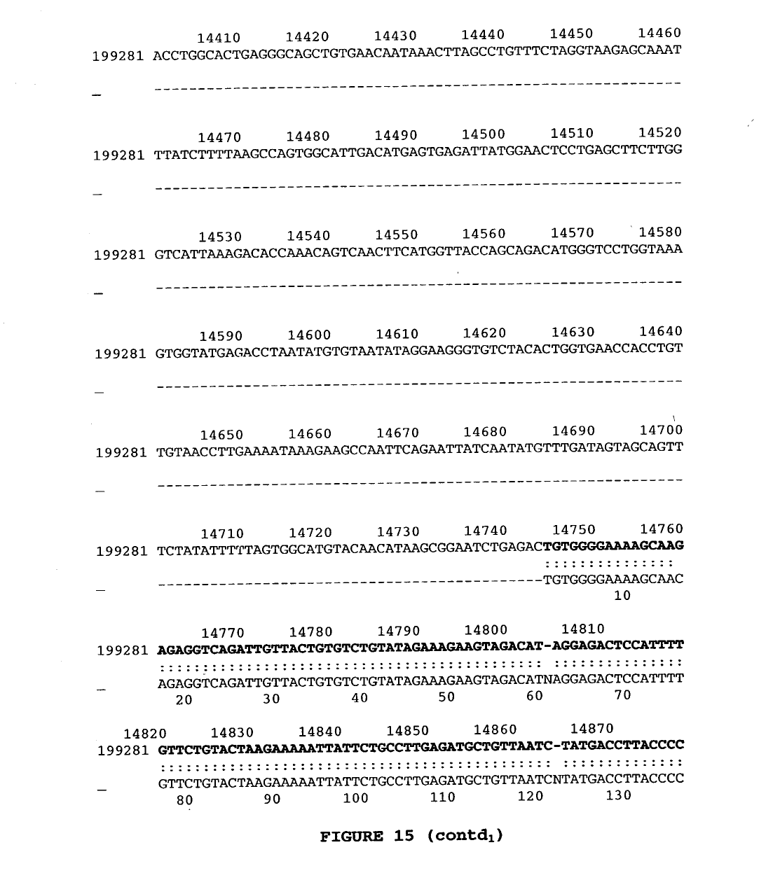

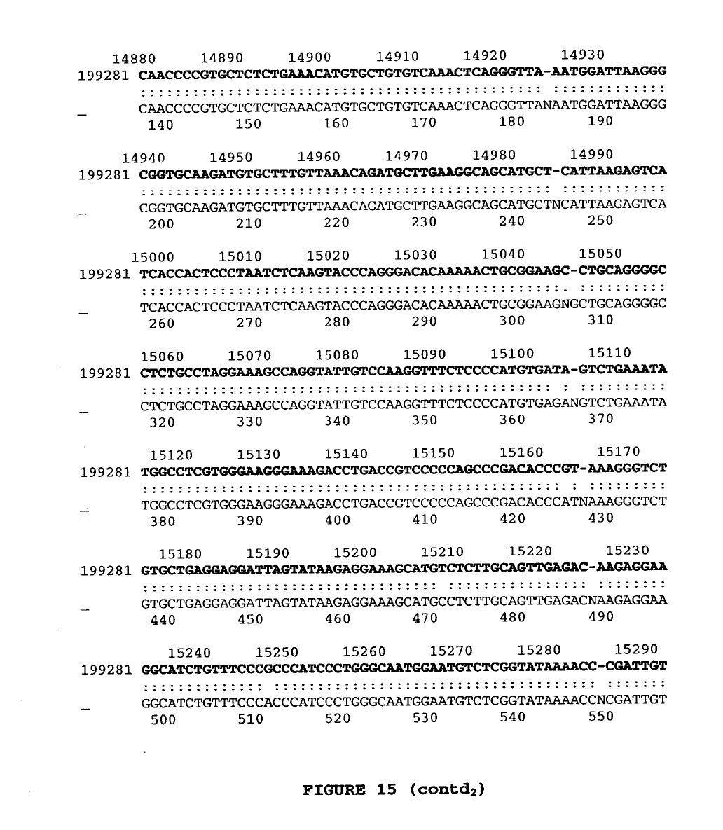

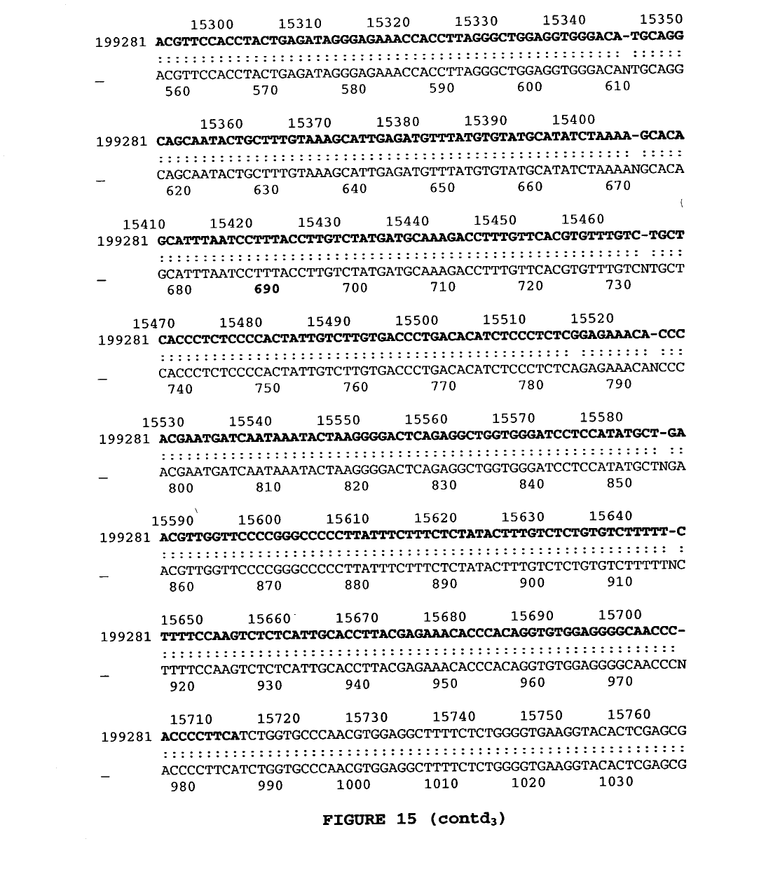

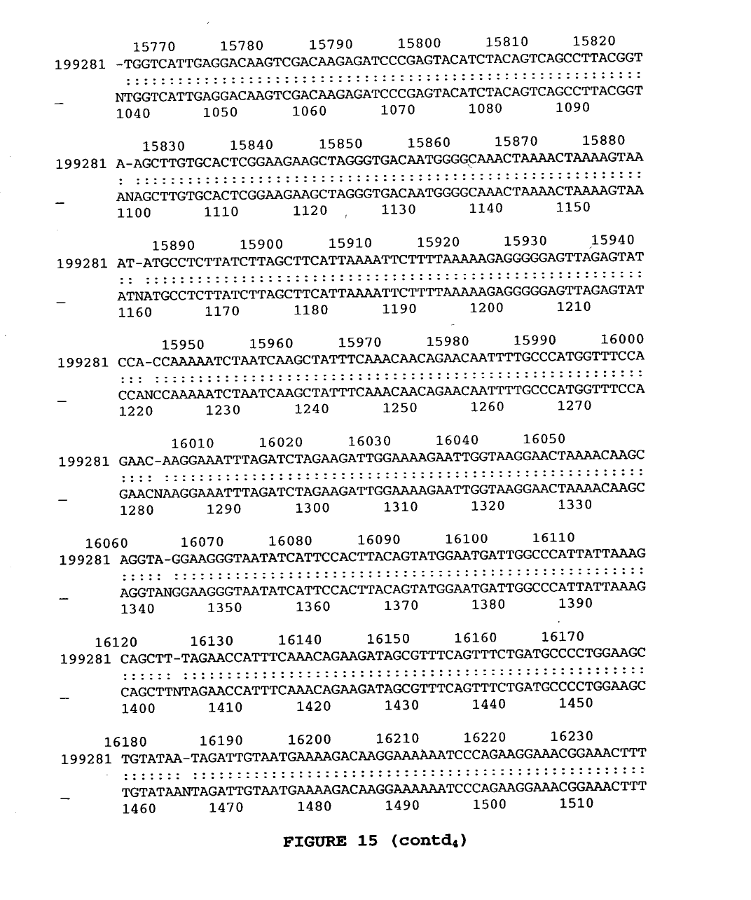

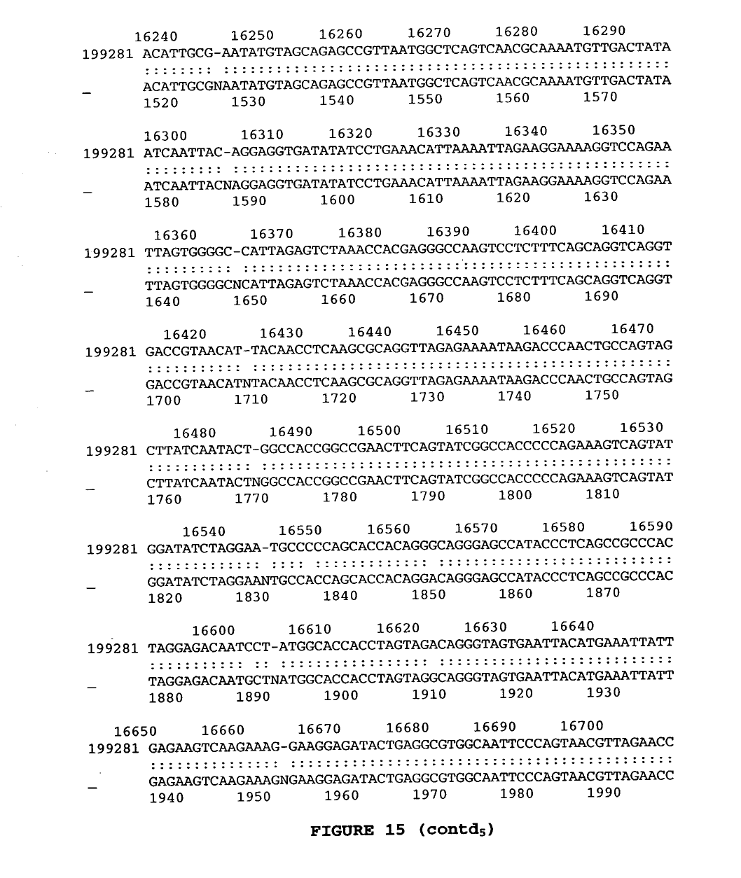

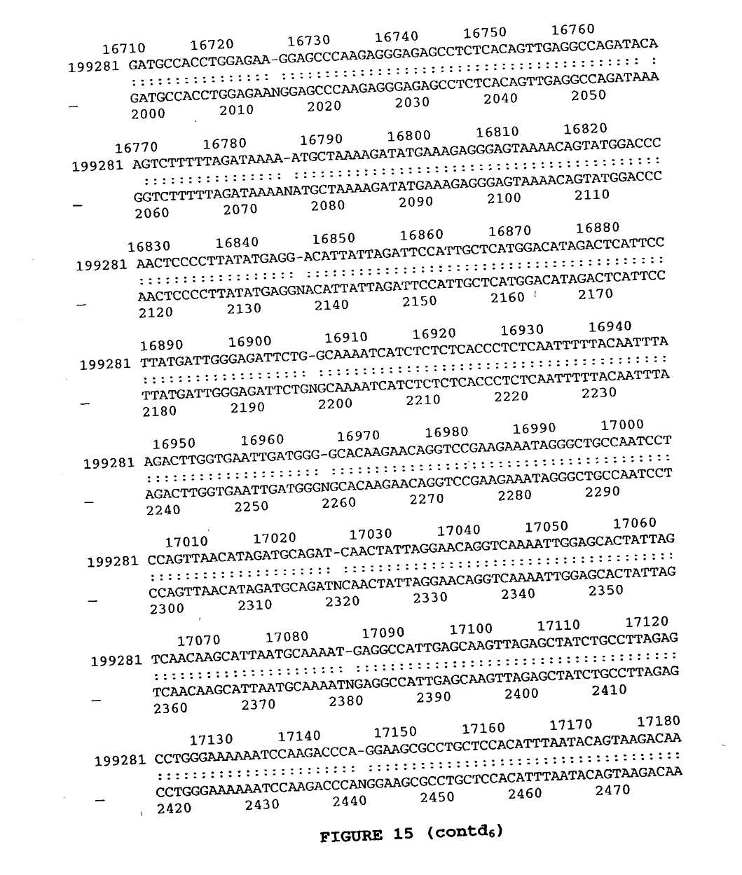

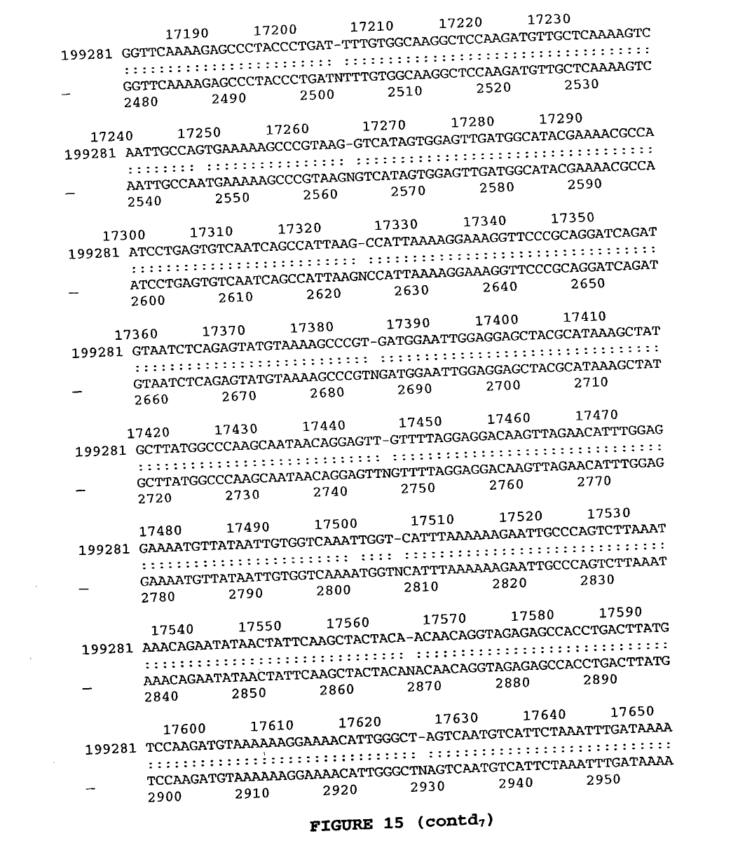

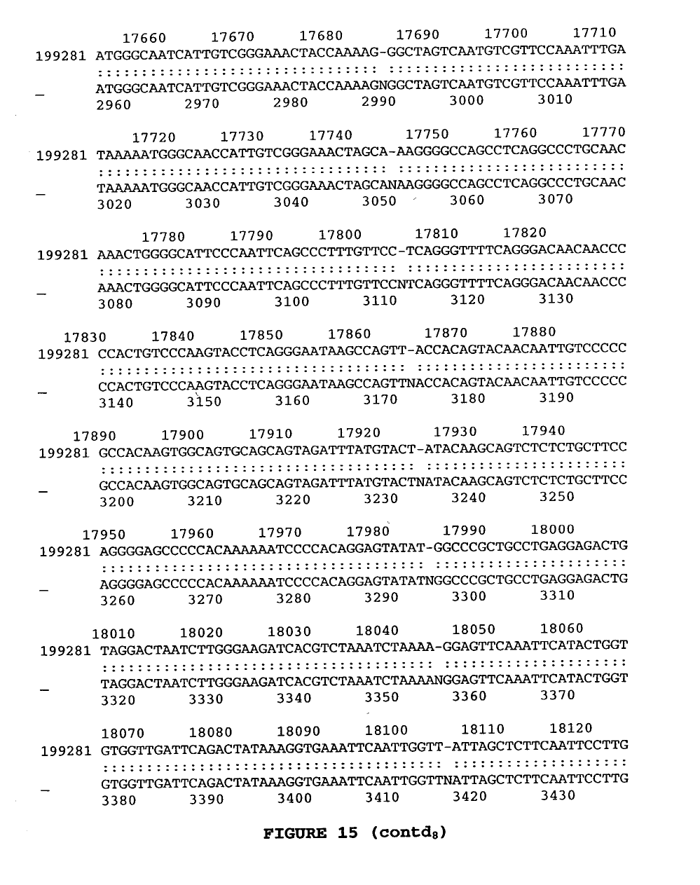

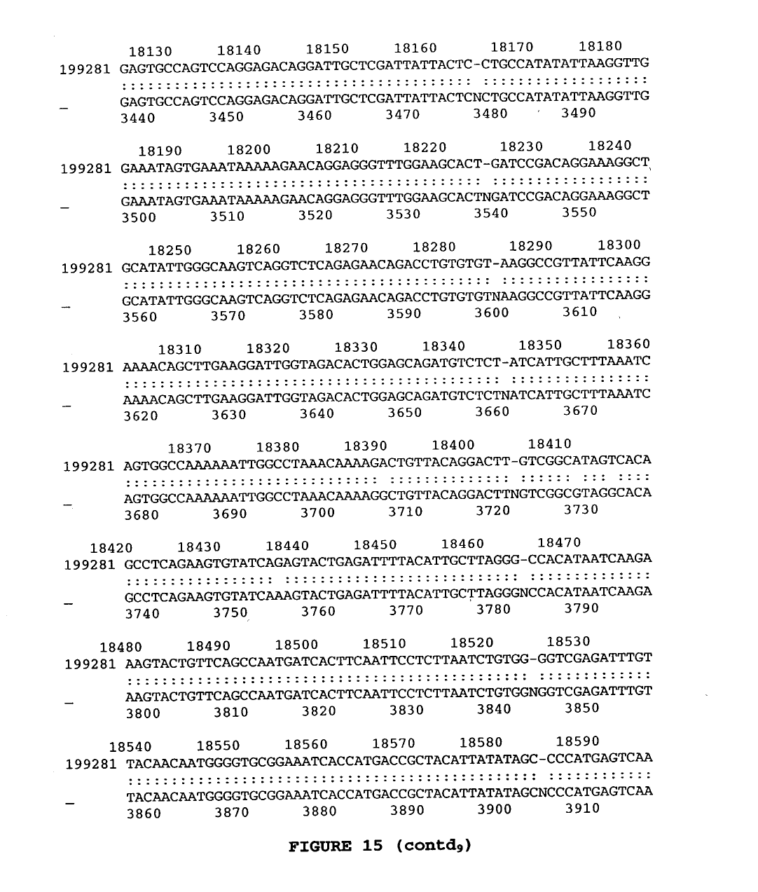

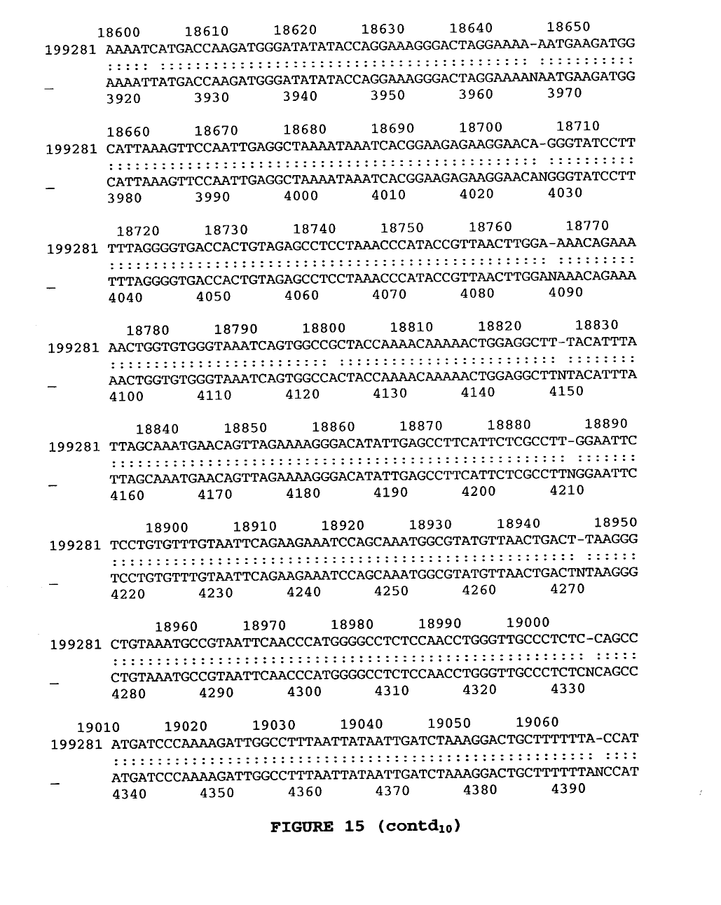

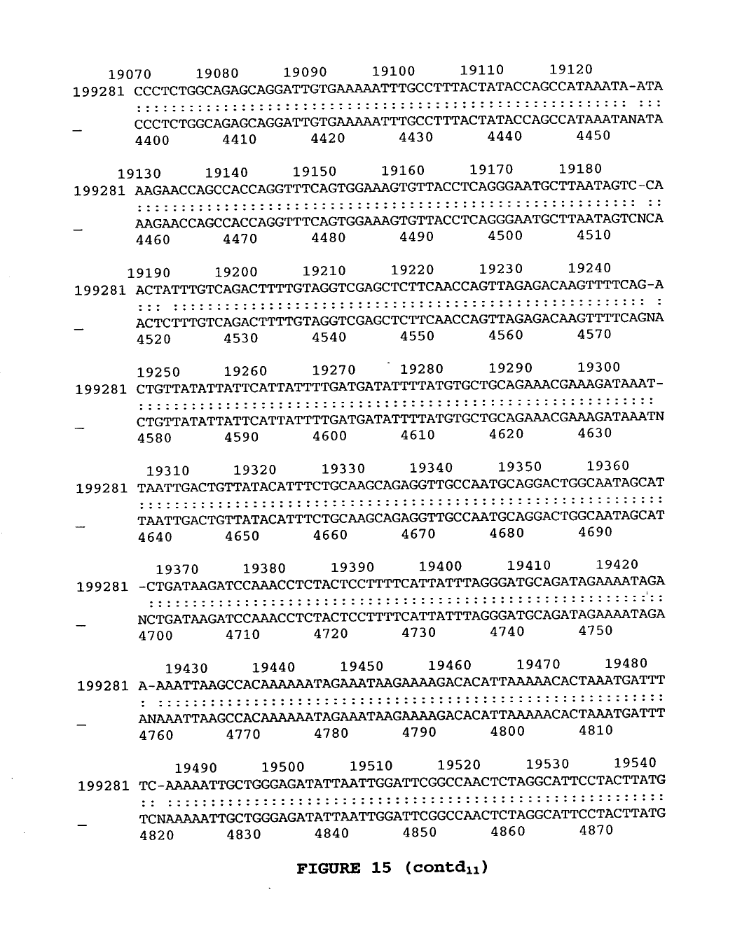

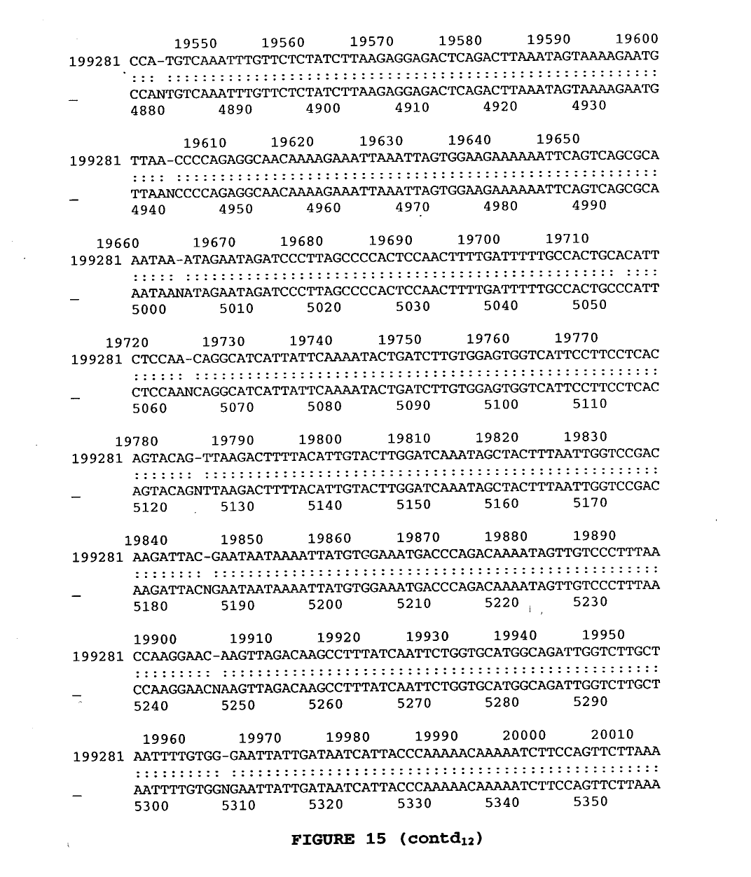

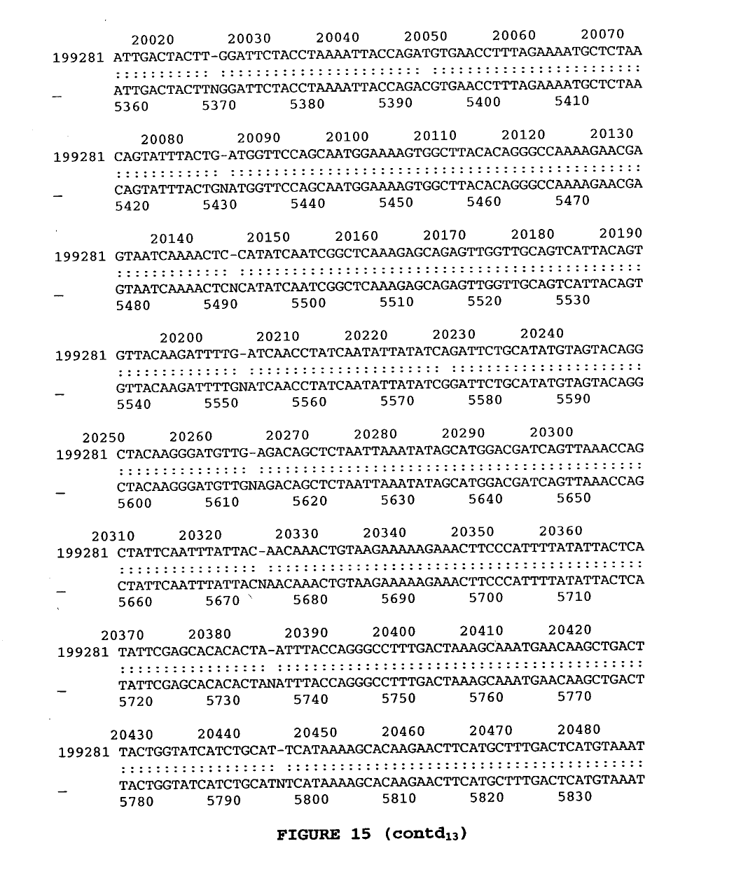

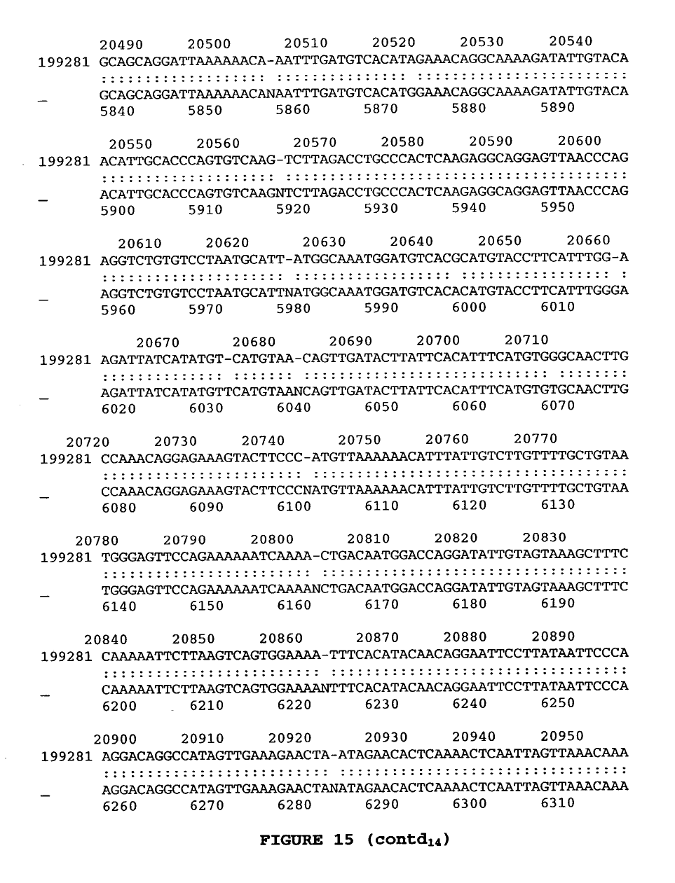

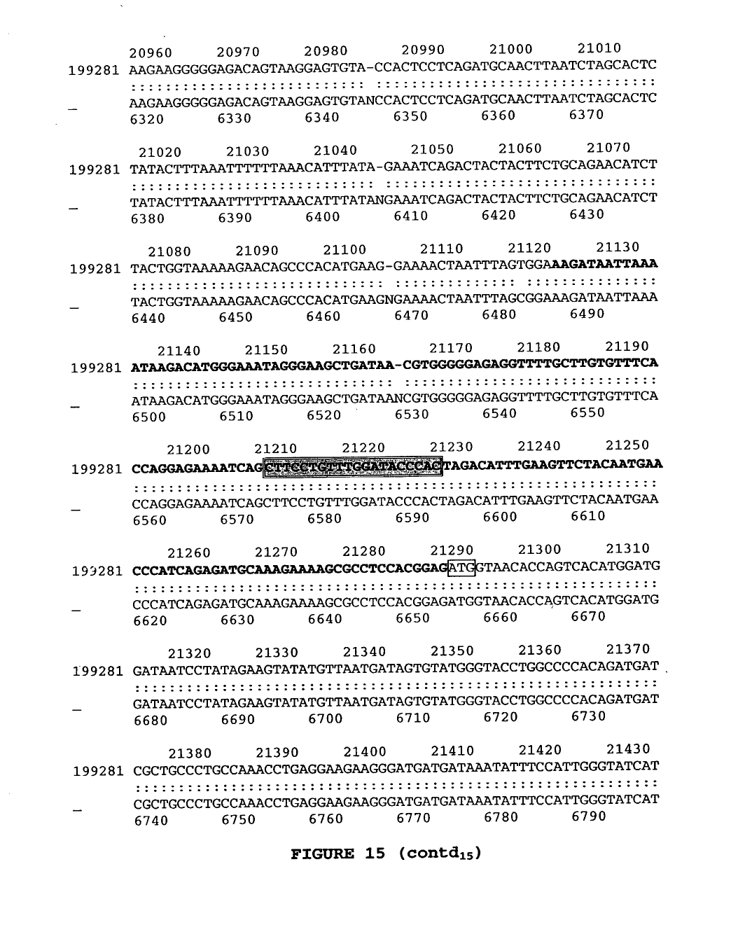

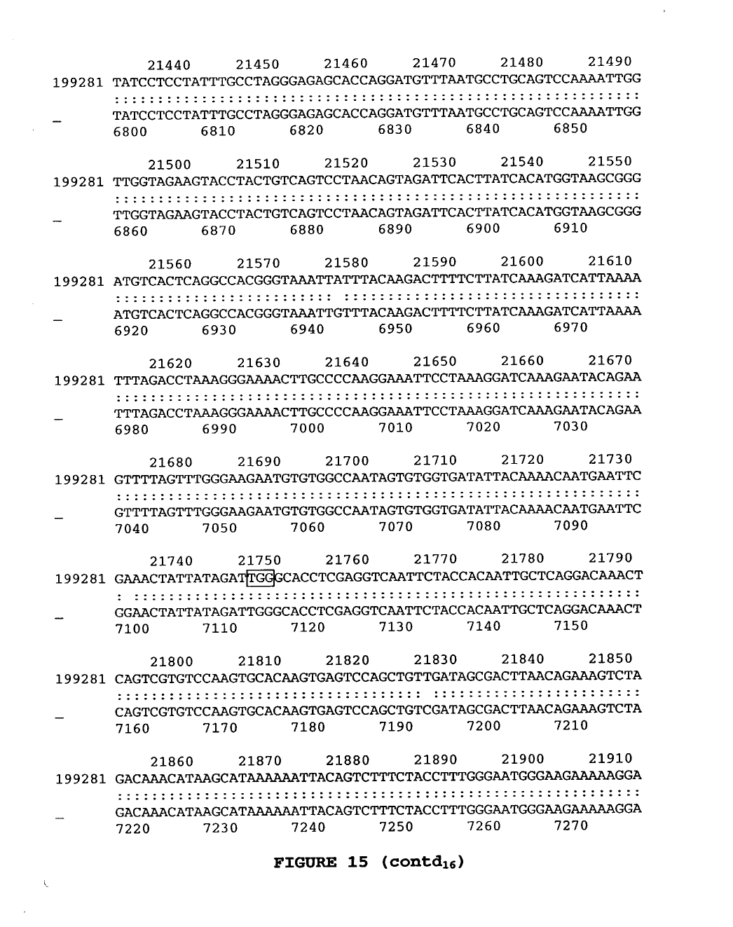

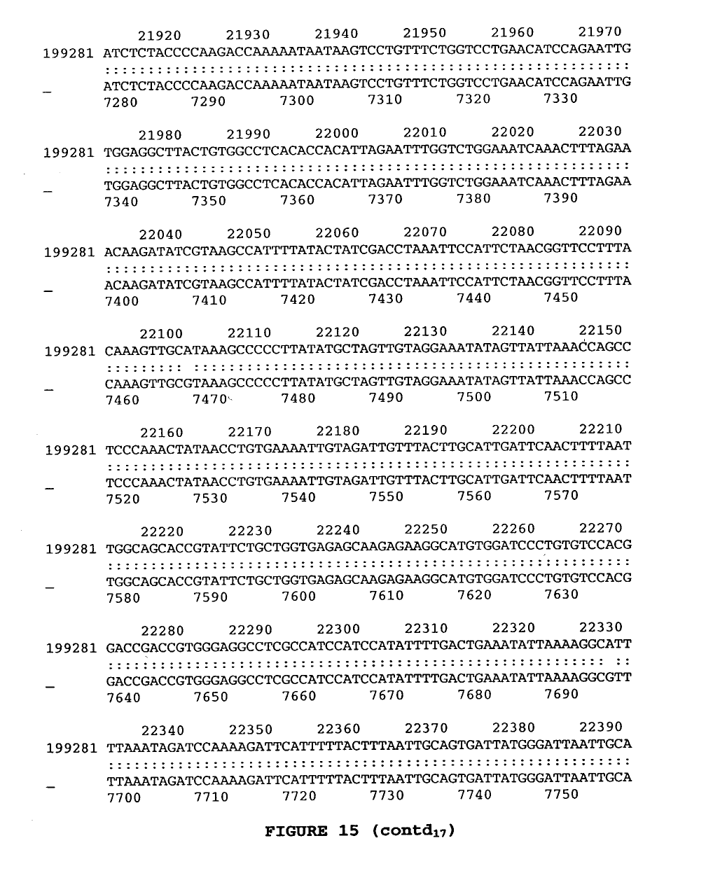

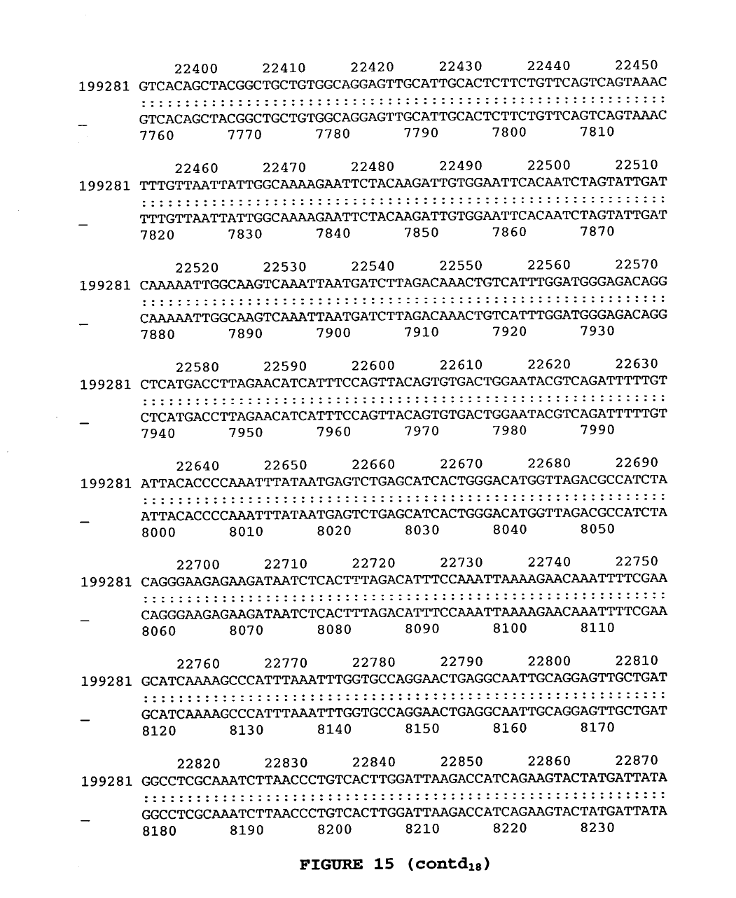

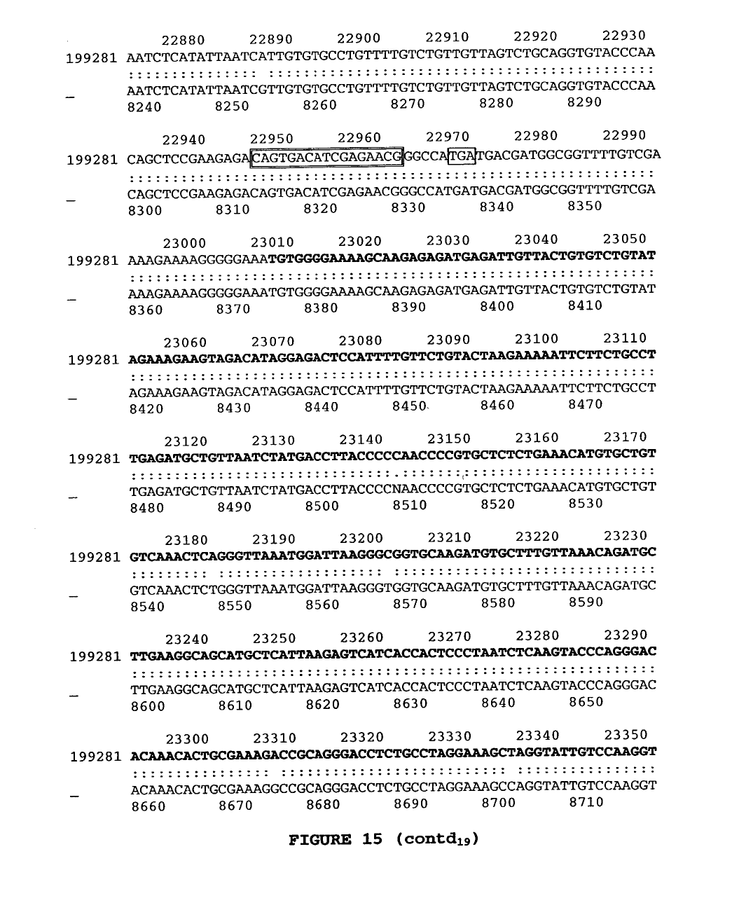

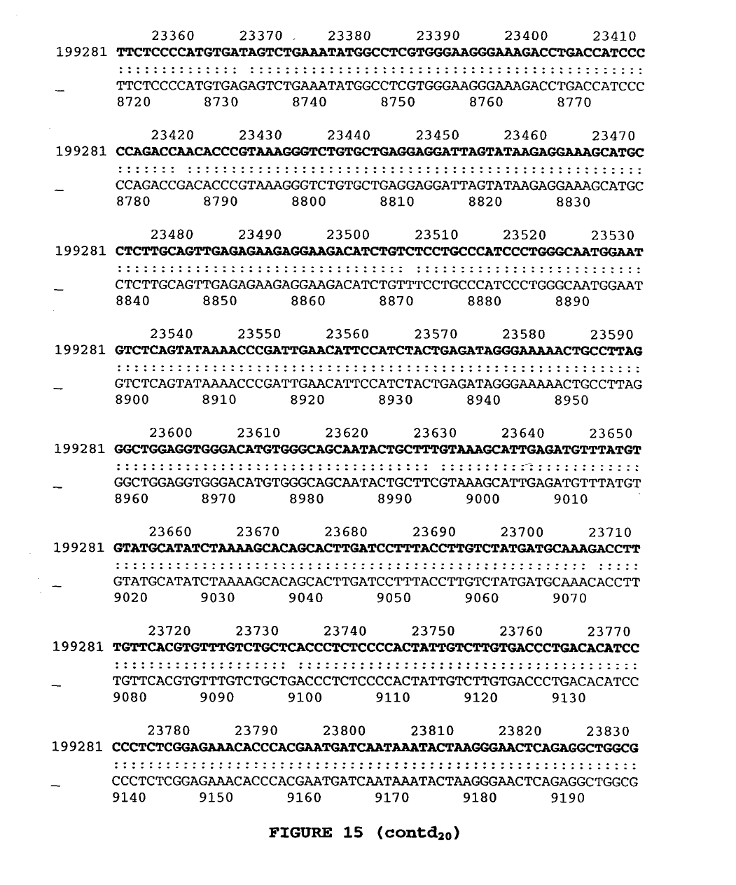

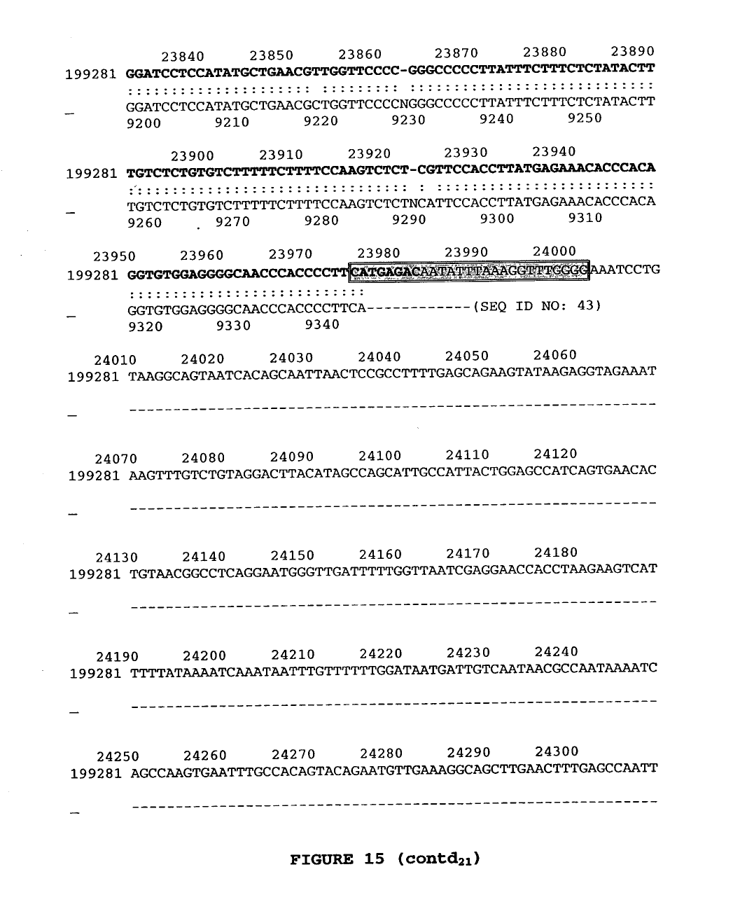

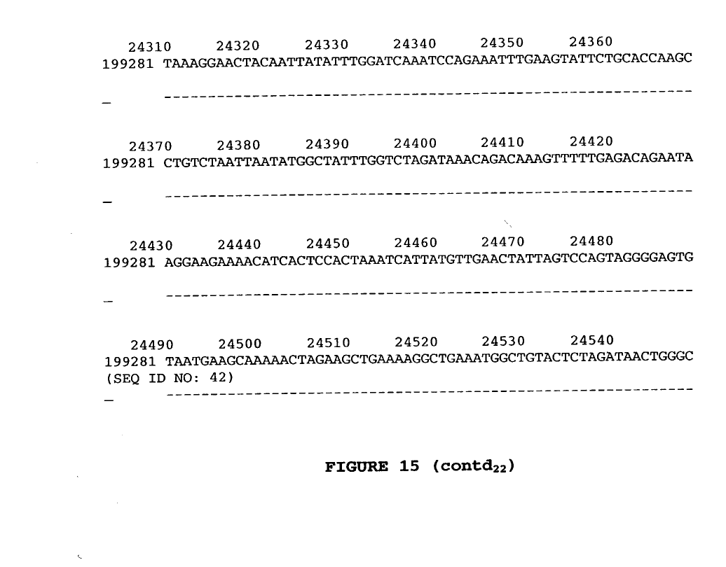

Preferred regions for use as sources of primers in the present invention are the regions within approximately 2 kb of the junction between the HERV-K18 provirus and the CD48 intron (see FIGS. 13 and 15).

As illustrated in FIG. 14, the 5′ portion of the CD48 intron constitutes a source of unique reverse primers for amplification of the HERV-K18 3′LTR or ENV. The 3′ portion of the CD48 intron (i.e. the portion which is downstream of the HERV-K18 insertion) provides a source of unique forward primers for the amplification of the HERV-K18 5′LTR. The designations “forward” and “reverse” in this context are with respect to the orientation of the HERV-K18 provirus. As the second primer, regions within HERV-K18 can be used for amplification of the LTRs or ENV.

The two primers generally correspond to genomic sequences which are less than 12 kb apart, most preferably less than 5 kb or less than 3 kb apart.

More specifically, for the amplification of ENV, the reverse primer is preferably a portion of the 5′end of the CD48 intron, flanking the HERV-K18 3′LTR, for example a portion of the CD48 intron sequence shown in FIG. 15 extending from nucleotides 23975 to 24549 of SEQ ID NO: 42) (or the corresponding region shown in inverse orientation in SEQ ID NO: 26; FIG. 13, extending from nucleotides 10001 to 11782). Any sequence having a length of between 15 to 100 nucleotides, particularly 20 to 100 nucleotides, within this region is suitable for use as a reverse primer for amplification of ENV. Particularly preferred primers are those within 200 nucleotides, especially those within 100 nucleotides, of the boundary between the HERV-K18 3′LTR and CD48 intron. A particular example is a primer comprising or consisting of the following sequence:

5′-CCCCAAACCTTTAAATATTGTCTCATG-3′

Primers K18FLR and K18FLR1 used in the Examples below are representative of such primers.

The forward primer for the amplification of ENV may correspond either to a sequence within HERV-K18 (i.e. a retroviral sequence), or it may be from the CD48 intron flanking the 5′LTR of the provirus. This latter possibility gives rise to amplification of the whole provirus when the reverse primer is in the CD48 intron flanking the 3 ′LTR of the provirus. It is preferred however that the second (forward) primer correspond to a sequence within the provirus, particularly a sequence common to all allelic variants of HERV-K18, but not present in retroviruses sharing high homology with HERV-K18, such as HERV-K10. The forward primer may or may not be unique to the HERV-K18 locus, although it is preferably unique. Particularly preferred as the forward primer for the amplification of ENV is a sequence comprising all or part of the 5′ untranslated region of HERV K18 env. This sequence is illustrated in FIGS. 5 and 15. In particular, the forward primer comprises or consists of a portion of the UTR region of ENV extending from nucleotides 21121 to 21290 as illustrated in FIG. 15. Any sequence having a length of between 15 to 150 nucleotides, or 20 to 100 nucleotides, or 30 to 100 nucleotides, within this region is suitable for use as a forward primer for amplification of ENV. Particular examples are primers comprising or consisting of either one of the following sequences:

5′-CTTCCTGTTTGGATACCCAC-3′
5′-ATCAGAGATGCAAAGAAAAGC-3′

Examples of such primers are sequences designated “FPYRO” and “K 18UTR” as used in the examples below.

For amplification of the LTR's of HERV-K18, it is again preferred to use one primer corresponding to a part of the flanking CD48 intron. More particularly, as the reverse primer for amplification of the 3′LTR, it is again preferred to use a portion of the 5′CD48 intron sequence shown in FIG. 15 extending from nucleotides 23975 to 24549. Any sequence having a length of between 15 to 150 nucleotides, particularly 20 to 100 nucleotides or 30 to 100 nucleotides, within this region is suitable for use as a reverse primer for amplification of the 3′LTR of HERV-K18, particularly sequences within 200 nucleotides, especially within 100 nucleotides, of the boundary between the HERV-K18 3′LTR and CD48 intron. Again, sequences consisting of, or comprising the following sequence (nucleotides 1762-88 of SEQ ID NO: 26):

5′-CCCCAAACCTTTAAATATTGTCTCATG-3′

are particularly preferred. Primers designated “K18FLR” and “K18FLR1” used in the Examples below are suitable examples of reverse primers for amplification of 3′LTR of HERV-K18.

The second or forward primer for amplification of the 3′LTR of HERV-K18 may correspond to a sequence within the provirus, particularly a sequence common to all allelic variants of HERV-K18, but not present in retroviruses sharing high homology with HERV-K18, such as HERV-K10. Such a sequence may be within the region spanning approximately 200 base pairs, or approximately 100 base pairs, upstream of the 3′LTR. According to this variant, the forward primer for amplification of the 3′LTR of HERV-K18 may comprise part of ENV, for example may comprise or consist of a sequence having a length of between 15 to 100 nucleotides in the region extending from nucleotides 22890 to 23010 illustrated in FIG. 15. A particularly preferred primer for amplification of the 3′LTR of HERV-K18 comprises or consists of the sequence (nucleotides 23-39 of SEQ ID NO: 39):

5′CAGTGACATCGAGAACG 3′

The sequence designated “K18LTR3”, used in the Examples below, is an example of such a primer.

Alternatively, a primer for amplification of the LTRs of HERV-K18 may comprise part of the LTR sequences themselves. Indeed, as shown below in Tables II and III, and as illustrated in FIG. 10, the polymorphism in the LTR's is spread throughout the LTR and thus only part of the LTR needs to be amplified to determine genotype. Such primers have a length of approximately 20 or 30 to 300 nucleotides, for example 30 to 100, and have a sequence common to all sequences aligned in FIG. 10, or a sequence complementary thereto, for example, a sequence identical to, or complementary to, the 3′ LTR sequences illustrated in FIG. 10 between positions 1-173, 195-278, 329 620, 651-698, 700-845 of SEQ ID NOs: 15-20. Also preferred are primers having a sequence identical to, or complementary to, the 5′ LTR sequences illustrated in FIG. 10 between positions 20-300, 305-460, 505-770 of SEQ ID NOs: 15-20.

TABLE II
Examples of HERV K18 Polymorphic sites: 3′ LTR

| | Nucleotide position | | | | | | | | |
|---|---|---|---|---|---|---|---|---|---|
| | (numbering of FIG. 10) | | | | | | | | |
| | 174 | 194 | 279 | 301 | 328 | 621 | 650 | 698 | 846 |
| 3K18.1 | A | T | A | G | C | T | T | C | C |
| 3K18.2 | T | T | G | G | C | C | C | C | C |
| 3K18.3 | A | C | A | A | T | T | C | G | T |

TABLE III
Examples of HERV K18 Polymorphic sites: 5′ LTR

| | Nucleotide position | | | | | |
|---|---|---|---|---|---|---|
| | (numbering of FIG. 10) | | | | | |
| | 16 | 301 | 464 | 485 | 503 | 771 |
| 5K18.1 | G | C | T | A | G | A |
| 5K18.2 | C | G | C | G | A | C |
| 5K18.3 | G | C | T | G | G | C |

Genotyping may also be carried out by amplification and sequencing of the 5′0LTRs. For amplification of this region, a forward primer corresponding to a part of the 3′portion of the CD48 intron is preferred. This region is shown in FIG. 15 from positions 13982 to 14744 (SEQ ID NO: 42). Any sequence having a length of between 15 to 150 nucleotides, particularly 20 to 100 nucleotides or 30 to 100 nucleotides, within this region is suitable for use as a forward primer for amplification of the 5′LTR of HERV-K18, particularly sequences within 200 nucleotides, especially within 100 nucleotides, of the boundary between the HERV-K18 5′LTR and CD48 intron.

As reverse primer for amplification of the 5′LTR of HERV-K18, a sequence within HERV-K18 is normally used. Suitable examples are sequences within the UTR of ENV as described above.

Once the chromosomal DNA has been amplified, it is analyzed for single nucleotide polymorphisms, using any of the techniques mentioned above. Direct sequencing, primer extension analysis and RFLP are particularly preferred.

After determination of the sequence of the amplified fragments, the HERV-K18 genotype of the analyzed human DNA is recorded as 1/1, 2/2, 3/3, 1/2, 1/3, 2/3, depending on the identified HERV-K18 1 (1), -18.2 (2), or -18.3 (3) allele, wherein 1/1 represents homozygous for allele K18.1, 1/2 represents heterozygous K18.1/K18.2 etc. In Caucasian populations, the genotypes appearing most frequently appear to be 2/2 and 1/1, with 3/3 being rather rare. In other ethnic groups the distribution may be different. It is also possible that other alleles exist in other populations. The theoretical frequency of the occurrence of an allele can be predicted applying the Hardy-Weinberg equilibrium. In the present case, this equilibrium predicts that the 1/1 genotype should occur at approximately twice the frequency which is actually observed. This could indicate a selective pressure against the 1/1 genotype, which may indicate a predisposition to IDDM, or to any disorders involving the HERV-K18 superantigen.

According to a preferred embodiment of the invention the genotyping of the HERV-K18 locus is carried out together with the genotyping of at least one additional locus linked to a disorder involving the HERV-K18 provirus. This provides a more effective detection method and allows a more specific detection of a particular disorder. Examples of disorders involving the HERV-K18 provirus are autoimmune disease, particularly IDDM, lupus etc. For IDDM, suitable loci which may be combined with the HERV-K18 genotyping include:

i) the TCRβV locus

ii) an HLA class II locus (IDDM1)

iii) the INS locus (IDDM2)

It is particularly preferred to combine the HERV-K18 genotyping with genotyping of two or more of these loci for a highly specific diagnosis or determination of predisposition for diabetes.

Analysis of the TCRβV locus is particularly preferred, wherein the genotyping comprises determination of the presence or absence of the Vβ7.2 and/or the Vβ13.2 gene. In fact, a 15 kb deletion polymorphism lying within the human TCRβV (TCR) locus has previously been reported [Seboun, 1989 #1205; Rowen, 1996 #1207]. The allelic nature of this polymorphism was verified in family studies, and mapping data allowed localization of one area of deletion (del) among the V gene segments genes. The gene frequency for the allelic form was 0.37/0.61, indicating that this polymorphism is widespread.

The combination of the method for identifying human TCRβV with the method for identifying HERV-K18 alleles or with other IDDM susceptibility loci such as IDDM1 and 2 described in this application represents a novel technology for identifying individuals susceptible of developing autoimmune diseases such as diabetes.

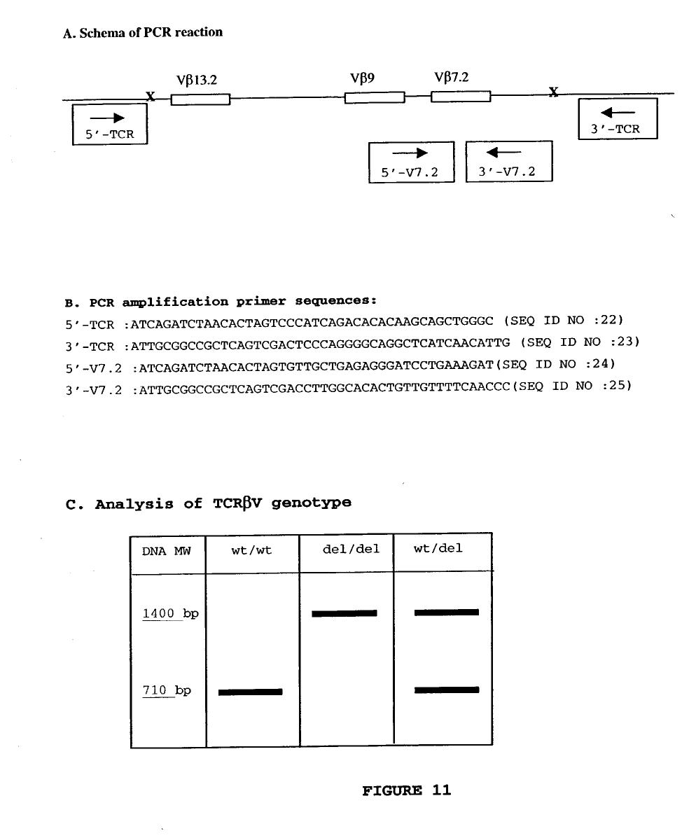

For the TCRβV genotyping, any suitable technique for detection of the deletion can be used. One technique involves amplification of the locus using a plurality of sets of primers to determine whether or not the deletion is present. A schematic representation of such an amplification method is provided in FIG. 11A. According to this embodiment, two pairs of primers are used in a duplex PCR reaction, the first (for example 5′-TCR and 3′-TCR illustrated in FIG. 11A) corresponds to sequences immediately flanking the deletion site, and amplifies the DNA only if the deletion is present (otherwise the primers are too far apart to give a positive amplification). The second pair (5′-V7.2 and 3′V7.2 illustrated in FIG. 11A) gives a positive amplification only for wild type genotypes (i.e. deletion absent), since it corresponds to a sequence within the deletion. The sequences of these loci are reported in the literature.

The genotype of the TCRβV locus is recorded as wt/wt, wt/del, del/del depending on the alleles identified.

A detailed example of this technique is provided in Example 4 below.

According to a further embodiment of the invention, the HERV-K18 genotyping is combined with genotyping at an HLA Class II locus, wherein the genotyping comprises determination of the allelic variation of at least one DR gene and/or at least one DQ gene, and/or at least one DP gene. Genotyping of this locus is well known, and methodologies therefore are reported in the literature. This aspect of the invention relates to the combined HERV-K18 genotyping with the HLA Class II locus typing.

A further example of a locus whose typing may be combined with that of HERV-K18 is the INS (IDDM2) locus. Again details for the typing of this particular locus are reported in the literature.

The invention thus provides, by combined genotyping of the HERV-K18 locus with at least one of the TCRβV, IDDM1 and IDDM2 loci, a method for identifying individuals at risk for IDDM.

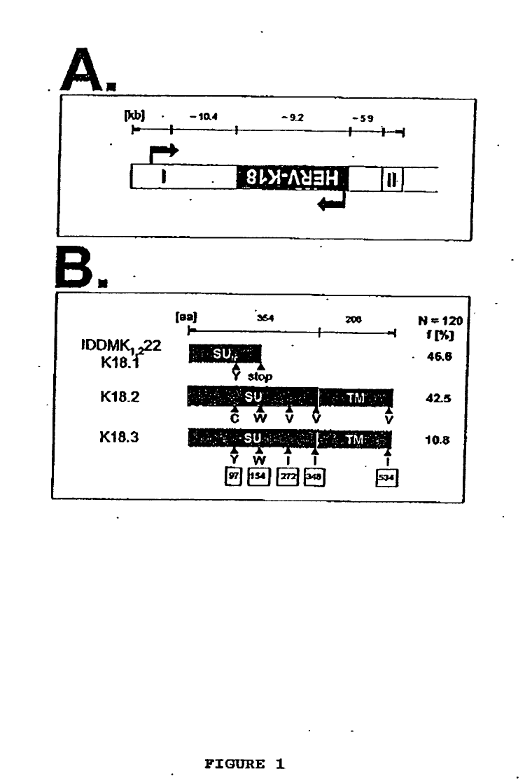

Since the HERV-K18 locus may also be associated with other disorders linked to the SAg activity of the HERV-K18 ENV, for example autoimmune diseases such as Lupus, genotyping of this locus may further provide indications relative to predisposition of individuals to those disorders. If appropriate the HERV-K18 typing can be combined with other diagnostic techniques, including genotyping, characteristic of the disorder in question to further strengthen the analysis. In the context of the present invention, IDDMK_{1.2}22 has been unambiguously assigned to the HERV-K18 locus, and it has been established that the defective HERV-K18 provirus on chromosome 1 has at least three alleles, one of which corresponds to IDDMK_{1.2}22. The integration site of the HERV-K18 provirus in the large first CD48 intron has been found to be preserved in all individuals tested. The provirus is inserted in the opposite transcriptional direction to CD48 (FIG. 1A). Allelic polymorphism has been demonstrated in the envelope gene and in the 5′ and 3′ LTRs.

The population frequency of the three HERV-K18 alleles of the envelope gene has been analyzed. The IDDMK_{1 2}22 ENV coding sequence was found in 46.6% of chromosomes and was designated allele K18.1 (FIG. 1B). Two envelope sequences similar to IDDMK_{1.2}22, but without its premature stop codon were obtained at frequencies of 42.5% (allele K18.2) and 10.80 (allele K18.3). K18.2 is identical to a published sequence (Tönjes et al., 1999). K18.3 has never previously been reported. Two additional variants K18.1′ and K18.2/3′, were found only once and based on their low frequency they may be either mutations or true alleles. These variants are described in detail in the Examples below.

The unambiguous assignment of IDDMK_{1.2}22 to HERV-K18 had not been made previously for a number of reasons. For example, the published HERV-K18 LTR sequence (Ono et al. 1986) turned out to be identical to K18.2, which is as distantly related to the IDDMK_{1.2}22/K18.1 and K18.3 LTRs as it is from the HERV-K10 LTRs (7%). This explains why the IDDMK_{1.2}22/K18.1 LTR sequence originally appeared as an independent entity, identical neither to K18 nor to K10.

IDDMK_{1.2}22 encodes superantigen (SAg) activity within the envelope gene. The present inventors have established that the truncated and full-length HERV-K18 envelope alleles all encode SAgs with identical specificity.

The present inventors have also devised techniques for analyzing the polymorphism of the HERV-K18 locus in individuals. This has in turn provided a means for assessing the predisposition to disorders linked to the HERV-K18 locus, for example, disorders associated with the expression of SAg activity.

One particularly important disease which has been linked to IDDMK_{1.2}22 is insulin-dependent diabetes mellitus (IDDM). IDDM is an autoimmune disease due to the aggression of the β islets of Langerhans cells by islet-reactive T cells. The existence of genetic control has long been known, since the disease involves a strong hereditary component. The problem is complicated by the multiplicity of predisposing genes, by the existence of protector genes, and by the relative low penetrance of predisposition. Additionally, the disease is heterogeneous with variable rapidity of progression, exemplified by the difference in age onset. There may even exist particular subsets of patients in whom pathophysiology (and consequently the genetics) are clearly different from the bulk of other patients.

The search for predisposition genes has identified HLA (IDDM1) and insulin (IDDM2) genes as the major candidates associated with IDDM onset. The potential association of IDDMK_{1.2}22/HERV-K18 with IDDM, and particularly the discovery of the existence of allelic variation within HERV-K18 provides a further avenue of investigation for determination of predisposition to the disease.

A further genetic locus which may also play a role in favoring IDDM onset is the T cell receptor (TCR) locus. Genetic polymorphisms involving a large deletion within this locus (TCRβV) have been reported.

The present invention describes a novel method for identifying genetic predisposition to type I diabetes (IDDM) by analyzing the genetic polymorphism (genotyping) at at least one of 4 different loci. Two of these loci have not yet been linked to IDDM (HERV-K18 and TCRβV), whereas two other loci have already been identified as IDDM predisposition genes (IDDM1, the HLA class II region, and IDDM2 or INS, the insulin gene region).

Genotyping of the HERV-K18 locus for IDDM genetic predisposition is novel. The HERV-K18 locus and protein products is genetically and structurally distinct from the other HERV loci of the K family, such as HERV-K10. Genotyping for the TCR deletion in relation to genetic predisposition to IDDM is also novel. In addition, it is proposed that the combined value of polymorphism at locus HERV-K18 with polymorphism at one or more of the three other loci (TCRβV, IDDM1, and IDDM2) represents a significant improvement of the genotyping methodology for IDDM predisposition.

### EXAMPLES

### Example 1

### Identification of 3 Alleles of the HERV-K18 ENV Gene

The following example describes the genomic organization, DNA and protein sequences of 3 alleles of the HERV-K18 ENV gene.

A. Genomic Organization

The HERV-K18 locus was analyzed on both chromosomes of 60 healthy individuals. The integration site of the HERV-K18 env gene (also referred to as IDDMK-18) was found within the first intron of CD48 in all individuals tested (FIG. 1A). The provirus was positioned in the opposite transcriptional direction relative to CD48.

To position the K18 envelope gene and 5′ LTR with respect to the first and second CD48 exon, PCR was performed with primers CD48E1F and K18B1F and CD4811F and CD48E2R, respectively.

Oligonucleotides:

Mapping of K18 provirus in CD48 gene:

| | | |
|---|---|---|
| CD48E1 F | 5′ CACAGATCTAGAACTAGTGCCACCATGTGCTCCAGAGGTTGG 3′ | (SEQ ID NO: 27) |
| K18BIF | 5′ CTGTCATTTGGATGGGAGACAGGC 3′ | (SEQ ID NO: 28) |
| CD48I1F | 5′ CACGGATCCCAGATTCCGCTTATGTTGTACATGC 3′ | (SEQ ID NO: 29) |
| CD48E2R | 5′ CACGTCGACGGAGACCACGGTTCATATGTACCAAGTGAC 3′ | (SEQ ID NO: 30) |

(Primer designations E1 = Exon 1; E2 = Exon 2; I1 = Intron 1, F = forward; R = reverse, BI = BamHI)

Amplification of CD48 Gene:

| | |
|---|---|
| 5′CACAGATCTAGAACTAGTGCCACCATGTGCTCCAGAGGTTGG3′ | (SEQ ID NO: 31) |
| 5′CACGCGGCCGCAGAGTCGACTCAATCAA TCAGGTAAGTAACAGC 3′ | (SEQ ID NO: 32) |

B. Polymorphism in the ENV Region of HERV-K18

a) Analysis Using Standard “Sanger” Reaction:

The env-LTR fragments of K18 proviruses from 60 different individuals were amplified by PCR with primers K18UTR and K18FLR. Primer K18UTR corresponds to part of the unique 5′untranslated region of HERV K18 ENV (see SEQ ID NO: 6, FIG. 5; and SEQ ID NO: 42, FIG. 15). Primer K18FLR corresponds to the region flanking HERV-K18 in the CD48 intron 1 (5′ end, adjacent to the HERV-K18 3′LTR: see FIGS. 14 and 15). These primers allow amplification of the whole ENV region of the provirus.

PCR Amplification Primers:

| | |
|---|---|
| K18UTR: 5′-ATCAGATCTAACACTAGTAACCCATCAGAGATGCAAAGAAAAGC-3′ | (SEQ ID NO: 33) |
| K18FLR: 5′-ATTGCGGCCGCTCAGTCGACCCCAAACCTTTAAATATTGTCTCATG-3′ | (SEQ ID NO: 34) |

PCR products were i) directly sequenced using the standard “Sanger” reaction; ii) subcloned and the presence of all polymorphic sites was confirmed on single molecular clones by sequencing (GenBank accession number AF012336). HERV-K18 sequencing primers:

| | | |
|---|---|---|
| Seq.prim.pos97: | 5′-ATCAGATCTAACACTAGTTGCCACACTGGTAACACCAGTCACATGG-3′ | (SEQ ID NO: 35) |
| Seq.prim.pos154: | 5′-AGAATGTGTGGCCAATAGTGT-3′ | (SEQ ID NO: 36) |
| Seq.prim.pos272: | 5′-ATGGATGGCGAGGCCTCCCAC-3′ | (SEQ ID NO: 37) |
| Seq.prim.pos348: | 5′-AGAGAAGGCATGTGGATCCCT-3′ | (SEQ ID NO: 38) |

DNA sequencing identified single nucleotide polymorphisms (SNPs) that can be grouped into 3 distinct alleles. These alleles are identified as HERV-K18.1, -K18.2 and -K18.3 and appear at a frequency of 46.6%, 42.5%, and 10.8% in the normal human population, respectively (FIG. 1B).

Two additional variants were found only once and based on their low frequency they may be either mutations or true alleles. The first variant, candidate allele K18.1′ (SEQ ID NO: 12), had an envelope sequence identical to K18.1 but a divergent 3′ LTR. The second variant, candidate allele K18.2/3′, had an envelope sequence intermediate between K18.2 and K18.3 (Y at position 97; W at position 154; V in positions 272 and 348; I at position 534 of SEQ ID NO: 10).

b) Analysis using Pyrosequencing:

In a manner similar to that disclosed above in Example 1.B(a), the env-LTR fragments of K18 proviruses from a further, different group of healthy individuals were analyzed on both chromosomes, using PCR amplification; This time however, PCR products were directly sequenced by pyrosequencing, a technique which enables high throughput analysis. The amplification primers used were primers FPYRO and K18FLR1. Primer FPYRO again corresponds to part of the unique 5′untranslated region of HERV K18 ENV approximately 80 nucleotides upstream of the ENV START codon (see FIG. 15). Primer K18FLR1 (SEQ ID NO: 41) corresponds to the region flanking HERV-K18 in the CD48 intron 1 (5′ end, adjacent to the HERV-K18 3′LTR: see FIGS. 14 and 15). These primers allow specific amplification of the whole ENV region of the provirus.

PCR Amplification Primers:

| | | |
|---|---|---|
| Forward primer FPYRO: | 5′-ctt cct gtt tgg ata ccc ac-3′ | (SEQ ID NO: 40) |
| Reverse primer K18FLR1: | 5′-ccc caa ace ttt aaa tat tgt ctc atg-3′ | (SEQ ID NO: 44) |

PCR products were directly sequenced by pyrosequencing at positions 97 and 154.

HERV-K18 Sequencing Primers:

For pyrosequencing, the primers were as follows:

| | |
|---|---|
| Pyroseq.pos97: | |
| 5′-ctt tga taa gaa aag tct tg-3′ | (SEQ ID NO: 45) |
| Pyroseq.pos154: | |
| 5′-tga cct cga ggt gcc-3′ | (SEQ ID NO: 46) |

Analysis of this second group of individuals using PCR followed by pyrosequencing confirmed the existence of single nucleotide polymorphisms (SNPs) identified as HERV-K18.1, -K18.2 and -K18.3. The results of this second analysis also confirm that in the normal human population, the alleles appear at a frequency of approximately 47% (HERV-K18.1), 43% (K18.2), and 10% (K18.3), respectively.

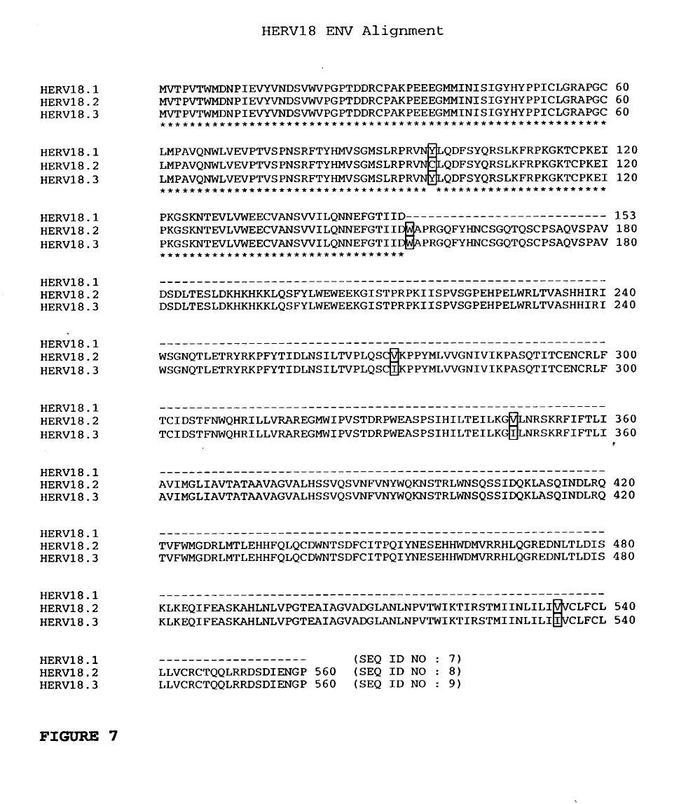

The nucleotide sequence alignment of the 3 HERV-K18 ENV alleles is represented in FIG. 8. The protein sequence alignment is represented in FIG. 7.

C. Polymorphism in the LTR Region of HERV-K18

Using PCR and restriction analysis, polymorphism was also found in the 5′ and 3′ LTR regions of HERV-K18 provirus.

A 1096 bp fragment containing the 3′K18 LTR was amplified with primers K18LTR3 and K18FLR. The product was digested with BstNI and NsiI and analyzed on 8% PAGE, which allowed to discriminate between all K18 genotypes.

Amplification of 3′K18 LTR for Typing:

| | | |
|---|---|---|
| K18LTR3 | 5′GACAGATCTCACACTAGTGCTACAGTGACATCGAGAACG 3′ | (SEQ ID NO: 39) |
| K18FLR | 5′ATTGCGGCCGCTCAGTCGACCCCAAACCTTTAAATATTGTCTCATG3′ | (SEQ ID NO: 5) |

The sequences of the different 5′ and 3′ LTR's are shown in FIG. 9. The sequences are aligned in FIG. 10. Tables II and III above show the positions characteristic of the different alleles. Genotyping can be carried out on the basis of either the 5′ or the 3′ LTR.

### Example 2

### Superantigen Activity of the HERV-K18 ENV Gene Products

A preferential expansion of T cells expressing the Vβ7 T cell receptor was found in early diabetic patients, linking Vβ7 T cell expansion to diabetes onset [Conrad, 1994 #1220]. Previous published results have demonstrated that the HERV-K18 gene product specifically stimulates a subset of T cells expressing the Vβ7 T cell receptor [Conrad, 1997 #1218]. Here, we demonstrate that this stimulatory activity (=superantigen activity) is observed with the gene products of all 3 HERV-K18 alleles identified and described in this application (FIG. 4).

We show that the HERV-K-18 gene products, in addition to stimulating Vβ7 T cells, also stimulated T cells expressing the Vβ13.1 T cell receptor. This activation of both Vβ7 T cells and Vβ13.1 T cells by HERV-K18 gene products is relevant, since both Vβ7 and Vβ13.1 T cell expansion was observed in lymphocytes of early IDDM patients [Luppi, 2000 #1223].

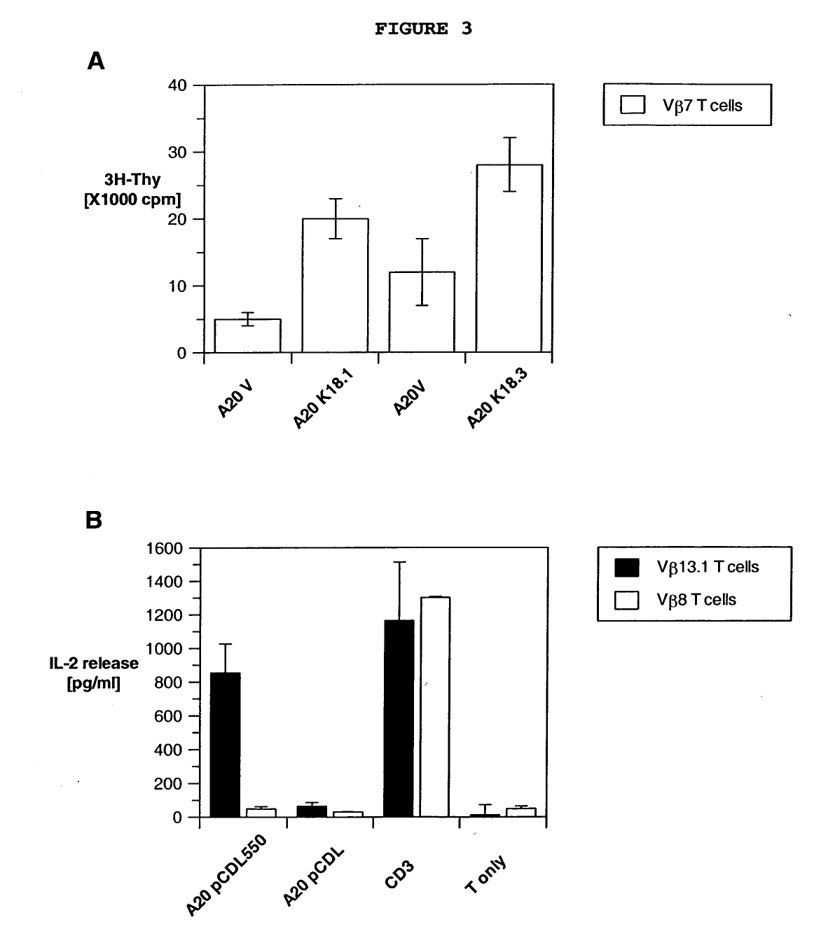

The 3 HERV-K18 alleles display superantigen activity and specifically stimulate T cells expressing the Vβ7 and Vβ13.1 T cell receptors (FIG. 3). A20 cells expressing HERV-K18.1 and -K18.3 specifically stimulated proliferation of T cells expressing the Vβ7 T cell receptor (FIG. 3A).

CD4⁺ Vβ7 T cells were derived from a SAg responsive donor by repeated cycles of stimulation with Vβ7 antibody 3G5 (Coulter) and syngeneic feeders. 1-5×10⁵ A20 transfectants were incubated with 10⁵ Vβ7 T cells and 10⁵ irradiated syngeneic PBL as feeders in 96 well plates. After 48h, ³H-Thymidine was added for 18h and incorporation measured.

For this SAg assay, transfectants expressing ENV proteins were generated as follows. Bicistronic expression cassettes containing enhanced yellow or green fluorescent protein (EYFP/EGFP) as reporters were generated. Cells were split 24h before electroporation, 10×10⁶ cells were resuspended in 250 ml RPMI with 10 μl (1 μg/μl) linearized plasmid in TE pH 8.0, in the presence of 1 μl (1 μg/μl) linearized blasticidin resistance gene (BSD, Invitrogen). Stable integrants were selected for resistance of 10 μg/ml BSD. Bulk transfectants were FACS sorted for EYFP/EGFP fluorescence, cloned by limiting dilution and maintained for no longer than 30 days in continuous culture at 5 μg/ml BSD. Single clones exhibiting mean fluorescence intensities (MFI) of EYFP/EGFP fluorescence in the range of >5 and <10 were selected and this was critical for SAg function. The bicistronic cassette with EGFP allowed to select for the lowest functional SAg expression levels and was superior to the EYFP reporter.

In addition, A20 cells expressing HERV-K18.1 specifically stimulated IL-2 release from T cells expressing the Vβ13.1 T cell receptor, but not T cells expressing the Vβ8 T cell receptor (FIG. 3B).

### Example 3

### Method for Identifying HERV K-18 Alleles (18.1, 18.2, and 18.3)

The following method describes a technique for identifying HERV-K18 alleles starting from human DNA. The method involves 3 steps: A) PCR amplification of human DNA, B) analysis of single nucleotide polymorphisms, and C) recording of the genotype corresponding to the HERV-K18 alleles.

A. PCR Amplification

The PCR and sequencing primers for amplifying full-length K-18 ENV genes from human DNA are described in Example 1. For amplification of the ENV region, Primers K18UTR and K18FLR described in Example 1 can be used as PCR amplification primers, inter alia. For amplification of the 3′LTR region, primers K18LTR3 and K18FLR can be used, inter alia.

B. Analysis of Single Nucleotide Polymorphisms

The PCR products are used as starting material for identifying single nucleotide polymorphisms (SNPs) distinguishing the HERV-K-18 alleles. The sequencing primers (seq.prim.) for identification of SNPs are presented in Example 1 above.

C. Recording the HERV-K18 Genotype.

The HERV-K18 genotype of human DNA samples is recorded according to the corresponding alleles identified by sequencing. Thus, the genotype is recorded as 1/1, 2/2, 3/3, 1/2, 1/3, 2/3, depending on the identified HERV-K18 1 (1), -18.2 (2), or -18.3 (3) allele.

### Example 4

### A Method for Identifying 2 TCRβV Alleles (wt and del)

The following example describes a method for identifying a deletion polymorphism lying within the human T cell receptor (TCR) locus. The presence of additional TCRβV genes or, alternatively, the absence of certain TCRβV genes may have an impact upon immune responses and susceptibility to autoimmune diseases such as diabetes.

The method for identifying TCRβV alleles given here as an example involves 2 steps: 1) PCR amplification of the TCRβV locus, and 2) analysis of the TCRβV genotype.

A. PCR Amplification

A method for identifying deletion polymorphism in the TCRβV gene complex has previously been identified [Boysen, 1996 #1210]. In this application, we claim the combination of the genotyping for HERV-K18 with the genotyping for the TCR alleles. For this, a PCR technique is used for identifying the 2 TCR Vβ alleles from human DNA (FIG. 11). Both the wild-type (wt) and deletion (del) alleles are located within the T cell receptor locus of chromosome 7. Two parallel PCR reactions are performed to distinguish between the 2 alleles. Two distinct sets of PCR amplification primers (TCR and V7) are used in each of the PCR reactions (SEQ ID NOs: 22-25; FIG. 11B).

B. Analysis of TCRβV Polymorphisms

The wild-type (wt) and deletion (del) alleles are distinguished by gel electrophoresis of the PCR products (FIG. 11C). In the case of a wt allele, a PCR product of 710 bp is identified using the 5′-V7.2 and 3′-V7.2 set of primers, whereas no PCR product is detected using the 5′-TCR and 3′-TCR set of primers. In the case of a del allele, a PCR product of 1400 bp is identified using the 5′-TCR and 3′-TCR set of primers, whereas no PCR product is detected using the 5′-TCR and 3′-TCR set of primers. The PCR fragment size is dependent on the choice of primers. The genotype of the TCR locus is recorded as wt/wt, del/del, and wt/del depending on the alleles identified by gel electrophoresis.

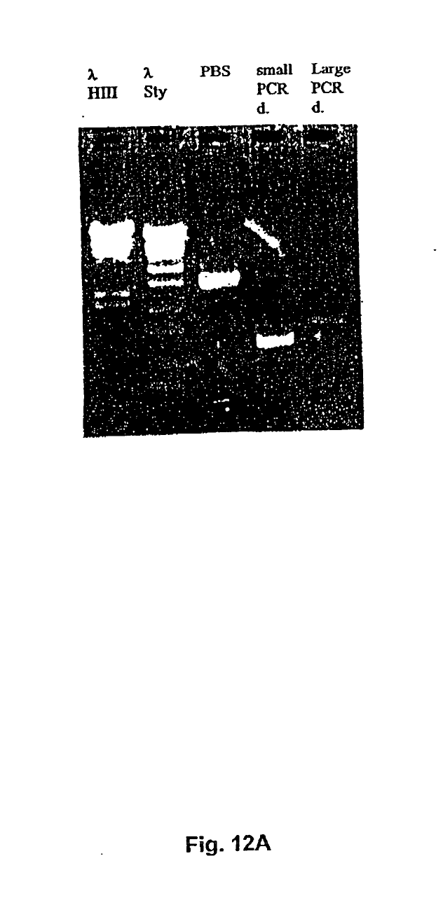

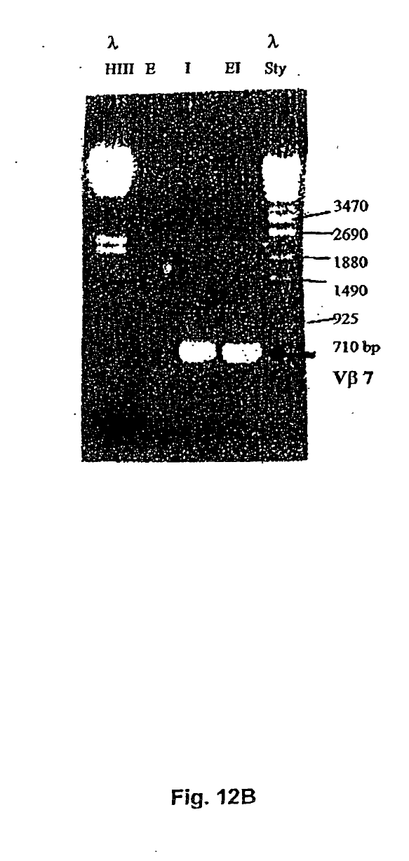

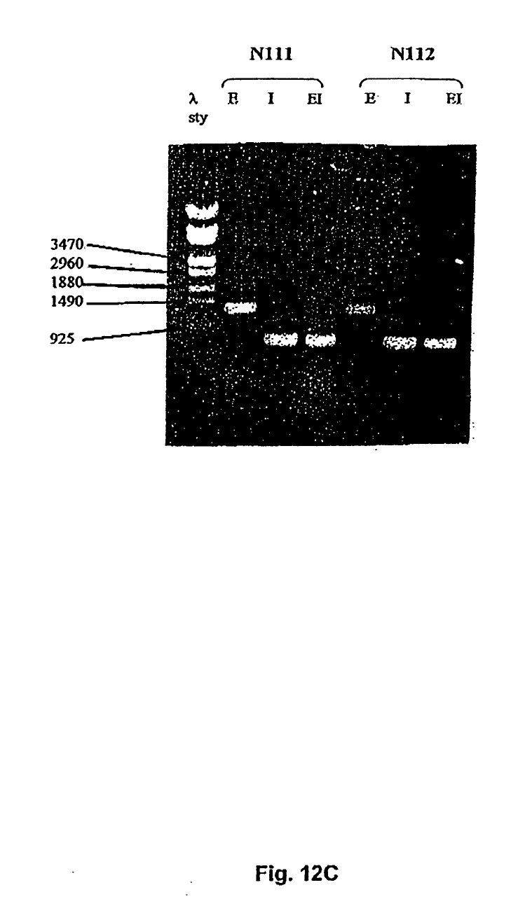

Genotyping of the two alleles (wt and del) is performed by duplex PCR on human DNA samples using the V7 and TCR primer sets (FIGS. 12A to C).

### Example 5

### A Methodology for Genotyping the Combined Loci of HERV-K18, TCRβV IDDM1, and IDDM2

The existence of genetic control of diabetes has long been known, since the disease involves a strong hereditary component [for review, see Caillat-Zucman, 2000 #1216]. The search for predisposition genes has identified the 2 major candidate set of genes, which are the HLA genes (IDDM1) and insulin (IDDM2). The methods for identifying the IDDM1 and IDDM2 have been described [Bell, 1984 #1227; Spielman, 1993 #1226; Concannon, 1998 #1224; Mein, 1998 #1225].

The combination of the method for identifying human IDDM1 and IDDM2, susceptibility genes with the method for identifying HERV-K18 and TCVβV genotypes described in this application represents a novel technology for identifying individuals susceptible of developing autoimmune diseases such as diabetes.

### REFERENCES

1. Caillat-Zucman, S., and J. F. Bach. 2000. Genetic predisposition to IDDM. Clin Rev Allergy Immunol 19:227.

2. Conrad, B., E. Weidmann, G. Trucco, W. A. Rudert, R. Behboo, C. Ricordi, H. Rodriquez-Rilo, D. Finegold, and M. Trucco. 1994. Evidence for superantigen involvement in insulin-dependent diabetes mellitus aetiology. Nature 371:351.

3. Conrad, B., R. N. Weissmahr, J. Boni, R. Arcari, J. Schupbach, and B. Mach. 1997. A human endogenous retroviral superantigen as candidate autoimmune gene in type I diabetes. Cell 90:303.

4. Luppi, P., M. M. Zanone, H. Hyoty, W. A. Rudert, C. Haluszczak, A. M. Alexander, S. Bertera, D. Becker, and M. Trucco. 2000. Restricted TCR V beta gene expression and enterovirus infection in type I diabetes: a pilot study. Diabetologia 43:1484.

5. Seboun, E., M. A. Robinson, T. J. Kindt, and S. L. Hauser. 1989. Insertion/deletion-related polymorphisms in the human T cell receptor beta gene complex. J Exp Med 170:1263.

6. Rowen, L., B. F. Koop, and L. Hood. 1996. The complete 685-kilobase DNA sequence of the human beta T cell receptor locus. Science 272:1755.

7. Boysen, C., C. Carlson, E. Hood, L. Hood, and D. A. Nickerson. 1996. Identifying DNA polymorphisms in human TCRA/D variable genes by direct sequencing of PCR products. Immunogenetics 44:121.

8. Bell, G. I., S. Horita, and J. H. Karam. 1984. A polymorphic locus near the human insulin gene is associated with insulin-dependent diabetes mellitus. Diabetes 33:176.

9. Spielman, R. S., R. E. McGinnis, and W. J. Ewens. 1993. Transmission test for linkage disequilibrium: the insulin gene region and insulin-dependent diabetes mellitus (IDDM). Am J Hum Genet 52:506.

10. Concannon, P., K. J. Gogolin-Ewens, D. A. Hinds, B. Wapelhorst, V. A. Morrison, B. Stirling, M. Mitra, J. Farmer, S. R. Williams, N.J. Cox, G. I. Bell, N. Risch, and R. S. Spielman. 1998. A second-generation screen of the human genome for susceptibility to insulin-dependent diabetes mellitus. Nat Genet 19:292.

11. Mein, C. A., L. Esposito, M. G. Dunn, G. C. Johnson, A. E. Timms, J. V. Goy, A. N. Smith, L. Sebag-Montefiore, M. E. Merriman, A. J. Wilson, L. E. Pritchard, F. Cucca, A. H. Barnett, S. C. Bain, and J. A. Todd. 1998. A search for type 1 diabetes susceptibility genes in families from the United Kingdom. Nat Genet 19:297.

Ono M, J.Virol.1986, 58, (3), 937-44.

Tönjes R., et al., J.Virol. 1999, 73 (11), 9187-9195.

Hasuike S., et al. J.Human Genet 1999, 44, 343-347.

### Equivalents

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A protein which is 98.5% to 99.9% identical to the amino acid sequence of SEQ ID NO: 8.

2. The protein of claim 1, wherein said protein has SAg activity.

3. A protein having from 1 to 5 amino acid substitutions, deletions and/or insertions with respect to the amino acid sequence of SEQ ID NO: 8.

4. The protein of claim 3, wherein said protein has SAg activity.

5. The protein of claim 1, wherein at least one of said amino acid substitutions, deletions and/or insertions occurs at one or more of positions 97, 154, 272, 348, and 534.

6. The protein of claim 5, wherein said protein has SAg activity.

7. The protein of claim 1, which is 99.0% to 99.9% identical to the amino acid sequence of SEQ ID NO: 8 over a length of 560 amino acids.

8. The protein of claim 7, wherein said protein has SAg activity.

9. A protein which is 98.5% to 99.9% identical to the amino acid sequence of SEQ ID NO: 9.

10. The protein of claim 9, wherein said protein has SAg activity.

11. A protein having from 1 to 5 amino acid substitutions, deletions and/or insertions with respect to the amino acid sequence of SEQ ID NO: 9.

12. The protein of claim 11, wherein said protein has SAg activity.

13. A protein which is 98.5% to 99.9% identical to the amino acid sequence to the amino acid sequence of SEQ ID NO: 10.

14. The protein of claim 13, wherein said protein has SAg activity.

15. A protein having from 1 to 5 amino acid substitutions, deletions and/or insertions with respect to the amino acid sequence of SEQ ID NO: 10.

16. The protein of claim 15, wherein said protein has SAg activity.

17. A protein which is 98.0% to 99.9% identical to the amino acid sequence of SEQ ID NO: 7 over a length of 153 amino acids.

18. The protein of claim 17, wherein said protein has SAg activity.

19. A protein comprising the amino acid sequence of SEQ ID NO: 1, wherein Xaa₉₇, Xaa₁₅₄, Xaa₂₇₂, Xaa₃₄₈, Xaa₅₃₄ are chosen from the following amino acids:

| | |
|---|---|
| Xaa₉₇: | Tyr, Cys, Phe, Ser |
| Xaa₁₅₄: | Trp, Leu, Ser, Stop |
| Xaa₂₇₂: | Val, Ile, Leu |
| Xaa₃₄₈: | Val, Ile, Leu, Phe |
| Xaa₅₃₄: | Val, Ile, Leu, Phe |

provided that when Xaa₁₅₄ is STOP, Xaa₉₇ is not Tyr; and when Xaa₁₅₄ is Trp and each of Xaa₂₇₂, Xaa₃₄₈, Xaa₅₃₄ is Val, Xaa₉₇ is not Cys.

20. The protein of claim 19, wherein said protein has SAg activity.

21. The protein of claim 19, wherein said superantigen activity is specific for Vβ7 and/or Vβ13 chains.

22. A protein or peptide comprising a fragment of SEQ ID NO: 1, wherein fragment is 6 to 556 amino acids long and includes the portion spanning at least one of positions 154, 272, 348, 534 of SEQ ID NO: 1, wherein Xaa₉₇, Xaa₁₅₄, Xaa₂₇₂, Xaa₃₄₈, Xaa₅₃₄ are selected from the following amino acids:

| | |
|---|---|
| Xaa97: | Tyr, Cys, Phe, Ser |
| Xaa154: | Trp, Leu, Ser, Stop |
| Xaa272: | Val, Ile, Leu |
| Xaa348 | Val, Ile, Leu, Phe |
| Xaa534: | Val, Ile, Leu, Phe |

provided that when Xaa154 is STOP, Xaa97 is not Tyr; and that when Xaa154 is Trp, and each of Xaa272, Xaa348, Xaa534 is Val, Xaa97 is not Cys.

23. A protein or peptide comprising a fragment of SEQ ID NO: 9 or SEQ ID NO: 10, wherein said fragment is 6 to 556 amino acids long and includes the portion spanning at least one of positions 154, 272, 348, 534 of SEQ ID NO: 9 or SEQ ID NO: 10.

24. The protein or peptide of claim 22, having 10 to 300 amino acids.

25. The protein or peptide of claim 24, having 12 to 100 amino acids.

26. The protein or peptide of claim 25, having 15 to 30 amino acids.

27. The protein or peptide of claim 22, wherein said protein or peptide has SAg activity.

28. The protein or peptide of claim 27, wherein said SAg activity is specific for Vβ7 and/or Vβ13 chains.

29. The protein or peptide of claim 22, wherein said protein or peptide has no substantial Vβ7 and/or Vβ13 SAg activity.

30. A nucleic acid molecule comprising SEQ ID NO: 14.

31. A nucleic acid molecule having from 1 to 15 nucleotide substitutions, deletions and/or insertions with respect to SEQ ID NO: 13.

32. A nucleic acid molecule comprising SEQ ID NO: 10.

33. A nucleic acid molecule comprising a fragment of SEQ ID NO: 9, where said fragment is 16 to 1668 nucleotides long, including the nucleotides encoding the amino acids at positions 97, 154, 272, 348, 534 of SEQ ID NO: 9 or SEQ ID NO: 10.

34. The nucleic acid molecule of claim 33, which is 30 to 900 nucleotides long.

35. The nucleic acid molecule of claim 34, which is 60 to 500 nucleotides long.

36. The nucleic acid molecule of claim 35, which is 75 to 300 nucleotides long.

37. A nucleic acid complement to the nucleic acid molecule of claim 30.

38. A nucleic acid complement to the nucleic acid molecule of claim 31.

39. A nucleic acid complement to the nucleic acid molecule of claim 32.

40. The nucleic acid molecule of claim 30, wherein said molecule is DNA.

41. The nucleic acid molecule of claim 31, wherein said molecule is DNA.

42. The nucleic acid molecule of claim 32, wherein said molecule is DNA.

43. The nucleic acid molecule of claim 30, wherein said molecule is RNA.

44. The nucleic acid molecule of claim 31, wherein said molecule is RNA.

45. The nucleic acid molecule of claim 32, wherein said molecule is RNA.

46. A nucleic acid molecule comprising SEQ ID NO: 15.

47. A nucleic acid molecule comprising SEQ ID NO: 17.

48. A nucleic acid molecule comprising SEQ ID NO: 18.

49. A nucleic acid molecule comprising SEQ ID NO: 20.

50. A nucleic acid molecule suitable for use as a primer in a nucleic acid amplification reaction, wherein said molecule is 30 to 300 nucleotides long, and has a sequence common to SEQ ID NOs: 15-20, or a sequence complementary thereto.

51. The nucleic acid molecule of claim 50, wherein said sequence is identical or complementary to SEQ ID NOs: 15-17 between positions 1-173, 195-278, 329-620, 651-698, 700-845.

52. The nucleic acid molecule of claim 50, wherein said sequence is identical or complementary to SEQ ID NOs: 18-20 between positions 20-300, 305460, 505-770.

53. A pair of nucleic acid molecules suitable for use as primers in genotyping of the HERV K18 locus, including
the nucleic acid molecule of claim 51, and
a nucleic acid molecule having a sequence identical or complementary to a portion of intron I of the CD48 gene, and having a length of 25 to 300 nucleotides.

54. A pair of nucleic acid molecules suitable for use as primers in genotyping of the HERV K18 locus, including
the nucleic acid molecule of claim 52, and
a nucleic acid molecule having a sequence identical or complementary to a portion of intron I of the CD48 gene, and having a length of 25 to 300 nucleotides.

55. A method for genotyping the human HERV K-18 locus, comprising the steps of
analyzing at least one of the polymorphic regions of HERV-K18 in both chromosomes of an individual, said polymorphic region selected from the group consisting of the ENV region, the 5′ LTR and the 3′ LTR, so the sequence of said region is determined, and
assigning a genotype on the basis of the sequence identified in the polymorphic region.

56. The method of claim 55, wherein the analyzing at least one of the polymorphic regions comprises:
a. selecting a pair of nucleic acid primers suitable for amplifying a region of the HERV K18 locus, said region being chosen from:
i. at least a portion of the env region of HERV K18, said portion encoding amino acids 97 to 154 of SEQ ID NOs: 7-10.
ii. the 5′ LTR of HERV K18, or
iii. the 3′ LTR of HERV-K18;
b. amplifying genomic DNA of a sample from a subject, and
c. determining the DNA sequence of the amplified fragment(s).

57. The method of claim 56, wherein at least one of the primers is unique to the HERV K18 locus.

58. The method of claim 57, wherein the primers are suitable for amplification of the whole env region of HERV K18.

59. The method of claim 55, wherein the forward primer corresponds to all or part of the 5′ untranslated region of HERV K18 env.

60. The method of claim 55, wherein one of the primers corresponds to all or part of intron I of the CD48 gene.

61. The method of claim 55, wherein the primers correspond to genomic sequences which are less than 12 kb apart.

62. The method of claim 61, wherein the primers correspond to genomic sequences which are less than 5 kb apart.

63. The method of claim 62, wherein the reverse primer corresponds to a portion of intron 1 of the CD48 gene, said portion flanking HERV K18 on the 3′ side in the genome, said portion having a length of 30 to 300 nucleotides.

64. The method of claim 55, further comprising the steps of genotyping the subject for genetic diversity at at least one additional locus.

65. The method of claim 64, wherein the additional locus or loci is/are associated with autoimmune disease.

66. The method of claim 55, wherein the additional locus or loci is chosen from at least one of the following:
the TCRβV locus;
an HLA class II locus (IDDM1); and
the INS locus (IDDM2).

67. The method of claim 66, wherein the additional loci include the TCRβV locus and the genotyping comprises determination of the presence or absence of the Vβ7.2 and/or the Vβ13.2 gene.

68. The method of claim 66, wherein the additional loci include an HLA Class II locus and the genotyping comprises determination of the allelic variation of at least one DR gene and/or at least one DQ gene, and/or at least one DP gene.

69. The method of claim 66, wherein the additional loci include the INS (IDDM2) locus.

70. The method of claim 66, comprising the steps of genotyping the subject for genetic diversity at three or more loci.

71. A method for identifying individuals at risk for IDDM, said method comprising a combined genotyping of the HERV-K18 locus with at least one of the TCRβV, IDDM1 and IDDM2 loci.

72. An antibody recognizing a polypeptide selected from the group consisting of SEQ ID NO: 6-10.

73. The antibody of claim 72, wherein said antibody is a monoclonal antibody.

74. The antibody of claim 72, wherein said antibody is a polyclonal antibody.

75. An antibody recognizing a polypeptide comprising SEQ ID NO: 1.

76. The antibody of claim 75, wherein said antibody is a monoclonal antibody.

77. The antibody of claim 75, wherein said antibody is a polyclonal antibody.


SEQUENCE LISTING (46 sequences)

SEQ ID NO: 1 - protein, 560 aa, Human endogenous retrovirus
  VARIANT (97): Where Xaa is Tyr, Cys ,Phe or Ser
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNXLQDFSYQRSLKFRPKGKTCPKEIPKGSKNTEVLVWEECVANSVVILQNNEFGTIIDXAPRGQFYHNCSGQTQSCPSAQVSPAVDSDLTESLDKHKHKKLQSFYLWEWEEKGISTPRPKIISPVSGPEHPELWRLTVASHHIRIWSGNQTLETRYRKPFYTIDLNSILTVPLQSCXKPPYMLVVGNIVIKPASQTITCENCRLFTCIDSTFNWQHRILLVRAREGMWIPVSTDRPWEASPSIHILTEILKGXLNRSKRFIFTLIAVIMGLIAVTATAAVAGVALHSSVQSVNFVNYWQKNSTRLWNSQSSIDQKLASQINDLRQTVFWMGDRLMTLEHHFQLQCDWNTSDFCITPQIYNESEHHWDMVRRHLQGREDNLTLDISKLKEQIFEASKAHLNLVPGTEAIAGVADGLANLNPVTWIKTIRSTMIINLILIXVCLFCLLLVCRCTQQLRRDSDIENGP

SEQ ID NO: 2 - DNA, 2689 bp, Human endogenous retrovirus
atggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattatttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattaggcacctcgaggtcaattctaccacaattgctcaggacaaactcagtcgtgtccaagtgcacaagtgagtccagctgtcgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcctaaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcgttttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcgttgtgtgcctgttttgtctgttgttagtctgcaggtgtacccaacagctccgaagagacagtgacatcgagaacgggccatgatgacgatggcggttttgtcgaaaagaaaagggggaaatgtggggaaaagcaagagagatgagattgttactgtgtctgtatagaaagaagtagacataggagactccattttgttctgtactaagaaaaattcttctgccttgagatgctgttaatctatgaccttacccccaaccccgtgctctctgaaacatgtgctgtgtcaaactcagggttaaatggattaagggtggtgcaagatgtgctttgttaaacagatgcttgaaggcagcatgctcattaagagtcatcaccactccctaatctcaagtacccagggacacaaacactgcgaaaggccgcagggacctctgcctaggaaagccaggtattgtccaaggtttctccccatgtgatagtctgaaatatggcctcgtgggaagggaaagacctgaccatcccccagaccaacacccgtaaagggtctgtgctgaggaggattagtataagaggaaagcatgcctcttgcagttgagagaagaggaagacatctgtctcctgcccatccctgggcaatggaatgtctcagtataaaacccgattgaacattccatctactgagatagggaaaaactgccttagggctggaggtgggacatgtgggcagcaatactgctttgtaaagcattgagatgtttatgtgtatgtatatctaaaagcacagcacttgatcctttaccttgtctatgatgcaaacacctttgttcacgtgtttgtctgctgaccctctccccactattgtcttgtgaccctgacacatccccctctcggagaaacacccacgaatgatcaataaatactaagggaactcagaggctggcgggatcctccatatgctgaacgctggttccccgggcccccttatttctttctctatactttgtctctgtgtctttttcttttccaagtctctcattccaccttatgagaaacacccacaggtgtggaggggcaacccaccccttca

SEQ ID NO: 3 - DNA, 1683 bp, Human endogenous retrovirus
atggtaacaccagtcacatggatgggtaatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattgtttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattgggcacctcgaggtcaattctaccacaattgctcaggacaaactcagtcgtgtccaagtgcacaagtgagtccagctgtcgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcgtaaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcgttttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcgttgtgtgcctgttttgtctgttgttagtctgcaggtgtacccaacagctccgaagagacagtgacatcgagaacgggccatga

SEQ ID NO: 4 - DNA, 2689 bp, Human endogenous retrovirus
atggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattatttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattgggcacctcgaggtcaattctaccacaattgctcaggacaaactcagtcgtgtccaagtgcacaagtgagtccagctgttgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcataaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcattttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcattgtgtgcctgttttgtctgttgttagtctgcaggtgtacccaacagctccgaagagacagtgacatcgagaacgggccatgatgacgatggcggttttgtcgaaaagaaaagggggaaatgtggggaaaagcaagagagatgagattgttactgtgtctgtatagaaagaagtagacataggagactccattttgttctgtactaagaaaaattcttctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaacactgcgaaagaccgcagggacctctgcctaggaaagctaggtattgtccaaggtttctccccatgtgatagtctgaaatatggcctcgtgggaagggaaagacctgaccatcccccagaccaacacccgtaaagggtctgtgctgaggaggattagtataagaggaaagcatgcctcttgcagttgagagaagaggaagacatctgtctcctgcccatccctgggcaatggaatgtctcagtataaaacccgattgaacattccatctactgagatagggaaaaactgccttagggctggaggtgggacatgtgggcagcaatactgctttgtaaagcattgagatgtttatgtgtatgcatatctaaaagcacagcacttgatcctttaccttgtctatgatgcaaagacctttgttcacgtgtttgtctgctgaccctctccccactattgtcttgtgaccctgacacatccccctctcggagaaacacccacgaatgatcaataaatactaagggaactcagaggctggcgggatcctccatatgctgaacgttggttccccgggcccccttatttctttctctatactttgtctctgtgtctttttcttttccaagtctctcgttccaccttatgagaaacacccacaggtgtggaggggcaacccaccccttca

SEQ ID NO: 5 - DNA, 46 bp, Artificial Sequence
  Description of Artificial Sequence oligonucleotide primer
attgcggccgctcagtcgaccccaaacctttaaatattgtctcatg

SEQ ID NO: 6 - DNA, 61 bp, Human endogenous retrovirus
acatttgaagttctacaatgaacccatcagagatgcaaagaaaagcgcctccacggagatg

SEQ ID NO: 7 - protein, 153 aa, Human endogenous retrovirus
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNYLQDFSYQRSLKFRPKGKTCPKEIPKGSKNTEVLVWEECVANSVVILQNNEFGTIID

SEQ ID NO: 8 - protein, 560 aa, Human endogenous retrovirus
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNCLQDFSYQRSLKFRPKGKTCPKEIPKGSKNTEVLVWEECVANSVVILQNNEFGTIIDWAPRGQFYHNCSGQTQSCPSAQVSPAVDSDLTESLDKHKHKKLQSFYLWEWEEKGISTPRPKIISPVSGPEHPELWRLTVASHHIRIWSGNQTLETRYRKPFYTIDLNSILTVPLQSCVKPPYMLVVGNIVIKPASQTITCENCRLFTCIDSTFNWQHRILLVRAREGMWIPVSTDRPWEASPSIHILTEILKGVLNRSKRFIFTLIAVIMGLIAVTATAAVAGVALHSSVQSVNFVNYWQKNSTRLWNSQSSIDQKLASQINDLRQTVFWMGDRLMTLEHHFQLQCDWNTSDFCITPQIYNESEHHWDMVRRHLQGREDNLTLDISKLKEQIFEASKAHLNLVPGTEAIAGVADGLANLNPVTWIKTIRSTMIINLILIVVCLFCLLLVCRCTQQLRRDSDIENGP

SEQ ID NO: 9 - protein, 560 aa, Human endogenous retrovirus
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNYLQDFSYQRSLKFRPKGKTCPKEIPKGSKNTEVLVWEECVANSVVILQNNEFGTIIDWAPRGQFYHNCSGQTQSCPSAQVSPAVDSDLTESLDKHKHKKLQSFYLWEWEEKGISTPRPKIISPVSGPEHPELWRLTVASHHIRIWSGNQTLETRYRKPFYTIDLNSILTVPLQSCIKPPYMLVVGNIVIKPASQTITCENCRLFTCIDSTFNWQHRILLVRAREGMWIPVSTDRPWEASPSIHILTEILKGILNRSKRFIFTLIAVIMGLIAVTATAAVAGVALHSSVQSVNFVNYWQKNSTRLWNSQSSIDQKLASQINDLRQTVFWMGDRLMTLEHHFQLQCDWNTSDFCITPQIYNESEHHWDMVRRHLQGREDNLTLDISKLKEQIFEASKAHLNLVPGTEAIAGVADGLANLNPVTWIKTIRSTMIINLILIIVCLFCLLLVCRCTQQLRRDSDIENGP

SEQ ID NO: 10 - protein, 560 aa, Human endogenous retrovirus
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNYLQDFSYQRSLKFRPKGKTCPKEIPKGSKNTEVLVWEECVANSVVILQNNEFGTIIDWAPRGQFYHNCSGQTQSCPSAQVSPAVDSDLTESLDKHKHKKLQSFYLWEWEEKGISTPRPKIISPVSGPEHPELWRLTVASHHIRIWSGNQTLETRYRKPFYTIDLNSILTVPLQSCVKPPYMLVVGNIVIKPASQTITCENCRLFTCIDSTFNWQHRILLVRAREGMWIPVSTDRPWEASPSIHILTEILKGVLNRSKRFIFTLIAVIMGLIAVTATAAVAGVALHSSVQSVNFVNYWQKNSTRLWNSQSSIDQKLASQINDLRQTVFWMGDRLMTLEHHFQLQCDWNTSDFCITPQIYNESEHHWDMVRRHLQGREDNLTLDISKLKEQIFEASKAHLNLVPGTEAIAGVADGLANLNPVTWIKTIRSTMIINLILIIVCLFCLLLVCRCTQQLRRDSDIENGP

SEQ ID NO: 11 - DNA, 1683 bp, Human endogenous retrovirus
atggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattatttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattaggcacctcgaggtcaattctaccacaattgctcaggacaaactcagtcgtgtccaagtgcacaagtgagtccagctgtcgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcctaaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcgttttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcgttgtgtgcctgttttgtctgttgttagtctgcaggtgtacccaacagctccgaagagacagtgacatcgagaacgggccatga

SEQ ID NO: 12 - DNA, 1683 bp, Human endogenous retrovirus
atggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattatttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattaggcacctcgaggtcaaatctaccacaattgctcaggacaaactcagtcgtgtccaagtgcacaagtgagtccagctgtcgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcctaaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcgttttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcgttgtgtgcctgttttgtctgttgttagtctgcaggtgtacccaacagctccgaagagacagtgacatcgagaacgggccatga

SEQ ID NO: 13 - DNA, 1683 bp, Human endogenous retrovirus
atggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattgtttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattgggcacctcgaggtcaattctaccacaattgctcaggacaaactcagtcgtgtccaagtgcacaagtgagtccagctgtcgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcgtaaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcgttttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcgttgtgtgcctgttttgtctgttgttagtctgcaggtgtacccaacagctccgaagagacagtgacatcgagaacgggccatga

SEQ ID NO: 14 - DNA, 1683 bp, Human endogenous retrovirus
atggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattatttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattgggcacctcgaggtcaattctaccacaattgctcaggacaaactcagtcgtgtccaagtgcacaagtgagtccagctgttgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcataaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcattttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcattgtgtgcctgttttgtctgttgttagtctgcaggtgtacccaacagctccgaagagacagtgacatcgagaacgggccatga

SEQ ID NO: 15 - DNA, 975 bp, Human endogenous retrovirus
tgtggggaaaagcaagagagatgagattgttactgtgtctgtatagaaagaagtagacataggagactccattttgttctgtactaagaaaaattcttctgccttgagatgctgttaatctatgaccttacccccaaccccgtgctctctgaaacatgtgctgtgtcaaactcagggttaaatggattaagggtggtgcaagatgtgctttgttaaacagatgcttgaaggcagcatgctcattaagagtcatcaccactccctaatctcaagtacccagggacacaaacactgcgaaaggccgcagggacctctgcctaggaaagccaggtattgtccaaggtttctccccatgtgatagtctgaaatatggcctcgtgggaagggaaagacctgaccatcccccagaccaacacccgtaaagggtctgtgctgaggaggattagtataagaggaaagcatgcctcttgcagttgagagaagaggaagacatctgtctcctgcccatccctgggcaatggaatgtctcagtataaaacccgattgaacattccatctactgagatagggaaaaactgccttagggctggaggtgggacatgtgggcagcaatactgctttgtaaagcattgagatgtttatgtgtatgtatatctaaaagcacagcacttgatcctttaccttgtctatgatgcaaacacctttgttcacgtgtttgtctgctgaccctctccccactattgtcttgtgaccctgacacatccccctctcggagaaacacccacgaatgatcaataaatactaagggaactcagaggctggcgggatcctccatatgctgaacgctggttccccgggcccccttatttctttctctatactttgtctctgtgtctttttcttttccaagtctctcattccaccttatgagaaacacccacaggtgtggaggggcaacccaccccttcatgagac

SEQ ID NO: 16 - DNA, 975 bp, Human endogenous retrovirus
tgtggggaaaagcaagagagatgagattgttactgtgtctgtatagaaagaagtagacataggagactccattttgttctgtactaagaaaaattcttctgccttgagatgctgttaatctatgaccttacccccaaccccgtgctctctgaaacatgtgctgtgtcaaactctgggttaaatggattaagggtggtgcaagatgtgctttgttaaacagatgcttgaaggcagcatgctcattaagagtcatcaccactccctaatctcaagtacccggggacacaaacactgcgaaaggccgcagggacctctgcctaggaaagccaggtattgtccaaggtttctccccatgtgatagtctgaaatatggcctcgtgggaagggaaagacctgaccatcccccagaccaacacccgtaaagggtctgtgctgaggaggattagtataagaggaaagcatgcctcttgcagttgagagaagaggaagacatctgtctcctgcccatccctgggcaatggaatgtctcagtataaaacccgattgaacattccatctactgagatagggaaaaactgccttagggctggaggtgggacatgtgggcagcaatactgcttcgtaaagcattgagatgtttatgtgtatgcatatctaaaagcacagcacttgatcctttaccttgtctatgatgcaaacacctttgttcacgtgtttgtctgctgaccctctccccactattgtcttgtgaccctgacacatccccctctcggagaaacacccacgaatgatcaataaatactaagggaactcagaggctggcgggatcctccatatgctgaacgctggttccccgggcccccttatttctttctctatactttgtctctgtgtctttttcttttccaagtctctcattccaccttatgagaaacacccacaggtgtggaggggcaacccaccccttcatgagac

SEQ ID NO: 17 - DNA, 975 bp, Human endogenous retrovirus
tgtggggaaaagcaagagagatgagattgttactgtgtctgtatagaaagaagtagacataggagactccattttgttctgtactaagaaaaattcttctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaacactgcgaaagaccgcagggacctctgcctaggaaagctaggtattgtccaaggtttctccccatgtgatagtctgaaatatggcctcgtgggaagggaaagacctgaccatcccccagaccaacacccgtaaagggtctgtgctgaggaggattagtataagaggaaagcatgcctcttgcagttgagagaagaggaagacatctgtctcctgcccatccctgggcaatggaatgtctcagtataaaacccgattgaacattccatctactgagatagggaaaaactgccttagggctggaggtgggacatgtgggcagcaatactgctttgtaaagcattgagatgtttatgtgtatgcatatctaaaagcacagcacttgatcctttaccttgtctatgatgcaaagacctttgttcacgtgtttgtctgctgaccctctccccactattgtcttgtgaccctgacacatccccctctcggagaaacacccacgaatgatcaataaatactaagggaactcagaggctggcgggatcctccatatgctgaacgttggttccccgggcccccttatttctttctctatactttgtctctgtgtctttttcttttccaagtctctcgttccaccttatgagaaacacccacaggtgtggaggggcaacccaccccttcatgagac

SEQ ID NO: 18 - DNA, 969 bp, Human endogenous retrovirus
tgtggggaaaagcaagagaggtcagattgttactgtgtctgtatagaaagaagtagacataggagactccattttgttctgtactaagaaaaattattctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaaaactgcggaagcctgcaggggcctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtataagaggaaagcatgtctcttgcagttgagacaagaagaaggcatctgtttcccgcccatccctgggcaatggaatgtctcggtataaaacccgattgtacgttccacctactgagatagggagaaaccaccttagggctggaggtgggacatgcaggcagcaatactgctttgtaaagcattgagatgtttatgtgtatgcatatctaaaagcacagcatttaatcctttaccttgtctatgatgcaaagacctttgttcacgtgtttgtctgctcaccctctccccactattgtcttgtgaccctgacacatctccctctaggagaaacacccacgaatgatcaataaatactaaggggactcagaggctggtgggatcctccatatgctgaacgttggttccccgggcccccttatttctttctctatactttgtctctgtgtctttttcttttccaagtctctcattgcaccttacgagaaacacccacaggtgtggaggggcaacccaccccttca

SEQ ID NO: 19 - DNA, 1010 bp, Human endogenous retrovirus
tgtggggaaaagcaacagaggtcagattgttactgtgtctgtatagaaagaagtagacataggagactccattttgttctgtactaagaaaaattattctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaaaactgcggaaggctgcaggggcctctgcctaggaaagccaggtattgtccaaggtttctccccatgtgagagtctgaaatatggcctcgtgggaagggaaagacctgaccgtcccccagcccgacacccataaagggtctgtgctgaggaggattagtataagaggaaagcatgcctcttgcagttgagacaagaggaaggcatctgtttcccacccatccctgggcaatggaatgtctcggtataaaacccgattgtacgttccacctactgagatagggagaaaccaccttagggctggaggtgggacatgcaggcagcaatactgctttgtaaagcattgagatgtttatgtgtatgcatatctaaaagcacagcatttaatcctttaccttgtctatgatgcaaagacctttgttcacgtgtttgtctgctcaccctctccccactattgtcttgtgaccctgacacatctccctctcagagaaacacccacgaatgatcaataaatactaaggggactcagaggctggtgggatcctccatatgctgaacgttggttccccgggcccccttatttctttctctatactttgtctctgtgtctttttcttttccaagtctctcattgcaccttacgagaaacacccacaggtgtggaggggcaacccaccccttcatctggtgcccaacgtggaggcttttctctggggtgaaggta

SEQ ID NO: 20 - DNA, 972 bp, Human endogenous retrovirus
tgtggggaaaagcaagagaggtcagattgttactgtgtctgtatagaaagaagtagacataggagactccattttgttctgtactaagaaaaattattctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaaaactgcggaagcctgcaggggcctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtataagaggaaagcatgtctcttgcagttgagacaagaggaaggcatctgtttcccgcccatccctgggcaatggaatgtctcggtataaaacccgattgtacgttccacctactgagatagggagaaaccaccttagggctggaggtgggacatgcaggcagcaatactgctttgtaaagcattgagatgtttatgtgtatgcatatctaaaagcacagcatttaatcctttaccttgtctatgatgcaaagacctttgttcacgtgtttgtctgctcaccctctccccactattgtcttgtgaccctgacacatctccctctcggagaaacacccacgaatgatcaataaatactaaggggactcagaggctggtgggatcctccatatgctgaacgttggttccccgggcccccttatttctttctctatactttgtctctgtgtctttttcttttccaagtctctcattgcaccttacgagaaacacccacaggtgtggaggggcaacccaccccttcatct

SEQ ID NO: 21 - DNA, 233 bp, Human endogenous retrovirus
gcaccttacgagaaacacccacaggtgtggaggggcaacccaccccttcatttggtgcccaacgtggaggcttttctctggggtgaaggtacactcgagcgtggtcattgaggacaagtcgacaagagatcccgagtacatttacagtcagccttacggtaagcctgtgcactcggaagaaggtagggtgacaatggggcaaactaaaactaaaagtaaatatgcctgtcatt

SEQ ID NO: 22 - DNA, 44 bp, Human endogenous retrovirus
atcagatctaacactagtcccatcagacacacaagcagctgggc

SEQ ID NO: 23 - DNA, 46 bp, Human endogenous retrovirus
attgcggccgctcagtcgactcccaggggcaggctcatcaacattg

SEQ ID NO: 24 - DNA, 43 bp, Human endogenous retrovirus
atcagatctaacactagtgttgctgagagggatcctgaaagat

SEQ ID NO: 25 - DNA, 45 bp, Human endogenous retrovirus
attgcggccgctcagtcgaccttggcacactgttgttttcaaccc

SEQ ID NO: 26 - DNA, 6020 bp, Human endogenous retrovirus
aggtaggactagtaggtgttggaaaggccgaggaaatttatcagagcacatttatcttgctttgcactggccctgatggtcaaaaaggtacaattcagccctatatcatgccaattcacattaatctttggggtagagatttactggcaaaatagagggctgaaattaatattccacataactcttctagtgctcccagtcagcatatgatggaaaatataaggtttgttcctggattaccaaacccctcccagtcactataaaagaaaacagggctggtttaggttattctttttagtggcagccactgccatacctcctgatcccattcccttacaatggaaacctaaaactcccgtttaggttcagcagtggccgctttctaaagaaaaactggaggctttaaatcaattggtttctgagcagttgcaacttggatatgtggaacattctctttccccttagaattctcctgtgttcctagtaaaaaagaaatcaggcaaatggcggatggtaaccgatttaagggccattaatgctgtaattaaacctatgggggccgtccaacctggctttaatacctaaaaattagcctctcatagttattgatcttaaagatttttttttatattgctttacataaatcagattgtgaaaaatttgcttttactgtaccatctatcaataatcaggaacctgcagttcattatcaatggaaagtacttcctcaaggaatgctaaatagccctacaatctgccagctttatgttgggcaagtgctttcaccagttcaagcccaatttcccgaggcctatattcatcattatattgatgatattttaattgctgcccccactgataaagaattgactgttaccaaattttgagctgctgtgttatagaggctggattacacattgctcaagataaaattcatcagaccactcctgttcaatatttaggaatggtggtcgataaacaatgtattcaacctcaaaaagttcaaattaggagagattctttaaaaactttagatgacttccacaaacttttaggtaacattaattatttaagacctactttaggcattccaacctatgcactgtctaacttgatttctatgttgcggggagattccaatctccacagcgccaggattttgacctctgaggctttaatagaactggaatttgtagaagaaagaatccagactgcccagttatctagagtacagccatttcagccttttcagcttctagtttttgcttcattacactcccctactggactaatagttcaacataatgatttagtggagtgatgttttcttccttattctgtctcaaaaactttgtctgtttatctagaccaaatagccatattaattagacaggcttggtgcagaatacttcaaatttctggatttgatccaaatataattgtagttcctttaaattggctcaaagttcaagctgcctttcaacattctgtactgtggcaaattcacttggctgattttattggcgttattgacaatcattatccaaaaaacaaattatttgattttataaaaatgacttcttaggtggttcctcgattaaccaaaaatcaacccattcctgaggccgttacagtgttcactgatggctccagtaatggcaatgctggctatgtaagtcctacagacaaacttatttctacctcttatacttctgctcaaaaggcggagttaattgctgtgattactgccttacaggatttccccaaacctttaaatattgtctcatgaaggggtgggttgcccctccacacctgtgggtgtttctcataaggtggaacgagagacttggaaaagaaaaagacacagagacaaagtatagagaaagaaataagggggcccggggaaccaacgttcagcatatggaggatcccgccagcctctgagttcccttagtatttattgatcattcgtgggtgtttctccgagagggggatgtgtcagggtcacaagacaatagtggggagagggtgagcagacaaacacgtgaacaaaggtctttgcatcatagacaaggtaaaggatcaagtgctgtgcttttagatatgcatacacataaacatctcaatgctttacaaagcagtattgctgcccacatgtcccacctccagccctaaggcagtttttccctatctcagtagatggaatgttcaatcgggttttatactgagacattccattgcccagggatgggcaggagacagatgtcttcctcttctctcaactgcaagaggcatgctttcctcttatactaatcctcctcagcacagaccctttacgggtgttggtctgggggatggtcaggtctttcccttcccacgaggccatatttcagactatcacatggggagaaaccttggacaatacctagctttcctaggcagaggtccctgcggtctttcgcagtgtttgtgtccctgggtacttgagattagggagtggtgatgactcttaatgagcatgctgccttcaagcatctgtttaacaaagcacatcttgcaccgcccttaatccatttaaccctgagtttgacacagcacatgtttcagagagcacggggttgggggtaaggtcatagattaacagcatctcaaggcagaagaatttttcttagtacagaacaaaatggagtctcctatgtctacttctttctatacagacacagtaacaatctcatctctcttgcttttccccacatttcccccttttcttttcgacaaaaccgccatcgtcatcatggcccgttctcgatgtcactgtctcttcggagctgttgggtacacctgcagactaacaacagacaaaacaggcacacaatgattaatatgagatttataatcatagtacttctgatggtcttaatccaagtgacagggttaagatttgcgaggccatcagcaactcctgcaattgcctcagttcctggcaccaaatttaaatgggcttttgatgcttcgaaaatttgttcttttaatttggaaatgtctaaagtgagattatcttctcttccctgtagatggcgtctaaccatgtcccagtgatgctcagactcattataaatttggggtgtaatacaaaaatctgacgtattccagtcacactgtaactggaaatgatgttctaaggtcatgagcctgtctcccatccaaatgacagtttgtctaagatcattaatttgacttgccaatttttgatcaatactagattgtgaattccacaatcttgtagaattcttttgccaataattaacaaagtttactgactgaacagaagagtgcaatgcaactcctgccacagcagccgtagctgtgactgcaattaatcccataatcactgcaattaaagtaaaaatgaatcttttggatctatttaaaatgccttttaatatttcagtcaaaatatggatggatggcgaggcctcccacggtcggtccgtggacacagggatccacatgccttctcttgctctcaccagcagaatacggtgctgccaattaaaagttgaatcaatgcaagtaaacaatctacaattttcacaggttatagtttgggaggctggtttaataactatatttcctacaactagcatataagggggctttatgcaactttgtaaaggaaccgttagaatggaatttaggtcgatagtataaaatggcttacgatatcttgtttctaaagtttgatttccagaccaaattctaatgtggtgtgaggccacagtaagcctccacaattctggatgttcaggaccagaaacaggacttattatttttggtcttggggtagagattcctttttcttcccattcccaaaggtagaaagactgtaattttttatgcttatgtttgtctagactttctgttaagtcgctatcaacagctggactcacttgtgcacttggacacgactgagtttgtcctgagcaattgtggtagaattgacctcgaggtgcccaatctataatagtttcgaattcattgttttgtaatatcaccacactattggccacacattcttcccaaactaaaacttctgtattctttgatcctttaggaatttccttggggcaagttttccctttaggtctaaattttaatgatctttgataagaaaagtcttgtaaataatttacccgtggcctgagtgacatcccgcttaccatgtgataagtgaatctactgttaggactgacagtaggtacttctaccaaccaattttggactgcaggcattaaacatcctggtgctctccctaggcaaataggaggataatgatacccaatggaaatatttatcatcatcccttcttcctcaggtttggcagggcagcgatcatctgtggggccaggtacccatacactatcattaacatatacttctataggattatccatccatgtgactggtgttaccatctccgtggaggcgcttttctttgcatctctgatgggttcattgtagaacttcaaatgtctagtgggtatccaaacaggaagctgattttctcctggtgaaacacaagcaaaacctctcccccacgttatcagcttccctatttcccatgtcttattttaattatctttccactaaattagttttccttcatgtgggctgttctttttaccagtaagatgttctgcagaagtagtagtctgatttctataaatgtttaaaaaatttaaagtatagagtgctagattaagttgcatctgaggagtggtacactccttactgtctcccccttctttttgtttaactaattgagttttgagtgttctattagttctttcaactatggcctgtccttgggaattataaggaattcctgttgtatgtgaaattttccactgacttaagaatttttggaaagctttactacaatatcctggtccattgtcagttttgattttttctggaactcccattacagcaaaacaagacaataaatgttttttaacatgggaagtactttctcctgtttggcaagttgcccacatgaaatgtgaataagtatcaactgttacatgacatatgataatcttccaaatgaaggtacatgcgtgacatccatttgccataatgcattaggacacagacctctgggttaactcctgcctcttgagtgggcaggtctaagacttgacactgggtgcaatgttgtacaatatcttttgcctgtttctatgtgacatcaaatttgttttttaatcctgctgcatttacatgagtcaaagcatgaagttcttgtgcttttatgaatgcagatgataccagtaagtcagcttgttcatttgctttagtcaaaggccctggtaaattagtgtgtgctcgaatatgagtaatataaaatgggaagtttctttttcttacagtttgttgtaataaattgaatagctggtttaactgatcgtccatgctatatttaattagagctgtctcaacatcccttgtagcctgtactacatatgcagaatctgatataatattgataggttgatcaaaatcttgtaacactgtaatgactgcaaccaactctgctctttgagccgattgatatggagttttgattactcgttcttttggccctgtgtaagccacttttccattgctggaaccatcagtaaatactgttagagcattttctaaaggttcacatctggtaattttaggtagaatccaagtagtcaattttaagaactggaagatttttgtttttgggtaatgattatcaataattcccacaaaattagcaagaccaatctgccatgcaccagaattgataaaggcttgtctaacttgttccttggttaaagggacaactattttgtctgggtcatttccacataattttattattcgtaatcttgtcggaccaattaaagtagctatttgatccaagtacaatgtaaaagtcttaactgtactgtgaggaaggaatgaccactccacaagatcagtat

SEQ ID NO: 27 - DNA, 42 bp, Artificial Sequence
  Description of Artificial Sequence oligonucleotide primer
cacagatctagaactagtgccaccatgtgctccagaggttgg

SEQ ID NO: 28 - DNA, 24 bp, Artificial Sequence
  Description of Artificial Sequence oligonucleotide primer
ctgtcatttggatgggagacaggc

SEQ ID NO: 29 - DNA, 34 bp, Artificial Sequence
  Description of Artificial Sequence oligonucleotide primer
cacggatcccagattccgcttatgttgtacatgc

SEQ ID NO: 30 - DNA, 39 bp, Artificial Sequence
  Description of Artificial Sequence oligonucleotide primer
cacgtcgacggagaccacggttcatatgtaccaagtgac

SEQ ID NO: 31 - DNA, 42 bp, Artificial Sequence; identical to SEQ ID NO: 27
  Description of Artificial Sequence oligonucleotide primer

SEQ ID NO: 32 - DNA, 44 bp, Artificial Sequence
  Description of Artificial Sequence oligonucleotide primer
cacgcggccgcagagtcgactcaatcaatcaggtaagtaacagg

SEQ ID NO: 33 - DNA, 44 bp, Artificial Sequence
  Description of Artificial Sequence oligonucleotide primer
atcagatctaacactagtaacccatcagagatgcaaagaaaagc

SEQ ID NO: 34 - DNA, 46 bp, Artificial Sequence; identical to SEQ ID NO: 5
  Description of Artificial Sequence oligonucleotide primer

SEQ ID NO: 35 - DNA, 46 bp, Artificial Sequence
  Description of Artificial Sequence oligonucleotide primer
atcagatctaacactagttgccacactggtaacaccagtcacatgg

SEQ ID NO: 36 - DNA, 21 bp, Artificial Sequence
  Description of Artificial Sequence oligonucleotide primer
agaatgtgtggccaatagtgt

SEQ ID NO: 37 - DNA, 21 bp, Artificial Sequence
  Description of Artificial Sequence oligonucleotide primer
atggatggcgaggcctcccac

SEQ ID NO: 38 - DNA, 21 bp, Artificial Sequence
  Description of Artificial Sequence oligonucleotide primer
agagaaggcatgtggatccct

SEQ ID NO: 39 - DNA, 39 bp, Artificial Sequence
  Description of Artificial Sequence oligonucleotide primer
gacagatctcacactagtgctacagtgacatcgagaacg

SEQ ID NO: 40 - DNA, 20 bp, Artificial Sequence
  Description of Artificial Sequence oligonucleotide primer
cttcctgtttggatacccac

SEQ ID NO: 41 - DNA, 27 bp, Artificial Sequence
  Description of Artificial Sequence oligonucleotide primer
catgagacaatatttaaaggtttgggg

SEQ ID NO: 42 - DNA, 10569 bp, Human endogenous retrovirus
gtagttaaatcatgttttggttgttcagactgtttggacaattgggtttttaaagtgcgattggcctgttccaccacagcctgtccttgaggattgtaagggattctagtaatatgggaaattccttactgttgtataaatgaatcaaaagccttactaacatatccaggggcattgtctgtctttatttgatatggaagccccataactgcaaagcaagaatacagatgtttttaaaaatgggccgtgccttcccctgtttgtcaagtagcccagataaaacctgagaaggtatctactgagacatgcacatatgacagtctgccaaaggagctaacatgagtcacatccatttgccatcaagcattaggagttaggcctctaggcttaacaccaggttcctgatttggaagtacgaagacctggcactgagggcagctgtgaacaataaacttagcctgtttctaggtaagagcaaatttatcttttaagccagtggcattgacatgagtgagattatggaactcctgagcttcttgggtcattaaagacaccaaacagtcaacttcatggttaccagcagacatgggtcctggtaaagtggtatgagacctaatatgtgtaatataggaagggtgtctacactggtgaaccacctgttgtaaccttgaaaataaagaagccaattcagaattatcaatatgtttgatagtagcagtttctatatttttagtggcatgtacaacataagcggaatctgagactgtggggaaaagcaagagaggtcagattgttactgtgtctgtatagaaagaagtagacataggagactccattttgttctgtactaagaaaaattattctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaaaactgcggaagcctgcaggggcctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtataagaggaaagcatgtctcttgcagttgagacaagaggaaggcatctgtttcccgcccatccctgggcaatggaatgtctcggtataaaacccgattgtacgttccacctactgagatagggagaaaccaccttagggctggaggtgggacatgcaggcagcaatactgctttgtaaagcattgagatgtttatgtgtatgcatatctaaaagcacagcatttaatcctttaccttgtctatgatgcaaagacctttgttcacgtgtttgtctgctcaccctctccccactattgtcttgtgaccctgacacatctccctctcggagaaacacccacgaatgatcaataaatactaaggggactcagaggctggtgggatcctccatatgctgaacgttggttccccgggcccccttatttctttctctatactttgtctctgtgtctttttcttttccaagtctctcattgcaccttacgagaaacacccacaggtgtggaggggcaacccaccccttcatctggtgcccaacgtggaggcttttctctggggtgaaggtacactcgagcgtggtcattgaggacaagtcgacaagagatcccgagtacatctacagtcagccttacggtaagcttgtgcactcggaagaagctagggtgacaatggggcaaactaaaactaaaagtaaatatgcctcttatcttagcttcattaaaattcttttaaaaagagggggagttagagtatccaccaaaaatctaatcaagctatttcaaacaacagaacaattttgcccatggtttccagaacaaggaaatttagatctagaagattggaaaagaattggtaaggaactaaaacaagcaggtaggaagggtaatatcattccacttacagtatggaatgattggcccattattaaagcagctttagaaccatttcaaacagaagatagcgtttcagtttctgatgcccctggaagctgtataatagattgtaatgaaaagacaaggaaaaaatcccagaaggaaacggaaactttacattgcgaatatgtagcagagccgttaatggctcagtcaacgcaaaatgttgactataatcaattacaggaggtgatatatcctgaaacattaaaattagaaggaaaaggtccagaattagtggggccattagagtctaaaccacgagggccaagtcctctttcagcaggtcaggtgaccgtaacattacaacctcaagcgcaggttagagaaaataagacccaactgccagtagcttatcaatactggccaccggccgaacttcagtatcggccacccccagaaagtcagtatggatatctaggaatgcccccagcaccacagggcagggagccataccctcagccgcccactaggagacaatcctatggcaccacctagtagacagggtagtgaattacatgaaattattgagaagtcaagaaaggaaggagatactgaggcgtggcaattcccagtaacgttagaaccgatgccacctggagaaggagcccaagagggagagcctctcacagttgaggccagatacaagtctttttagataaaaatgctaaaagatatgaaagagggagtaaaacagtatggacccaactccccttatatgaggacattattagattccattgctcatggacatagactcattccttatgattgggagattctggcaaaatcatctctctcaccctctcaatttttacaatttaagacttggtgaattgatggggcacaagaacaggtccgaagaaatagggctgccaatcctccagttaacatagatgcagatcaactattaggaacaggtcaaaattggagcactattagtcaacaagcattaatgcaaaatgaggccattgagcaagttagagctatctgccttagagcctgggaaaaaatccaagacccaggaagcgcctgctccacatttaatacagtaagacaaggttcaaaagagccctaccctgattttgtggcaaggctccaagatgttgctcaaaagtcaattgccagtgaaaaagcccgtaaggtcatagtggagttgatggcatacgaaaacgccaatcctgagtgtcaatcagccattaagccattaaaaggaaaggttcccgcaggatcagatgtaatctcagagtatgtaaaagcccgtgatggaattggaggagctacgcataaagctatgcttatggcccaagcaataacaggagttgttttaggaggacaagttagaacatttggaggaaaatgttataattgtggtcaaattggtcatttaaaaaagaattgcccagtcttaaataaacagaatataactattcaagctactacaacaacaggtagagagccacctgacttatgtccaagatgtaaaaaaggaaaacattgggctagtcaatgtcattctaaatttgataaaaatgggcaatcattgtcgggaaactaccaaaagggctagtcaatgtcgttccaaatttgataaaaatgggcaaccattgtcgggaaactagcaaaggggccagcctcaggccctgcaacaaactggggcattcccaattcagccctttgttcctcagggttttcagggacaacaacccccactgtcccaagtacctcagggaataagccagttaccacagtacaacaattgtcccccgccacaagtggcagtgcagcagtagatttatgtactatacaagcagtctctctgcttccaggggagcccccacaaaaaatccccacaggagtatatggcccgctgcctgaggagactgtaggactaatcttgggaagatcacgtctaaatctaaaaggagttcaaattcatactggtgtggttgattcagactataaaggtgaaattcaattggttattagctcttcaattccttggagtgccagtccaggagacaggattgctcgattattactcctgccatatattaaggttggaaatagtgaaataaaaagaacaggagggtttggaagcactgatccgacaggaaaggctgcatattgggcaagtcaggtctcagagaacagacctgtgtgtaaggccgttattcaaggaaaacagcttgaaggattggtagacactggagcagatgtctctatcattgctttaaatcagtggccaaaaaattggcctaaacaaaagactgttacaggacttgtcggcatagtcacagcctcagaagtgtatcagagtactgagattttacattgcttagggccacataatcaagaaagtactgttcagccaatgatcacttcaattcctcttaatctgtggggtcgagatttgttacaacaatggggtgcggaaatcaccatgaccgctacattatatagccccatgagtcaaaaaatcatgaccaagatgggatatataccaggaaagggactaggaaaaaatgaagatggcattaaagttccaattgaggctaaaataaatcacggaagagaaggaacagggtatcctttttaggggtgaccactgtagagcctcctaaacccataccgttaacttggaaaacagaaaaactggtgtgggtaaatcagtggccgctaccaaaacaaaaactggaggctttacatttattagcaaatgaacagttagaaaagggacatattgagccttcattctcgccttggaattctcctgtgtttgtaattcagaagaaatccagcaaatggcgtatgttaactgacttaagggctgtaaatgccgtaattcaacccatggggcctctccaacctgggttgccctctccagccatgatcccaaaagattggcctttaattataattgatctaaaggactgcttttttaccatccctctggcagagcaggattgtgaaaaatttgcctttactataccagccataaataataaagaaccagccaccaggtttcagtggaaagtgttacctcagggaatgcttaatagtccaactatttgtcagacttttgtaggtcgagctcttcaaccagttagagacaagttttcagactgttatattattcattattttgatgatattttatgtgctgcagaaacgaaagataaattaattgactgttatacatttctgcaagcagaggttgccaatgcaggactggcaatagcatctgataagatccaaacctctactccttttcattatttagggatgcagatagaaaatagaaaaattaagccacaaaaaatagaaataagaaaagacacattaaaaacactaaatgattttcaaaaattgctgggagatattaattggattcggccaactctaggcattcctacttatgccatgtcaaatttgttctctatcttaagaggagactcagacttaaatagtaaaagaatgttaaccccagaggcaacaaaagaaattaaattagtggaagaaaaaattcagtcagcgcaaataaatagaatagatcccttagccccactccaacttttgatttttgccactgcacattctccaacaggcatcattattcaaaatactgatcttgtggagtggtcattccttcctcacagtacagttaagacttttacattgtacttggatcaaatagctactttaattggtccgacaagattacgaataataaaattatgtggaaatgacccagacaaaatagttgtccctttaaccaaggaacaagttagacaagcctttatcaattctggtgcatggcagattggtcttgctaattttgtgggaattattgataatcattacccaaaaacaaaaatcttccagttcttaaaattgactacttggattctacctaaaattaccagatgtgaacctttagaaaatgctctaacagtatttactgatggttccagcaatggaaaagtggcttacacagggccaaaagaacgagtaatcaaaactccatatcaatcggctcaaagagcagagttggttgcagtcattacagtgttacaagattttgatcaacctatcaatattatatcagattctgcatatgtagtacaggctacaagggatgttgagacagctctaattaaatatagcatggacgatcagttaaaccagctattcaatttattacaacaaactgtaagaaaaagaaacttcccattttatattactcatattcgagcacacactaatttaccagggcctttgactaaagcaaatgaacaagctgacttactggtatcatctgcattcataaaagcacaagaacttcatgctttgactcatgtaaatgcagcaggattaaaaaacaaatttgatgtcacatagaaacaggcaaaagatattgtacaacattgcacccagtgtcaagtcttagacctgcccactcaagaggcaggagttaacccagaggtctgtgtcctaatgcattatggcaaatggatgtcacgcatgtaccttcatttggaagattatcatatgtcatgtaacagttgatacttattcacatttcatgtgggcaacttgccaaacaggagaaagtacttcccatgttaaaaaacatttattgtcttgttttgctgtaatgggagttccagaaaaaatcaaaactgacaatggaccaggatattgtagtaaagctttccaaaaattcttaagtcagtggaaaatttcacatacaacaggaattccttataattcccaaggacaggccatagttgaaagaactaatagaacactcaaaactcaattagttaaacaaaaagaagggggagacagtaaggagtgtaccactcctcagatgcaacttaatctagcactctatactttaaattttttaaacatttatagaaatcagactactacttctgcagaacatcttactggtaaaaagaacagcccacatgaaggaaaactaatttagtggaaagataattaaaataagacatgggaaatagggaagctgataacgtgggggagaggttttgcttgtgtttcaccaggagaaaatcagcttcctgtttggatacccactagacatttgaagttctacaatgaacccatcagagatgcaaagaaaagcgcctccacggagatggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattatttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcgaaactattatagattgggcacctcgaggtcaattctaccacaattgctcaggacaaactcagtcgtgtccaagtgcacaagtgagtccagctgttgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcataaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcattttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcattgtgtgcctgttttgtctgttgttagtctgcaggtgtacccaacagctccgaagagacagtgacatcgagaacgggccatgatgacgatggcggttttgtcgaaaagaaaagggggaaatgtggggaaaagcaagagagatgagattgttactgtgtctgtatagaaagaagtagacataggagactccattttgttctgtactaagaaaaattcttctgccttgagatgctgttaatctatgaccttacccccaaccccgtgctctctgaaacatgtgctgtgtcaaactcagggttaaatggattaagggcggtgcaagatgtgctttgttaaacagatgcttgaaggcagcatgctcattaagagtcatcaccactccctaatctcaagtacccagggacacaaacactgcgaaagaccgcagggacctctgcctaggaaagctaggtattgtccaaggtttctccccatgtgatagtctgaaatatggcctcgtgggaagggaaagacctgaccatcccccagaccaacacccgtaaagggtctgtgctgaggaggattagtataagaggaaagcatgcctcttgcagttgagagaagaggaagacatctgtctcctgcccatccctgggcaatggaatgtctcagtataaaacccgattgaacattccatctactgagatagggaaaaactgccttagggctggaggtgggacatgtgggcagcaatactgctttgtaaagcattgagatgtttatgtgtatgcatatctaaaagcacagcacttgatcctttaccttgtctatgatgcaaagacctttgttcacgtgtttgtctgctcaccctctccccactattgtcttgtgaccctgacacatccccctctcggagaaacacccacgaatgatcaataaatactaagggaactcagaggctggcgggatcctccatatgctgaacgttggttccccgggcccccttatttctttctctatactttgtctctgtgtctttttcttttccaagtctctcgttccaccttatgagaaacacccacaggtgtggaggggcaacccaccccttcatgagacaatatttaaaggtttggggaaatcctgtaaggcagtaatcacagcaattaactccgccttttgagcagaagtataagaggtagaaataagtttgtctgtaggacttacatagccagcattgccattactggagccatcagtgaacactgtaacggcctcaggaatgggttgatttttggttaatcgaggaaccacctaagaagtcatttttataaaatcaaataatttgttttttggataatgattgtcaataacgccaataaaatcagccaagtgaatttgccacagtacagaatgttgaaaggcagcttgaactttgagccaatttaaaggaactacaattatatttggatcaaatccagaaatttgaagtattctgcaccaagcctgtctaattaatatggctatttggtctagataaacagacaaagtttttgagacagaataaggaagaaaacatcactccactaaatcattatgttgaactattagtccagtaggggagtgtaatgaagcaaaaactagaagctgaaaaggctgaaatggctgtactctagataactgggc

SEQ ID NO: 43 - DNA, 9343 bp, Human endogenous retrovirus
  misc_feature (1)..(9343): Where n is G or A or T or C
tgtggggaaaagcaacagaggtcagattgttactgtgtctgtatagaaagaagtagacatnaggagactccattttgttctgtactaagaaaaattattctgccttgagatgctgttaatcntatgaccttacccccaaccccgtgctctctgaaacatgtgctgtgtcaaactcagggttanaatggattaagggcggtgcaagatgtgctttgttaaacagatgcttgaaggcagcatgctncattaagagtcatcaccactccctaatctcaagtacccagggacacaaaaactgcggaagngctgcaggggcctctgcctaggaaagccaggtattgtccaaggtttctccccatgtgagangtctgaaatatggcctcgtgggaagggaaagacctgaccgtcccccagcccgacacccatnaaagggtctgtgctgaggaggattagtataagaggaaagcatgcctcttgcagttgagacnaagaggaaggcatctgtttcccacccatccctgggcaatggaatgtctcggtataaaaccncgattgtacgttccacctactgagatagggagaaaccaccttagggctggaggtgggacantgcaggcagcaatactgctttgtaaagcattgagatgtttatgtgtatgcatatctaaaangcacagcatttaatcctttaccttgtctatgatgcaaagacctttgttcacgtgtttgtcntgctcaccctctccccactattgtcttgtgaccctgacacatctccctctcagagaaacancccacgaatgatcaataaatactaaggggactcagaggctggtgggatcctccatatgctngaacgttggttccccgggcccccttatttctttctctatactttgtctctgtgtctttttncttttccaagtctctcattgcaccttacgagaaacacccacaggtgtggaggggcaacccnaccccttcatctggtgcccaacgtggaggcttttctctggggtgaaggtacactcgagcgntggtcattgaggacaagtcgacaagagatcccgagtacatctacagtcagccttacggtanagcttgtgcactcggaagaagctagggtgacaatggggcaaactaaaactaaaagtaaatnatgcctcttatcttagcttcattaaaattcttttaaaaagagggggagttagagtatccanccaaaaatctaatcaagctatttcaaacaacagaacaattttgcccatggtttccagaacnaaggaaatttagatctagaagattggaaaagaattggtaaggaactaaaacaagcaggtanggaagggtaatatcattccacttacagtatggaatgattggcccattattaaagcagcttntagaaccatttcaaacagaagatagcgtttcagtttctgatgcccctggaagctgtataantagattgtaatgaaaagacaaggaaaaaatcccagaaggaaacggaaactttacattgcgnaatatgtagcagagccgttaatggctcagtcaacgcaaaatgttgactataatcaattacnaggaggtgatatatcctgaaacattaaaattagaaggaaaaggtccagaattagtggggcncattagagtctaaaccacgagggccaagtcctctttcagcaggtcaggtgaccgtaacatntacaacctcaagcgcaggttagagaaaataagacccaactgccagtagcttatcaatactnggccaccggccgaacttcagtatcggccacccccagaaagtcagtatggatatctaggaantgccaccagcaccacaggacagggagccataccctcagccgcccactaggagacaatgctnatggcaccacctagtaggcagggtagtgaattacatgaaattattgagaagtcaagaaagngaaggagatactgaggcgtggcaattcccagtaacgttagaaccgatgccacctggagaanggagcccaagagggagagcctctcacagttgaggccagataaaggtctttttagataaaanatgctaaaagatatgaaagagggagtaaaacagtatggacccaactccccttatatgaggnacattattagattccattgctcatggacatagactcattccttatgattgggagattctgngcaaaatcatctctctcaccctctcaatttttacaatttaagacttggtgaattgatgggngcacaagaacaggtccgaagaaatagggctgccaatcctccagttaacatagatgcagatncaactattaggaacaggtcaaaattggagcactattagtcaacaagcattaatgcaaaatngaggccattgagcaagttagagctatctgccttagagcctgggaaaaaatccaagacccanggaagcgcctgctccacatttaatacagtaagacaaggttcaaaagagccctaccctgatntttgtggcaaggctccaagatgttgctcaaaagtcaattgccaatgaaaaagcccgtaagngtcatagtggagttgatggcatacgaaaacgccaatcctgagtgtcaatcagccattaagnccattaaaaggaaaggttcccgcaggatcagatgtaatctcagagtatgtaaaagcccgtngatggaattggaggagctacgcataaagctatgcttatggcccaagcaataacaggagttngttttaggaggacaagttagaacatttggaggaaaatgttataattgtggtcaaaatggtncatttaaaaaagaattgcccagtcttaaataaacagaatataactattcaagctactacanacaacaggtagagagccacctgacttatgtccaagatgtaaaaaaggaaaacattgggctnagtcaatgtcattctaaatttgataaaaatgggcaatcattgtcgggaaactaccaaaagnggctagtcaatgtcgttccaaatttgataaaaatgggcaaccattgtcgggaaactagcanaaggggccagcctcaggccctgcaacaaactggggcattcccaattcagccctttgttccntcagggttttcagggacaacaacccccactgtcccaagtacctcagggaataagccagttnaccacagtacaacaattgtcccccgccacaagtggcagtgcagcagtagatttatgtactnatacaagcagtctctctgcttccaggggagcccccacaaaaaatccccacaggagtatatnggcccgctgcctgaggagactgtaggactaatcttgggaagatcacgtctaaatctaaaanggagttcaaattcatactggtgtggttgattcagactataaaggtgaaattcaattggttnattagctcttcaattccttggagtgccagtccaggagacaggattgctcgattattactcnctgccatatattaaggttggaaatagtgaaataaaaagaacaggagggtttggaagcactngatccgacaggaaaggctgcatattgggcaagtcaggtctcagagaacagacctgtgtgtnaaggccgttattcaaggaaaacagcttgaaggattggtagacactggagcagatgtctctnatcattgctttaaatcagtggccaaaaaattggcctaaacaaaaggctgttacaggacttngtcggcgtaggcacagcctcagaagtgtatcaaagtactgagattttacattgcttagggnccacataatcaagaaagtactgttcagccaatgatcacttcaattcctcttaatctgtggnggtcgagatttgttacaacaatggggtgcggaaatcaccatgaccgctacattatatagcncccatgagtcaaaaaattatgaccaagatgggatatataccaggaaagggactaggaaaanaatgaagatggcattaaagttccaattgaggctaaaataaatcacggaagagaaggaacangggtatcctttttaggggtgaccactgtagagcctcctaaacccataccgttaacttgganaaacagaaaaactggtgtgggtaaatcagtggccactaccaaaacaaaaactggaggcttntacatttattagcaaatgaacagttagaaaagggacatattgagccttcattctcgccttnggaattctcctgtgtttgtaattcagaagaaatccagcaaatggcgtatgttaactgactntaagggctgtaaatgccgtaattcaacccatggggcctctccaacctgggttgccctctcncagccatgatcccaaaagattggcctttaattataattgatctaaaggactgcttttttanccatccctctggcagagcaggattgtgaaaaatttgcctttactataccagccataaatanataaagaaccagccaccaggtttcagtggaaagtgttacctcagggaatgcttaatagtcncaactctttgtcagacttttgtaggtcgagctcttcaaccagttagagacaagttttcagnactgttatattattcattattttgatgatattttatgtgctgcagaaacgaaagataaatntaattgactgttatacatttctgcaagcagaggttgccaatgcaggactggcaatagcatnctgataagatccaaacctctactccttttcattatttagggatgcagatagaaaatagaanaaattaagccacaaaaaatagaaataagaaaagacacattaaaaacactaaatgattttcnaaaaattgctgggagatattaattggattcggccaactctaggcattcctacttatgccantgtcaaatttgttctctatcttaagaggagactcagacttaaatagtaaaagaatgttaanccccagaggcaacaaaagaaattaaattagtggaagaaaaaattcagtcagcgcaaataanatagaatagatcccttagccccactccaacttttgatttttgccactgcccattctccaancaggcatcattattcaaaatactgatcttgtggagtggtcattccttcctcacagtacagnttaagacttttacattgtacttggatcaaatagctactttaattggtccgacaagattacngaataataaaattatgtggaaatgacccagacaaaatagttgtccctttaaccaaggaacnaagttagacaagcctttatcaattctggtgcatggcagattggtcttgctaattttgtggngaattattgataatcattacccaaaaacaaaaatcttccagttcttaaaattgactacttnggattctacctaaaattaccagacgtgaacctttagaaaatgctctaacagtatttactgnatggttccagcaatggaaaagtggcttacacagggccaaaagaacgagtaatcaaaactcncatatcaatcggctcaaagagcagagttggttgcagtcattacagtgttacaagattttgnatcaacctatcaatattatatcggattctgcatatgtagtacaggctacaagggatgttgnagacagctctaattaaatatagcatggacgatcagttaaaccagctattcaatttattacnaacaaactgtaagaaaaagaaacttcccattttatattactcatattcgagcacacactanatttaccagggcctttgactaaagcaaatgaacaagctgacttactggtatcatctgcatntcataaaagcacaagaacttcatgctttgactcatgtaaatgcagcaggattaaaaaacanaatttgatgtcacatggaaacaggcaaaagatattgtacaacattgcacccagtgtcaagntcttagacctgcccactcaagaggcaggagttaacccagaggtctgtgtcctaatgcattnatggcaaatggatgtcacacatgtaccttcatttgggaagattatcatatgttcatgtaancagttgatacttattcacatttcatgtgtgcaacttgccaaacaggagaaagtacttcccnatgttaaaaaacatttattgtcttgttttgctgtaatgggagttccagaaaaaatcaaaanctgacaatggaccaggatattgtagtaaagctttccaaaaattcttaagtcagtggaaaantttcacatacaacaggaattccttataattcccaaggacaggccatagttgaaagaactanatagaacactcaaaactcaattagttaaacaaaaagaagggggagacagtaaggagtgtanccactcctcagatgcaacttaatctagcactctatactttaaattttttaaacatttatangaaatcagactactacttctgcagaacatcttactggtaaaaagaacagcccacatgaagngaaaactaatttagcggaaagataattaaaataagacatgggaaatagggaagctgataancgtgggggagaggttttgcttgtgtttcaccaggagaaaatcagcttcctgtttggatacccactagacatttgaagttctacaatgaacccatcagagatgcaaagaaaagcgcctccacggagatggtaacaccagtcacatggatggataatcctatagaagtatatgttaatgatagtgtatgggtacctggccccacagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcctaacagtagattcacttatcacatggtaagcgggatgtcactcaggccacgggtaaattgtttacaagacttttcttatcaaagatcattaaaatttagacctaaagggaaaacttgccccaaggaaattcctaaaggatcaaagaatacagaagttttagtttgggaagaatgtgtggccaatagtgtggtgatattacaaaacaatgaattcggaactattatagattgggcacctcgaggtcaattctaccacaattgctcaggacaaactcagtcgtgtccaagtgcacaagtgagtccagctgtcgatagcgacttaacagaaagtctagacaaacataagcataaaaaattacagtctttctacctttgggaatgggaagaaaaaggaatctctaccccaagaccaaaaataataagtcctgtttctggtcctgaacatccagaattgtggaggcttactgtggcctcacaccacattagaatttggtctggaaatcaaactttagaaacaagatatcgtaagccattttatactatcgacctaaattccattctaacggttcctttacaaagttgcgtaaagcccccttatatgctagttgtaggaaatatagttattaaaccagcctcccaaactataacctgtgaaaattgtagattgtttacttgcattgattcaacttttaattggcagcaccgtattctgctggtgagagcaagagaaggcatgtggatccctgtgtccacggaccgaccgtgggaggcctcgccatccatccatattttgactgaaatattaaaaggcgttttaaatagatccaaaagattcatttttactttaattgcagtgattatgggattaattgcagtcacagctacggctgctgtggcaggagttgcattgcactcttctgttcagtcagtaaactttgttaattattggcaaaagaattctacaagattgtggaattcacaatctagtattgatcaaaaattggcaagtcaaattaatgatcttagacaaactgtcatttggatgggagacaggctcatgaccttagaacatcatttccagttacagtgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttggattaagaccatcagaagtactatgattataaatctcatattaatcgttgtgtgcctgttttgtctgttgttagtctgcaggtgtacccaacagctccgaagagacagtgacatcgagaacgggccatgatgacgatggcggttttgtcgaaaagaaaagggggaaatgtggggaaaagcaagagagatgagattgttactgtgtctgtatagaaagaagtagacataggagactccattttgttctgtactaagaaaaattcttctgccttgagatgctgttaatctatgaccttaccccnaaccccgtgctctctgaaacatgtgctgtgtcaaactctgggttaaatggattaagggtggtgcaagatgtgctttgttaaacagatgcttgaaggcagcatgctcattaagagtcatcaccactccctaatctcaagtacccagggacacaaacactgcgaaaggccgcagggacctctgcctaggaaagccaggtattgtccaaggtttctccccatgtgagagtctgaaatatggcctcgtgggaagggaaagacctgaccatcccccagaccgacacccgtaaagggtctgtgctgaggaggattagtataagaggaaagcatgcctcttgcagttgagagaagaggaagacatctgtttcctgcccatccctgggcaatggaatgtctcagtataaaacccgattgaacattccatctactgagatagggaaaaactgccttagggctggaggtgggacatgtgggcagcaatactgcttcgtaaagcattgagatgtttatgtgtatgcatatctaaaagcacagcacttgatcctttaccttgtctatgatgcaaacacctttgttcacgtgtttgtctgctgaccctctccccactattgtcttgtgaccctgacacatccccctctcggagaaacacccacgaatgatcaataaatactaagggaactcagaggctggcgggatcctccatatgctgaacgctggttccccngggcccccttatttctttctctatactttgtctctgtgtctttttcttttccaagtctctncattccaccttatgagaaacacccacaggtgtggaggggcaacccaccccttca

SEQ ID NO: 44 - DNA, 27 bp, Artificial Sequence
  Description of Artificial Sequence oligonucleotide primer
ccccaaacctttaaatattgtctcatg

SEQ ID NO: 45 - DNA, 20 bp, Artificial Sequence
  Description of Artificial Sequence oligonucleotide primer
ctttgataagaaaagtcttg

SEQ ID NO: 46 - DNA, 15 bp, Artificial Sequence
  Description of Artificial Sequence oligonucleotide primer
tgacctcgaggtgcc